SEQUENCE LISTING (18 sequences)

SEQ ID NO: 1 - DNA, 1788 bp, Homo sapiens
atggccgagagtggtgaaagcggcggtcctccgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaacgaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccagtggcacttctgggcagagtactactgcgccaaatttggtgcctggagtaggagctagtatgttcaacacaccaggaatgcagagcttgttgcaacaaataactgaaaacccacaacttatgcaaaacatgttgtctgccccctacatgagaagcatgatgcagtcactaagccagaatcctgaccttgctgcacagatgatgctgaataatcccctatttgctggaaatcctcagcttcaagaacaaatgagacaacagctcccaactttcctccaacaaatgcagaatcctgatacactatcagcaatgtcaaaccctagagcaatgcaggccttgttacagattcagcagggtttacagacattagcaacggaagccccgggcctcatcccagggtttactcctggcttgggggcattaggaagcactggaggctcttcgggaactaatggatctaacgccacacctagtgaaaacacaagtcccacagcaggaaccactgaacctggacatcagcagtttattcagcagatgctgcaggctcttgctggagtaaatcctcagctacagaatccagaagtcagatttcagcaacaactggaacaactcagtgcaatgggatttttgaaccgtgaagcaaacttgcaagctctaatagcaacaggaggtgatatcaatgcagctattgaaaggttactgggctcccagcatcatagcagcatttctgtatcttga

SEQ ID NO: 2 - protein, 595 aa, Homo sapiens
MAESGESGGPPGSQDSAAGAEGAGAPAAAASAEPKIMKVTVKTPKEKEEFAVPENSSVQQFKEEISKRFKSHTDQLVLIFAGKILKDQDTLSQHGIHDGLTVHLVIKTQNRPQDHSAQQTNTAGTNVTTSSTPNSNSTSGSATSNPFGLGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLSAAQEQFGGNPFASLVSNTSSGEGSQPSRTENRDPLPNPWAPQTSQSSSASSGTASTVGGTTGSTASGTSGQSTTAPNLVPGVGASMFNTPGMQSLLQQITENPQLMQNMLSAPYMRSMMQSLSQNPDLAAQMMLNNPLFAGNPQLQEQMRQQLPTFLQQMQNPDTLSAMSNPRAMQALLQIQQGLQTLATEAPGLIPGFTPGLGALGSTGGSSGTNGSNATPSENTSPTAGTTEPGHQQFIQQMLQALAGVNPQLQNPEVRFQQQLEQLSAMGFLNREANLQALIATGGDINAAIERLLGSQHHSSISVS

SEQ ID NO: 3 - DNA, 2974 bp, Homo sapiens
ggaggaagcggtggctgctgcggatgtcggtgtgagcgagcggcgcctgaacacacggcggctgccgagcgcctgacccgggcctgcgccagagcctgcaccgagctccggggccccacacccgctacggtggccctgcgcccgttgctactgaggcggcgtgctctgcattcttcgctgtccaggcctgccggctctggtgtctgctggctcctccttgctcgcctgctccctcctgcttgcctgagtcaccgccgccgccgccgccacagccatggccgagagtggtgaaagcggcggtcctccgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaagcaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccagtggcacttctgggcagagtactactgcgccaaatttggtgcctggagtaggagctagtatgttcaacacaccaggaatgcagagcttgttgcaacaaataactgaaaacccacaactgatgcaaaacatgttgtctgccccctacatgagaagcatgatgcagtcactaagccagaatcctgaccttgctgcacagatgatgctgaataatcccctatttgctggaaatcctcagcttcaagaacaaatgagacaacagctcccaactttcctccaacaaatgcagaatcctgatacactatcagcaatgtcaaaccctagagcaatgcaggccttgttacagattcagcagggtttacagacattagcaacggaagccccgggcctcatcccagggtttactcctggcttgggggcattaggaagcactggaggctcttcgggaactaatggatctaacgccacacctagtgaaaacacaagtcccacagcaggaaccactgaacctggacatcagcagtttattcagcagatgctgcaggctcttgctggagtaaatcctcagctacagaatccagaagtcagatttcagcaacaactggaacaactcagtgcaatgggatttttgaaccgtgaagcaaacttgcaagctctaatagcaacaggaggtgatatcaatgcagctattgaaaggttactgggctcccagccatcatagcagcatttctgtatcttgaaaaaatgtaatttatttttgataacggctcttaaactttaaaatacctgctttatttcattttgactcttggaattctgtgctgttataaacaaacccaatatgatgcattttaaggtggagtacagtaagatgtgtgggtttttctgtatttttcttttctggaacagtgggaattaaggctactgcatgcatcacttctgcatttattgtaattttttaaaaacatcaccttttatagttgggtgaccagattttgtcctgcatctgtccagtttatttgctttttaaacattagcctatggtagtaatttatgtagaataaaagcattaaaaagaagcaaatcatttgcactctataatttgtggtacagtattgcttattgtgactttggcatgcatttttgcaaacaatgctgtaagatttatactactgataattttgttttatttgtatacaatatagagtatgcacatttgggactgcatttctggaaacatactgcaataggctctctgagcaaaacacctgtaactaaaaaagtgaagataagaaaatactcttaaagctgagtatttcctaattgtatagaatcttacagcatctttgacaaacatctcccagcaaaagtgccggttagtcaggtttgttgaaaatacagtagaaaagctgattctggttatctctttaaggacaattaattgtacagacacataatgtaacattgtctcaacattcattcacagattgactgtaaattaccttaatctttgtgcagactgaaggaacactgtagtataccccaaagtgcatttgcctaggacttctcagcttctcccataggtagtttaacaggcattaaaatttgtaattgaaatgttgctttcactcaaaaaaaaaaaaaaaaaaaa

SEQ ID NO: 4 - protein, 589 aa, Homo sapiens
MAESGESGGPPGSQDSAAGAEGAGAPAAAASAEPKIMKVTVKTPKEKEEFAVPENSSVQQFKEEISKRFKSHTDQLVLIFAGKILKDQDTLSQHGIHDGLTVHLVIKTQNRPQDHSAQQTNTAGSNVTTSSTPNSNSTSGSATSNPFGLGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLSAAQEQFGGNPFASLVSNTSSGEGSQPSRTENRDPLPNPWAPQTSQSSSASSGTASTVGGTTGSTASGTSGQSTTAPNLVPGVGASMFNTPGMQSLLQQITENPQLMQNMLSAPYMRSMMQSLSQNPDLAAQMMLNNPLFAGNPQLQEQMRQQLPTFLQQMQNPDTLSAMSNPRAMQALLQIQQGLQTLATEAPGLIPGFTPGLGALGSTGGSSGTNGSNATPSENTSPTAGTTEPGHQQFIQQMLQALAGVNPQLQNPEVRFQQQLEQLSAMGFLNREANLQALIATGGDINAAIERLLGSQPS

SEQ ID NO: 5 - DNA, 1770 bp, Homo sapiens
atggccgagagtggtgaaagcggcggtcctccgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaagcaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccagtggcacttctgggcagagtactactgcgccaaatttggtgcctggagtaggagctagtatgttcaacacaccaggaatgcagagcttgttgcaacaaataactgaaaacccacaacttatgcaaaacatgttgtctgccccctacatgagaagcatgatgcagtcactaagccagaatcctgaccttgctgcacagatgatgctgaataatcccctatttgctggaaatcctcagcttcaagaacaaatgagacaacagctcccaactttcctccaacaaatgcagaatcctgatacactatcagcaatgtcaaaccctagagcaatgcaggccttgttacagattcagcagggtttacagacattagcaacggaagccccgggcctcatcccagggtttactcctggcttgggggcattaggaagcactggaggctcttcgggaactaatggatctaacgccacacctagtgaaaacacaagtcccacagcaggaaccactgaacctggacatcagcagtttattcagcagatgctgcaggctcttgctggagtaaatcctcagctacagaatccagaagtcagatttcagcaacaactggaacaactcagtgcaatgggatttttgaaccgtgaagcaaacttgcaagctctaatagcaacaggaggtgatatcaatgcagctattgaaaggttactgggctcccagccatcatag

SEQ ID NO: 6 - protein, 589 aa, Homo sapiens; identical to SEQ ID NO: 4

SEQ ID NO: 7 - DNA, 2354 bp, Homo sapiens
ggaggaagcggtggctgctgcggatgtcggtgtgagcgagcggcgcctgaacacacggcggctgccgagcgcctgacccgggcctgcgccagagcctgcaccgagctccggggccccacacccgctacggtggccctgcgtccaggcctgccggctctggtgcctgagtcaccgccgccgtcctccgggctcccaggatatgcctccgcggagcccaaaaattcgccgtgcccgagaatataaatcacatactgaccaactaccttgagtcagcatggaaaaacaggcctcaggatcattatcatcaactcctaatagtaaggtggccttgggggacttgtgaactacagagtcagatgccatggaaaatccctttgttctatggccaatccacaaatgcgaataatccagatataatgaggagatgatgaggaaccaggtaatgctttaaggcgcatgtgcagtttggtggtaatccattcaaccttcccgtacagaaaccagagttcatcagcttccatgccagtggcacttctgggctagtatgttcaacacaccagactgatgcaaaacatgttgtgaatcctgaccttgctgcactagagcaatgcaggccttgtcccgggcctcatcccagggtttcgggaactaatggatctacactgaacctggacatcagctcctcagctacagaatccaggggatttttgaaccgtgaagtgcagctattgaaaggttacaaaatgtaatttatttttgattgactcttggaattctgtggtacagtaagatgtgtgggtctactgcatgcatcacttcttgggtgaccagattttgtcctggtagtaatttatgtagaaaaaaaaaaaaaaaacccgttgctactgaggcggcgtgctctgcattcttcgctgtgtctgctggctcctccttgctcgcctgctccctcctgctccgccgccacagccatggccgagagtggtgaaagcggcgggcgccgccggagccgaaggtgctggcgcccccgcggccgctcatgaaagtcaccgtgaagaccccgaaggaaaaggaggagctccgtccagcagtttaaggaagaaatctctaaacgtttttgtgttgatatttgctggaaaaattttgaaagatcaagattcatgatggacttactgttcaccttgtcattaaaacacacagctcagcaaacaaatacagctggaagcaatgttactacactctacatctggttctgctactagcaacccttttggtttcaggtctgagtagcttgggtttgaatactaccaacttctcagcgacaacttttgtctaaccctgaaatgatggtccagatagagcatgctctcaaatcctgacctgatgagacagttaatagcagttgatacagagaaatccagaaattagtcatatgttgacaaacgttggaacttgccaggaatccagcaatgatgcaaccgagctttgagcaacctagaaagcatcccagggggataacacagatattcaggaaccaatgctgagtgctgcacaagattgcttccttggtgagcaatacatcctctggtgaaggtagatagagatccactacccaatccatgggctccacagacttcgcggcactgccagcactgtgggtggcactactggtagtacagagtactactgcgccaaatttggtgcctggagtaggagcgaatgcagagcttgttgcaacaaataactgaaaacccacactgccccctacatgagaagcatgatgcagtcactaagccaagatgcagaatcctgatacactatcagcaatgtcaaaccctacagattcagcagggtttacagacattagcaacggaagcttactcctggcttgggggcattaggaagcactggaggctcacgccacacctagtgaaaacacaagtcccacagcaggaacagtttattcagcagatgctgcaggctcttgctggagtaaaaagtcagatttcagcaacaactggaacaactcagtgcaatcaaacttgcaagctctaatagcaacaggaggtgatatcaatgggctcccagccatcatagcagcatttctgtatcttgaataacggctcttaaactttaaaatacctgctttatttcattctgttataaacaaacccaatatgatgcattttaaggtggattttctgtatttttcttttctggaacagtgggaattaagggcatttattgtaattttttaaaaacatcaccttttatagttgcatctgtccagtttatttgctttttaaacattagcctataaaagcattaaaaaaaaaaaaaaaaaaaaaaaaaaaaaa

SEQ ID NO: 8 - protein, 561 aa, Homo sapiens
MAESGESGGPPGSQDSAAGAEGAGAPAAAASAEPKIMKVTVKTPKEKEEFAVPENSSVQQFKEEISKRFKSHTDQLVLIFAGKILKDQDTLSQHGIHDGLTVHLVIKTQNRPQDHSAQQTNTAGSNVTTSSTPNSNSTSGSATSNPFGLGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLSAAQEQFGGNPFASLVSNTSSGEGSQPSRTENRDPLPNPWAPQTSQSSSASSGTASTVGGTTGSTASGTSGQSTTAPNLVPGVGASMFNTPGMQSLLQQITENPQLMQNMLSAPYMRSMMQSLSQNPDLAAQMQNPDTLSAMSNPRAMQALLQIQQGLQTLATEAPGLIPGFTPGLGALGSTGGSSGTNGSNATPSENTSPTAGTTEPGHQQFIQQMLQALAGVNPQLQNPEVRFQQQLEQLSAMGFLNREANLQALIATGGDINAAIERLLGSQPS

SEQ ID NO: 9 - DNA, 3952 bp, Homo sapiens
gggatgactcggcgacgccgccaggcgcgtagggacgtgggggaaggggcagggaggagggcgagcggcgcctgaacacacggcggctgcctgcaccgagctccggggccccacacccgcgcggcgtgctctgcattcttcgctgtccagccttgctcgcctgctccctcctgcttgccttggccgagagtggtgaaagcggcggtcctctacccggcgtgctccgcgcggcgccagcgaaagcggtggctgctgcggatgtcggtgtgacgagcgcctgacccgggcctgcgccagagctacggtggccctgcgcccgttgctactgaggcctgccggctctggtgtctgctggctcctgagtcaccgccgccgccgccgccacagccacgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaagcaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccacaaatttggtgcctggagtaggagctagtatgcaacaaataactgaaaacccacaactgagaagcatgatgcagtcactaagccagaatcatcccctatttgctggaaatcctcagcttctcctccaacaaatgcagaatcctgatacacaggccttgttacagattcagcagggtttactcccagggtttactcctggcttgggggcatatggatctaacgccacacctagtgaaaacagacatcagcagtttattcagcagatgctgcagaatccagaagtcagatttcagcaacaacaccgtgaagcaaacttgcaagctctaatagaaaggttactgggctcccagccatcatagctatttttgataacggctcttaaactttaaaaattctgtgctgttataaacaaacccaatatgtgtgggtttttctgtatttttcttttctatcacttctgcatttattgtaattttttaaattttgtcctgcatctgtccagtttatttgtatgtagaataaaagcattaaaaagaagcagtattgcttattgtgactttggcatgcattctgataattttgttttatttgtatacaataggaaacatactgcaataggctctctgagcagaaaatactcttaaagctgagtatttcctaaacatctcccagcaaaagtgccggttagtcattctggttatctctttaaggacaattaatacattcattcacagattgactgtaaattacgtagtataccccaaagtgcatttgcctaggcaggcattaaaatttgtaattgaaatgttggttatttttaatcgccataaaaaaatagaacacaggcaatacacaaatttaaaatgagttagttctattaagaaatagttaaatattgtgggtgggggggtggggcagcggaatctgtccaccaagattctgttaggatttctgtgcatagaaaaacaaagagccgttttaatgatgttggtggcacttctgggcagagtactactgcgctgttcaacacaccaggaatgcagagcttgttgcaaaacatgttgtctgccccctacatgactgaccttgctgcacagatgatgctgaataaagaacaaatgagacaacagctcccaactttatcagcaatgtcaaaccctagagcaatgcagacattagcaacggaagccccgggcctcataggaagcactggaggctcttcgggaactacaagtcccacagcaggaaccactgaacctgaggctcttgctggagtaaatcctcagctactggaacaactcagtgcaatgggatttttgacaacaggaggtgatatcaatgcagctattgagcatttctgtatcttgaaaaaatgtaattatacctgctttatttcattttgactcttggtgatgcattttaaggtggagtacagtaagaggaacagtgggaattaaggctactgcatgcaaacatcaccttttatagttgggttaccagctttttaaacattagcctatggtagtaattaatcatttgcactctataatttgtggtacatttgcaaacaatgctgtaagatttatactatagagtatgcacatttgggactgcatttctaaacacctgtaactaaaaaagtgaagataaattgtatagaatcttacagcatctttgacaaggtttgttgaaaatacagtagaaaagctgtgtacagacacataatgtaacattgtctcacttaatctttgtgcagactgaaggaacactacttctcagcttctcccataggtagtttaactttcactgaaaaagtgtcttgatgtttcactatcttttgggtttatctgttttctcatggtgagccaattgtttctgaagtgttttggtcttttcagagcctcagagaaagggggacggtggatggggccagcttaaataatactggcatagtgtagtaaagaagtatcattcaggggtagtacatttggctgttttatagcctttttcttccctcccccaaagaattctgtttgcctaactcccaaactgttggggtggtacattcctttaggaccaattaaaacataattgagggtcagtgatacatttggctgactctggttcagtattctcttaggtgattatattctctcatgtacagttacaggaaattaaaatgttaaagtaacctaaaatgaattcagaccaataaaatcaagggaaatacaagttgattgcattacttctgtatgttgcttgctattaaaaaggttaagaggccaggttacccaccagtccttgcactgttctgacactttccccaggaggaaaacaagtacaaaggttacggtggaggcataagtagaagagattgttaagaagggtattcatgtgtctttgctctttctgctttatgcctcagtttggtttaaaaacttctgtactggcaaatggtggtattcagtgtgggatagtgtcataactaatttgacaatttattaatcataaaataacaataaatctctagcttttacacttg

SEQ ID NO: 10 - protein, 589 aa, Homo sapiens; identical to SEQ ID NO: 4

SEQ ID NO: 11 - DNA, 3018 bp, Homo sapiens
atggctacagagagtgcactcatcaaagttcacgtgaaatcaccgtcgaacaagtatgactaccgatgtctctcacgtgagtagtttcaagtgcactttagtggcagcttgttcatactggttgagattgctgcggatgcatcagtttcagaactaaaagacaaggttcttgtgttcgttcaactctaacgacgcctacgtagtcaaagtcttgattttctgttccaagaacacaagcaaccaactgcgaacaaagaacaagtttgtataatttacaccggaaaaattctgaaggatgaaggttgacgcttgtttcttgttcaaacatattaaatgtggcctttttaagacttcctacttgaaactctcacacagcacaaaatcgctgatggtcacaccgtccacttggttattagaaatctttgagagtgtgtcgtgttttagcgactaccagtgtggcaggtgaaccaataatctttacaagcccgtccaacaccagcgccggctgctgcaacaccaacagcttcatctgcaccaagtgttcgggcaggttgtggtcgcggccgacgacgttgtggttgtcgaagtagacgtggttcatccaatccaacaccttcttcacaaccgaatcctaccaataatccatttgcagcaatgggaaggttaggttgtggaagaagtgttggcttaggatggttattaggtaaacgtcgttaccctggaatgggatcacctgctgatattttaaacaatccagatgctatgcgttcagttatggatccttaccctagtggacgactataaaatttgttaggtctacgatacgcaagtcaatacctaaatccaattacacaacaacttctaggaaatccagagtttatgagaacaattattcaatccttaggttaatgtgttgttgaagatcctttaggtctcaaatactcttgttaataagttaggaacccacaattccaagcattgattgagagaaatccagaagttggacacattctcaacgatttgggtgttaaggttcgtaactaactctctttaggtcttcaacctgtgtaagagttgctaccgaatgtaatgcgtcaaactatggagatgattcgtaatccaaatatgttccaagaaatgggcttacattacgcagtttgatacctctactaagcattaggtttatacaaggttctttacatgcggaatcatgatcaagctattaggaatcttcagggaattcccggaggagaagctgcttacgccttagtactagttcgataatccttagaagtcccttaagggcctcctcttcgacgacttgaacggttgtacaatgatgtacaggagcccttgctcaatagtgcaacaaattcgctcgaacttgccaacatgttactacatgtcctcgggaacgagttatcacgttgtttaagcgagagtggaaatccatttgcttctctaagaggtgatcagagcagtgagccacgtgtcgatcgttcacctttaggtaaacgaagagattctccactagtctcgtcactcggtgcacagctagcagctggacaagaaaataatgaagctctgccaaatccatgggcttcaaacgccaatcaagctcgacctgttcttttattacttcgagacggtttaggtacccgaagtttgcggttagttcgaaccaataatcaatcgaataatcgttctgctgactttaattcattgcttgattcacctggctggttattagttagcttattagcaagacgactgaaattaagtaacgaactaagtggaccgatcagctccctaatggagcagatgatgtccaatccaagtatgcaggccagtatgttcagctagtcgagggattacctcgtctactacaggttaggttcatacgtccggtcatacaagtcgccagaagtcatcaattcaattcgtcaaaatatgtcaaacaatcctggactcattgattccggtcttcagtagttaagttaagcagttttatacagtttgttaggacctgagtaactaaggattgttggacaaattccatcggctcgtgataacccacagatttccgaaggaattcgtagataacaacctgtttaaggtagccgagcactattgggtgtctaaaggcttccttaagcatctagttttccacaaatgctcaacatgatgtctgatccatctgtgatggaggcaatgagaaactcaaaaggtgtttacgagttgtactacagactaggtagacactacctccgttactctttgccacgggtcagtgaagcattccgtcaaattcaagagggattttcaactcttcgcagagaaggtgcccagtcacttcgtaaggcagtttaagttctccctaaaagttgagaagcgtctcttgcacctcaacttttgaaccttttccaagctggagcaatgggaggtggagcattcggttctcgtggagttgaaaacttggaaaaggttcgacctcgttaccctccacctcgtaagccaagagatgccaatgcatcttctgctggagcaaactctgcaaacggacttgccgatttattcaatctacggttacgtagaagacgacctcgtttgagacgtttgcctgaacggctaaataagttatcaatgaatatgggaggaggaagaccctcatcgactgctgcaccagtgaatccggagcaaagttacttataccctcctccttctgggagtagctgacgacgtggtcacttaggcctcgttacctatgcatcacaactcgaacaacttcaatcgatgggcttctcggatcgtgccagaaattggatacgtagtgttgagcttgttgaagttagctacccgaagagcctagcacggtctttagtggcagcgctgaccgcaacgttcggagatctcaacgcggctgtcgaacgtcttctcaaccaccgtcgcgactggcgttgcaagcctctagagttgcgccgacagcttgcagaagagttgtctccatagagaggtatc

SEQ ID NO: 12 - protein, 502 aa, Homo sapiens
MATESALIKVHVKSPSNKYDVEIAADASVSELKDKVLVFVPTANKEQVCIIYTGKILKDEETLTQHKIADGHTVHLVIRNQARPTPAPAAATPTASSAPSSNPTPSSQPNPTNNPFAAMGGMGSPADILNNPDAMRSVMDNPITQQLLGNPEFMRTIIQSNPQFQALIERNPEVGHILNDPNVMRQTMEMIRNPNMFQEMMRNHDQAIRNLQGIPGGEAALERLYNDVQEPLLNSATNSLSGNPFASLRGDQSSEPRVDRAGQENNEALPNPWASNANQATNNQSNNRSADFNSLLDSPGISSLMEQMMSNPSMQASMFSPEVINSIRQNMSNNPGLIDSIVGQIPSARDNPQISEGIRRSFPQMLNMMSDPSVMEAMRNPRVSEAFRQIQEGFSTLRREAPQLLNLFQAGAMGGGAFGSDANASSAGANSANGLADLFNSMNMGGGRPSSTAAPVNPEQTYASQLEQLQSMGFSDRARNVAALTATFGDLNAAVERLLNSP

SEQ ID NO: 13 - DNA, 3324 bp, Homo sapiens
cggaggaggcccagagaccggagcgcggagacctcagccagcggcctacgcccaggcctttctccaccggaggaccagggaaccgcagtcttcatcacagaggtaccgtgctccgcgctccccgcctgacccggcccagcccgctgcggcggtgcctccttccttcctccttccctcgcgctctctctttcgcccgcccgcgccttccctgcccgcctgcgtcaccgcggccgccatggctgagaatggcgagagcagcggccccccgcgcccctcccgcggccctgctgcggcccaaggctcggctgctgccccggctgagcctaaaatcatcaaagtcacggtgaagactcccaaagagaaagaggagttcgcggtgcccgagaacagctcggttcagcagtttaaggaagcgatttcgaaacgcttcaaatcccaaaccgatcagctagtgctgatttttgccggaaaaatcttaaaagatcaagataccttgatccagcatggcatccatgatgggctgactgttcaccttgtcatcaaaagccagaaccgacctcagggccagtccacgcagcctagcaatgccgcgggaactaacactacctcggcgtcgactcccaggagtaactccacacctatttccacaaatagcaacccgtttgggttggggagcctgggaggacttgcaggccttagcagcctgggcttgagctcgaccaacttctctgagctccagagccagatgcagcagcagcttatggccagccctgagatgatgatccaaataatggaaaatccctttgttcagagcatgctttcgaatcccgatctgatgaggcagctgattatggctaatccacagatgcagcaattgattcagagaaacccagaaatcagtcacctgctcaacaacccagacataatgaggcagacactcgaaattgccaggaatccagccatgatgcaagagatgatgagaaatcaagacctggctcttagcaatctagaaagcatcccaggtggctataatgctttacggcgcatgtacactgacattcaagagccgatgctgaatgccgcacaagagcagtttgggggtaatccatttgcctccgtggggagtagttcctcctctggggaaggtacgcagccttcccgcacagaaaatcgcgatccactacccaatccatgggcaccaccgccagctacccagagttctgcaactaccagcacgaccacaagcactggtagtgggtctggcaatagttccagcaatgctactgggaacaccgttgctgccgctaattatgtcgccagcatctttagtaccccaggcatgcagagcctgctgcaacagataactgaaaacccccagctgattcagaatatgctgtcggcgccctacatgagaagcatgatgcagtcgctgagccagaatccagatttggctgcacagatgatgctgaatagcccgctgtttactgcaaatcctcagctgcaggagcagatgcggccacagctcccagccttcctgcagcagatgcagaatccagacacactatcagccatgtcaaacccaagagcaatgcaggctttaatgcagatccagcaggggctacagacattagccactgaagcacctggcctgattccgagcttcactccaggtgtgggggtgggggtgctgggaaccgctataggccctgtaggcccagtcacccccataggccccataggccctatagtcccttttacccccataggccccattgggcccataggacccactggccctgcagccccccctggctccaccggctctggtggccccacggggcctactgtgtccagcgctgcacctagtgaaaccacgagtcctacatcagaatctggacccaaccagcagttcattcagcaaatggtgcaggccctggctggagcaaatgctccacagctgccgaatccagaagtcagatttcagcaacaactggaacagctcaacgcaatggggttcttaaaccgtgaagcaaacttgcaggccctaatagcaacaggaggcgacatcaatgcagccattgaaaggctgctgggctcccagccatcgtaatcacatttctgtacctggaaaaaaaatgtatcttatttttgataatggctcttaaatctttaaacacacacacaaaatcgttctttactttcattttgattcttttaaatctgtctagttgtaagtctaatatgatgcattttaagatggagtccctccctcctacttccctcactccctttctcctttgcttatttttcctaccttcccttcctcttgtctccccactccctccctctttgtttccttccttccttatttcctttagtttccttccttagccgttttgagtggtgggaatcaatgctgtttcactcaaaagtgttgcatgcaaacacttctctttattctgcatttattgtgatttttggaaacaggtatcaaccttcacagttgggtgaacaagtgttgtcctacagatgtccaatttatttgcatttttaaacattagcctatgatagtaatttaatgtagaatgaagatattaaaaacagaagcaaattatttgaagctctctaatttgtggtacgatattgcttattgtgactttggcatgtatttttgctagcaaaatgctgtaagatttataccattgatcttttttgctatatttgtatacagtacagtaagcacaattggcactgtacatctaaaaatattacagtagaatctgagtgtaatatgtgtaaccaaaatgagaaagaatacaagaaatgtttctggagctagttatgtctcacaattttgtagaatcttacagcatctttgataaacttctcagtgaaaatgttggctaggcaagttcagttaaaatatagtagaaatgtttatcctggtatctctaagtatacatttaattgtacagaaaatttacagtgtaacattgtgtcaacatttgcagattgactgtatatgaccttaatctttgtgcagcctgaaggatcagtgtagtaatgccaggaaagtgctttttacctaagacttccttctcagcttctcccataaagagaccctaatatgcattttgatttgtaattggaaatgtaactttcactgaaagtgtcatgtgatgtttgcattacttttaactgctatgtataaaggaaagtgtgtcttttgacttcatcagttatttctcttgtgcacagagaaaaatgcattaaaaatgactaaaaaaaataaaaaattaaaaaatgaaaaaaaaaaaaaaaaaa

SEQ ID NO: 14 - protein, 624 aa, Homo sapiens
MAENGESSGPPRPSRGPAAAQGSAAAPAEPKIIKVTVKTPKEKEEFAVPENSSVQQFKEAISKRFKSQTDQLVLIFAGKILKDQDTLIQHGIHDGLTVHLVIKSQNRPQGQSTQPSNAAGTNTTSASTPRSNSTPISTNSNPFGLGSLGGLAGLSSLGLSSTNFSELQSQMQQQLMASPEMMIQIMENPFVQSMLSNPDLMRQLIMANPQMQQLIQRNPEISHLLNNPDIMRQTLEIARNPAMMQEMMRNQDLALSNLESIPGGYNALRRMYTDIQEPMLNAAQEQFGGNPFASVGSSSSSGEGTQPSRTENRDPLPNPWAPPPATQSSATTSTTTSTGSGSGNSSSNATGNTVAAANYVASIFSTPGMQSLLQQITENPQLIQNMLSAPYMRSMMQSLSQNPDLAAQMMLNSPLFTANPQLQEQMRPQLPAFLQQMQNPDTLSAMSNPRAMQALMQIQQGLQTLATEAPGLIPSFTPGVGVGVLGTAIGPVGPVTPIGPIGPIVPFTPIGPIGPIGPTGPAAPPGSTGSGGPTGPTVSSAAPSETTSPTSESGPNQQFIQQMVQALAGANAPQLPNPEVRFQQQLEQLNAMGFLNREANLQALIATGGDINAAIERLLGSQPS

SEQ ID NO: 15 - DNA, 2347 bp, Homo sapiens
gggaggtttggagccctgcataaagagaaggacgggaccacagctgactgctgtgtccccacagatctgggcctcctgctgccaccatggccaaaggtggagaagccctgccacagggcagcccagcaccagtccaggatccccacctcatcaaggtgacagtgaagacgcccaaagacaaggaggatttctcagttacagacacatgcactatccagcagctgaaggaagagatatctcagcgctttaaggcccaccccgatcagcttgttctaatctttgctggcaaaatcctcaaggatcctgactcactggcacagtgtggagtgcgagatggcctcactgtccacctggtcatcaagaggcagcaccgtgccatgggcaatgagtgcccagctgcctctgtccctacccagggcccaagtcctggatcactccctcagccaagctccatttacccagcagatgggccccctgcctttagcttaggtctcctcacaggcctcagtaggctgggcttggcctatcgtggcttccctgaccagccaagctccctgatgcggcagcatgtgtctgtgcctgagtttgtgactcagctcattgatgaccccttcatcccgggtctgctgtccaacacaggcctagtacgccagctggttcttgacaacccccatatgcagcagctgatccagcacaaccctgagattgggcatattcttaacaacccggaaattatgcggcagacactggagtttttacgtaaccctgccatgatgcaggagatgatacgtagccaggaccgggtgctcagtaacttggagagcattcctggtggctacaatgtgctttgcactatgtacacagatattatggacccaatgcttaacgcagtccaggagcagtttggcggcaatccctttgccactgccactactgataatgccaccaccaccaccagccaaccttcaaggatggagaattgtgaccctctccccaacccctggacttccacacatggaggctcaggtagcaggcaaggaaggcaggatggggatcaggatgcacctgacattagaaataggtttccaaactttctgggtattataaggctctatgactatctccagcaattacacgagaacccccagtccctaggaacttatctacaggggactgcatctgccctcagccaaagccaggaaccaccaccatcagtaaacagagttcccccatcgtcaccctcatctcaggagcctgggtcaggccagcctctccccgaggagtcagtagcaatcaagggaaggtcctcctgcccagctttcctgagataccccacagagaacagtactggacaaggtggagaccaagatggtgcagggaaaagctctactggacatagcacaaacttgcctgatcttgtctcggggctgggagattctgccaacagggttccatttgctcccttatctttttcccccacggcagccattcctggaatccctgagcctccctggctgccatccccggcttatccaagatctctgaggccagatggcatgaatccagctccacagttacaggatgagatacaaccacagctgccactgctgatgcaccttcaggcagccatggcaaacccccgtgccctgcaagccctgcggcagattgagcagggtctacaggtcctagctactgaagcacctcgcctcctactctggttcatgccttgcctagcagggacgggtagtgtggcaggaggtatagagtctagagaagatccccttatgtctgaggatcctctcccaaatccacctcctgaggtgttcccagcactggactctgcagagctgggcttcctttcccctccctttctccatatgctgcaagatttagttagtacaaatccccagcagctgcagcctgaggctcactttcaggtgcagctggagcaactgcggtccatgggctttctgaatcgtgaagccaatcttcaggccctcattgctacggggggcgacgtggatgctgctgtggagaagctgagacagtcgtaggagccttattcattcaaaccatacgttttcctctgtgcctttttcccatatcctagttccctagctctcccatttttgaatacagctgcattataaaccaaatttactatgaagtcctttgctgtggaggcaatgttgttccagagtcaacgaggaagactaatggccaaaacatagtggaggtgctgtgtgtgagtcaaccacttgtaccactataccactggggggccccagtctaagctctgcttatgcctatcttgagatgcaattacacccaatttccaatgtgaaaa

SEQ ID NO: 16 - protein, 655 aa, Homo sapiens
MAKGGEALPQGSPAPVQDPHLIKVTVKTPKDKEDFSVTDTCTIQQLKEEISQRFKAHPDQLVLIFAGKILKDPDSLAQCGVRDGLTVHLVIKRQHRAMGNECPAASVPTQGPSPGSLPQPSSIYPADGPPAFSLGLLTGLSRLGLAYRGFPDQPSSLMRQHVSVPEFVTQLIDDPFIPGLLSNTGLVRQLVLDNPHMQQLIQHNPEIGHILNNPEIMRQTLEFLRNPAMMQEMIRSQDRVLSNLESIPGGYNVLCTMYTDIMDPMLNAVQEQFGGNPFATATTDNATTTTSQPSRMENCDPLPNPWTSTHGGSGSRQGRQDGDQDAPDIRNRFPNFLGIIRLYDYLQQLHENPQSLGTYLQGTASALSQSQEPPPSVNRVPPSSPSSQEPGSGQPLPEESVAIKGRSSCPAFLRYPTENSTGQGGDQDGAGKSSTGHSTNLPDLVSGLGDSANRVPFAPLSFSPTAAIPGIPEPPWLPSPAYPRSLRPDGMNPAPQLQDEIQPQLPLLMHLQAAMANPRALQALRQIEQGLQVLATEAPRLLLWFMPCLAGTGSVAGGIESREDPLMSEDPLPNPPPEVFPALDSAELGFLSPPFLHMLQDLVSTNPQQLQPEAHFQVQLEQLRSMGFLNREANLQALIATGGDVDAAVEKLRQS

SEQ ID NO: 17 - DNA, 3545 bp, Homo sapiens
gggcggccgggtggcggcggcggcatggcggagccgagcggggccgagacgaggccccccattcgggtcaccctcaagacccccaaggacaaggaggaaattgtgatctgcgatcgagcctcggtcaaggagttcaaagagtcctgatcttcgcaggcaaaaggacgggctcactgtccagctgccactgcttcttccccgcttcacccgccacccctgcagtggaagccggaggagcagagtgctactgcgtccatactctgggctctgccaacttcatcctgagatgctgtcacagatgatctgatgcgtcacatgatcctgagatcagccacatgctcggaatccagccatgatgcagagagcatccctggagggtaatgttcagtgctgcccgggatccgacagctcatcctcccatggagcccctcgccccccactcggggaccagccaggtgcactggggtcagggatgttcaaaacccccagctgatgcagaacttgcccagaaccccgacttaacccccaactgcaggagcaaacccagagtcactctccatggaaatctcccggaggtttaaggctcagcaggatcagctggatcctcaaggatggggacacactgaaccagcacggaatctctggtcatcaagacccctcagaaggctcaagatccagctctccacacctgaccctgcctcagcaccctccaccacgcctccagccctccacctctggcagtgcctcttcagatgctggctggtggggggccctctccgggggctggggagggatcccccctctggctttgggggcatcctggggctgggcagcctaggcggagctgcagcagcagatgcagcggcagctgatgtccaatcatggagaaccccctggtccaggatatgatgtctaacccttatggccaacccccagatgcagcagttgatggagcggaaccaataaccctgaactcatgaggcagacaatggagcttgctagagatgatgcggaaccaggaccgggccctgagcaacctttaatgccctccgccgcatgtacacggacatccaggagcccacagtttggcaacaatcccttctcttccctggccgggaacgcctctgcggactgagaatcgagagcccctccctaaccccctcccaggcccccgggtccggtggggagggcaccggaggacccgacagtctcgaacccctttgggatcaatgcggctagctagcccagaaatgcaagccctcctccagcagatctctgagtgtgatctcagcaccctacatgcgcagcatgatgcagacgtgctgctcagatgatggtgaatgtgccgctcttcgcgggggctccgcctgcagctcccagtcttcctgcagcagatgcagccttaccaatccccgagccatgcaggcattgctgcagatccagcagggactacagaccttgcagaccgaggcccctgggctggtacccagccttggctcctttgggatatcccggaccccgcccccacttcctcaccagccagcagcaactcatgcagcacagacgccagaagtgagattaatcgtgaggctaacctgcagagagactgctgggctcccatccctcgatgtcagcattcgttccgtcttctccctcatccccccttagctctgtctgagacctggtctccttgagcagtgcttcttgccccacaccacttgaggaaccagctctctggtttcttatgtttacttttggtacccaaccaatgctagaattttccttctcggcccctattctagcaccctcagcaggcagcaacgcagggtctacgcccgagcacgccagccacatcttctccaacaggggcttccagcgccgatgatccagcttttggctggaagtggaaactcacaggtgtcagcagcagctggagcagctcaactccatgggcttcatcggccctgattgccacaggaggggacatcaacgcagctatcgctctcctaatccctcggcccatgcctcctgcctctccccgttcttctgtcaatccttaccctctgcagcttgtcctccctttccaaacagcagggtgactttagaggcatgggctccaaattatggttttactgctacgtctctaacagactcttctctctacttaaacagttttcacagtttcattgattgactctacttgcaatctttaaactttcagtggctgtgcagagtcgaggtactggaacatagtcttccatctataccactagggttttgactctcttccttctttttctccctaccccccaacccctagcttgctctgaaggaggagcaggtgaaacaggtggtaatttggttcattcagcactttttggtgggaactgttggaaattccagggtaaggaggaagatgcctgttctccctgtctgaagagggagatgagacagctctctggacaggaattaacaaacgctggagcagcccagaggaaattcgtgtgaaagaggagggaatgagattattcggaggaagggaatgggggagacagcctgagtaaaaggcttggaagttggaattagcagtggggagcagaagcactcatagctcttttaggcagaagaatccaggcccgagctggcagaagagacttagagatgctaatggaatttaaactgaaaaaaggagcccaatgaagctaagcgccacgccccacaaggggtcatattggctttagttcctcaagcatatgtgcttatatgcacacacacacacatttccatggacccaggtcttgcttgtgtccccaggcaccagtagtttgagcccccctcaaaagacatgaagggggttggggtctgtgtgagtagtggggaggtgcatgtgtatccacatgtgtgcatacactcttaagttgggtgggaagtggattccttgttggtttctggttcagagtgctctcccaccaccagagacaagtgggtaaaaaggtcagtccattgcaggaatatatatccgggagagctaggtcccttggggctctggatgctgggtaacccagaggtaatgtgggtaccctttctgaagctgtcagggctgtgactagcacccttatcacccctcactgccttgtgggaatagtagagggtttttttcctccagagcccctggcctttcagttcttaactatttccctccaggccagaaagttttctttgaggaaggagaggagagggtggcaatgatgcctttgatctggaattggacatttctctgtcagagcacagaggaggctcatatcacctcttccctctcctacttggcccagctgcttggaggaccgaccccatggctgagaatatgacggcaagaggaacagagtttgctccaagtgggaaagggtcccaagcagtccagagaagatgtctgtgtggctttccctccctgcctcccccagctcccacactggcctttgtaaataaatggcgtggtctttgttgtgaaaaaaaaaaaaaaaaaaaaaaaaaaaaaaaaaaaaaaa

SEQ ID NO: 18 - protein, 601 aa, Homo sapiens
MAEPSGAETRPPIRVTVKTPKDKEEIVICDRASVKEFKEEISRRFKAQQDQLVLIFAGKILKDGDTLNQHGIKDGLTVHLVIKTPQKAQDPAAATASSPSTPDPASAPSTTPASPATPAQPSTSGSASSDAGSGSRRSSGGGPSPGAGEGSPSATASILSGFGGILGLGSLGLGSANFMELQQQMQRQLMSNPEMLSQIMENPLVQDMMSNPDLMRHMIMANPQMQQLMERNPEISHMLNNPELMRQTMELARNPAMMQEMMRNQDRALSNLESIPGGYNALRRMYTDIQEPMFSAAREQFGNNPFSSLAGNSDSSSSQPLRTENREPLPNPWSPSPPTSQAPGSGGEGTGGSGTSQVHPTVSNPFGINAASLGSGMFNSPEMQALLQQISENPQLMQNVISAPYMRSMMQTLAQNPDFAAQMMVNVPLFAGNPQLQEQLRLQLPVFLQQMQNPESLSILTNPRAMQALLQIQQGLQTLQTEAPGLVPSLGSFGISRTPAPSAGSNAGSTPEAPTSSPATPATSSPTGASSAQQQLMQQMIQLLAGSGNSQVQTPEVRFQQQLEQLNSMGFINREANLQALIATGGDINAAIERLLGSQLS

(19) United States
(12) Patent Application Publication
(10) Pub. No.: US 2011/0166206 A1
(43) Pub. Date: Jul. 7, 2011

(54) UBIQUILIN REGULATION OF PRESENILIN ENDOPROTEOLYSIS, AND SUPPRESSION OF POLYGLUTAMINE-INDUCED TOXICITY IN CELLS

(75) Inventors: Mervyn J. Monteiro, Columbia, MD (US); Hongmin Wang, Timonium, MD (US)

(73) Assignee: UNIVERSITY OF MARYLAND BIOTECHNOLOGY INSTITUTE, Baltimore, MD (US)

(21) Appl. No.: 11/911,779

(22) PCT Filed: Apr. 19, 2006
(86) PCT No.: PCT/US2006/014795

§ 371 Date: Jun. 10, 2009

Related U.S. Application Data
(60) Provisional application No. 60/673,642, filed on Apr. 21, 2005.
(60) Provisional application No. 60/706,839, filed on Aug. 9, 2005.

(51) Int. Cl. A61K 31/7088; C12Q 1/68; C12N 5/06

(52) U.S. Cl. 514/44 R; 435/375; 435/6.16

(57) ABSTRACT

Use of ubiquilin is described, including utilization for reducing fragmentation of presenilin 1 and 2 and to modulate γ-secretase components, Pen-2 and Nicastrin, as well as utilization for inducing increased levels of ubiquilin to reduce aggregation of polyglutamine expansion proteins known to cause cell toxicity and cell death in subjects suffering from neurodegenerative diseases, such as Huntington's and Alzheimer's diseases.

### BACKGROUND OF THE INVENTION

1. Field of the Invention

The present invention generally relates to ubiquilin. More specifically, the invention in one aspect relates to protein-protein interactions, in which ubiquilin functions as a presenilin interactor protein to reduce fragmentation of presenilin 1 and 2 and to modulate γ-secretase components, Pen-2 and Nicastrin. The invention in another specific aspect relates to methods for inducing increased levels of ubiquilin to reduce aggregation of polyglutamine expansion proteins known to cause cell toxicity and cell death in subjects suffering from neurodegenerative diseases, such as Huntington's and Alzheimer's diseases.

2. Background of the Related Art

The genes encoding the presenilin (PS) proteins along with β-amyloid precursor proteins (APP) are mutated in early-onset Alzheimer's disease and thus determining their function or dysfunction in normal and disease states is important. Interestingly, presenilins are believed to constitute the catalytic component of the γ-secretase complex implicated in the intramembrane proteolysis of β-amyloid precursor protein (APP) to release Aβ into the extracellular space [1-4].

At least three other components, including Nicastrin, Aph-1 , and Pen-2, are required for proper trafficking, processing, and stability of the PS proteins [1-3, 5-13]. Nicastrin is predicted to be a type 1 transmembrane protein that exists in two forms, an immature unglycosylated form and a mature highly glycosylated form which preferentially interacts with the extreme COOH-terminus PS [14,15]. During γ-secretase assembly, Nicastrin and Aph-1 form a subcomplex that is thought to stabilize PS [6, 11]. Aph-1 not only stabilizes PS, but it also stabilizes Nicastrin and hence is thought to act as a scaffolding protein for the γ-secretase complex mediating the trafficking of PS, Nicastrin, and Aph-1 to the Golgi [5, 6, 11, 16]. Pen-2 is required for endoproteolytic processing of PS meaning that when Pen-2 levels are down-regulated, build-up of full-length PS is observed [13]. Similarly, when PS levels are down-regulated, Pen-2 is destabilized and its cellular level decreases [17]. It therefore appears that Pen-2 and PS fragment levels are regulated in concert. While the overall cellular role of the four γ-secretase components are being actively studied; one thing is certain, all are required to reconstitute γ-secretase activity and mediate cleavage of APP in yeast [1]. Perhaps more importantly, γ-secretase is critical to the cleavage of other transmembrane proteins including ErbB4, Notch, D- and E-cadherins, LRP, CD44 and Syndecar-3, many of which play critical roles in development [18-20]. Thus inhibition of γ-secretase is a difficult target for drug design given its multitude of cellular roles, thus it would be beneficial to determine compounds that regulate the levels of its components could prove useful [3, 21-23].

PSs are present in cells as full-length (FL) proteins and as two fragments, termed the NH,-terminal fragment (NTF) and COOH-terminal fragment (CTF) derived by the endoproteolytic cleavage of the protein in the large loop region of the proteins by an unknown protease activity, called presenilinase [24-27]. Endoproteolytic cleavage sites are heterogeneous suggesting that either one protease can cleave at multiple sites or multiple proteases are responsible for PS cleavage [28-31]. In fact some γ-secretase inhibitors are also effective at preventing endoproteolysis, however not all are effective suggesting that the mechanism responsible for APP cleavage is not the same for PS cleavage, namely that the two conserved aspartate residues proposed to be the active sites for y-secretase are not responsible for the autoproteolysis of PS [28]. Pepstatin A, which is not a y-secretase inhibitor, but instead functions as an acidic protease inhibitor appears to be the most potent inhibitor of endoproteolysis known to date [32]. Others have reported the involvement of the proteasome in PS cleavage, which is an interesting proposal given the recent reports that the proteasome has endoproteolytic activity indicating that it has functions other than in protein degradation [29, 33].

Fragments are proposed to be the functional γ-secretase PS form and thus understanding their regulation is important. They occur in roughly 1:1 stoichiometry, meaning NTF and CTF levels are approximately the same, and their levels are saturable, meaning that when FL PS is overexpressed, a concomitant increase in NTF and CTF levels is not observed [24-26]. Instead, their levels are tightly regulated and excess fragments are rapidly degraded [34, 35]. While PS1 NTF and CTF interact in vivo and co-immunoprecipitate, PS 1 and PS2 fragments do not interact with each other and therefore do not form mixed complexes [34, 36]. This is not surprising given PS1 and PS2 display different cofractionation patterns [37]. However, fragment production and maintenance is more complicated than simple endoproteolysis, both the NTF and CTF are subject to different regulation. The PS1 CTF is phosphorylated by PKC and GSK-313, which appear to regulate CTF levels, but not PS1 NTF levels [38, 39].

Complicating the issue more, the PS2 NTF is phosphorylated by casein kinases while the CTF is subject to further cleavage by caspases demonstrating further not only the differences in NTF and CTF regulation, but also in PS1 and PS2 regulation [39, 40]. Clearly fragment production and regulation is a complicated matter worthy of understanding since they are likely to be the functional PS form and PS interactors that modulate fragment levels could be a potential drug design target.

The identification and characterization of Ubiquilin-1 (UBQLN1), a PS-interacting protein was described by several of the present inventors in U.S. patent application Ser. No. 10/293,000. It was found that ubiquilin has an ubiquitin-like domain (UBL) at its N-terminus and an ubiquitin-associated domain (UBA) at its C-terminus. In relation to PSs, UBQLN and PS partially colocalize in cells and UBQLN overexpression increases synthesis of FL PS proteins and inhibits degradation of ubiquitinated forms of PS proteins [43,48]. Notably, a genetic screen of brain tissue from patients with AD recently identified a strong linkage between UBQLN1 and AD [44]. To date, it is the only other known potential risk factor for late onset AD besides APOEε4. Ubiquilin-2 (UBQLN2) is 72% identical to UBQLN1 except that it has a collagen-like motif in its C-terminus, which could be involved in intra-cellular signaling [45]. UBQLN proteins are somehow linked to the ubiquitin-proteasome pathway of protein degradation since both the UBL and UBA of UBQLN have been shown to bind the S5a subunit of the proteasomal cap [46-47]. Further supporting its interaction with the proteasome, UBQLN was found not only to interact with E6AP E3 ubiquitin ligase, but also to partially fractionate with the proteasome [45].

Thus, it would be beneficial to determine the action of ubiquilin relative to the interaction with the Presenilins and/or modification of γ-secretase components.

Huntington's disease (HD) is an autosomal-dominant age-related neurological illness that is characterized by choreic movements, severe behavioral and emotional disturbances, and cognitive decline. It is believed that HD is caused by an abnormal polyglutamine (polyQ) expansion within the protein huntingtin (Htt) and is characterized by the aggregation of Htt into microscopic intracellular deposits called inclusion bodies (IBs) and by the death of striatal and cortical neurons. The duration of the disease usually lasts about 15-20 years, ultimately resulting in death [1a]. Currently, there is no effective treatment to prevent or cure HD.

Specifically, the expansion of a trinucleotide sequence (CAG) residing in exon 1 of the gene encoding huntingtin protein [2a] is believed to be the cause of HD. The expanded CAG repeats are translated into a stretch of polyglutamines (polyQ), which in nonaffected individuals ranges from between 14 to 34 glutamines, to the pathological fully-penetrant form which consists of greater than 40 repeats. To date, at least eight other neurological disorders are also known to be caused by an expansion of polyglutamine tracks, and these include, dentatorubral-palidoluysian atrophy (DRPLA), spinal and bulbar muscular atrophy (SBMA) and spinocerebella ataxias (SCAs) 1-3, 6, 7 and 17. Apart from all these diseases containing an expanded polyglutamine tract, the translated proteins in the nine disorders are otherwise unrelated in sequence, strongly suggesting that the expanded polyglutamine track is responsible for causing disease. Indeed expression of an artificial protein composed almost entirely of polyglutamine repeats, or introduction of an expanded polyglutamine stretch in an otherwise completely normal protein, is sufficient to induce neurodegeneration [3a ,4a].

Several theories have been proposed for the mechanism by which expanded polyglutamine tracts cause disease [5a-9a]. These theories include the reasoning that after polyglutamine repeats reach a certain threshold there is a greater propensity to aggregate, either with themselves or with other proteins, and that the aggregates cause a toxic-type function. By contrast, others have argued that aggregates are not toxic, but might, in fact, be protective [10a].

Regardless of the exact mechanism by which polyglutamine repeats cause disease it is clear that most polyglutamine-associated diseases display an inverse correlation between the length of polyglutamine repeats and the age of onset and severity of disease. However, this correlation is less obvious with shorter polyglutamine repeats, as was demonstrated by Wexler and colleagues who studied the age of onset of HD in a large population of HD-affected individuals in the Lake Maracaibo region of Venezuela [12]. The Wexler group demonstrated that age of onset of HD varied more considerably in individuals with shorter number of polyglutamine repeats in the huntingtin protein than those with longer repeats. These findings led the authors to propose that other unknown genetic and environmental factors most likely influence the age of HD, at least for those individuals with short polyglutamine tracts in Htt.

The present inventors became interested in the possibility that ubiquilin-1, a protein identified and described in copending U.S. patent application Ser. No. 10/293,000, which was shown to be present in neuropathological lesions in Alzheimer's and Parkinson's diseases, might be involved in regulating HD pathogenesis. Ubiquilin-1 has an ubiquitin-like domain (UBL) at its N-terminus and an ubiquitin-associated domain (UBA) at its C-terminus and ubiquilin-2 (UBQLN2) is 72% identical to ubiquilin-1 except that it has a collagen-like motif in its C-terminus, which could be involved in intra-cellular signaling. Thus, it would be beneficial to determine the action of ubiquilin or fragments thereof relative to the interaction with polyglutamine-containing proteins.

### BRIEF SUMMARY OF THE INVENTION

The present invention relates to ubiquilin, as well as to methods, formulations and techniques for employing ubiquilin, and nucleotide sequences encoding ubiquilin.

The present invention in one aspect relates to a method for decreasing fragmentation of full length presenilin 1 and/or 2 proteins; the method comprising
introducing an expression vector to a host cell that expresses presenilin 1 and/or 2, wherein the expression vector comprises a nucleotide sequence encoding ubiquilin;
maintaining the transformed host cell under biological conditions sufficient for expression and accumulation of the ubiquilin in the host cell; and
measuring the level of presenilin 1 and/or 2 fragments relative to a host cell not expressing increased levels of ubiquilin.

Preferably, the expression vector comprises a nucleotide sequence that encodes polypeptides comprising the amino acid residue sequence of SEQ ID NOs: 2 or 4 (amino acid sequences of ubiquilin 595 and 589, respectively) or a fragment thereof, or variant having at least 90% homology comprising a thereof that has the same functional activity of ubiquilin.

In a preferred embodiment the nucleotide sequence comprises SEQ ID NO 1 or 3, or sequence complementary to such sequences or hybridizes thereto under stringent hybridization conditions.

In another aspect, the present invention provides for a method for increasing full length presenilin 1 and/or 2 proteins by reducing fragmentation of full length presenilin 1 and/or 2 proteins, the method comprising;
introducing an expression vector to a host cell that expresses presenilin 1 and/or 2, wherein the expression vector comprises a nucleotide sequence encoding ubiquilin or a functional fragment thereof;
maintaining the transformed host cell under biological conditions sufficient for expression and accumulation of the ubiquilin in the host cell; and
measuring the level of full length presenilin 1 and/2 relative to a host cell not expressing increased levels of ubiquilin.

In yet another aspect, the present invention relates to a method for decreasing levels of Pen-2 and/or Nicastrin in a cell, the method comprising increasing levels of ubiquilin in the cell that expresses Pen-2 and/or Nicastrin.

In a still further aspect, the present invention relates to a method for reducing catalytic γ-secretase enzyme in a cell, the method comprising introducing a sufficient amount of ubiquilin to reduce the endoproteolysis formation of presenilin 1 and/or 2 fragments.

The present invention relates to the discovery that ubiquilin proteins bind and/or interact with polyglutamine-containing proteins and increasing levels of ubiquilin protects cells and animals from Htt-polyglutamine-induced toxicity and cell death.

The present invention relates to a method for decreasing cell death in a host cell exhibiting aggregation of polyglutamine-containing proteins, the method comprising;
introducing an expression vector to a host cell comprising a nucleotide sequence encoding ubiquilin in an amount to overexpress ubiquilin; maintaining the transformed host cell under biological conditions sufficient for expression and accumulation of the ubiquilin in the host cell, wherein overexpression of ubiquilin reduces sensitivity of cell to stress induced by expanded polyglutamine proteins.

Preferably, the expression vector comprises a nucleotide sequence that encodes polypeptides comprising the amino acid residue sequence of Ubiquilin, or variants having at least 90% homology and having the same functional activity of ubiquilin, or fragments thereof.

Sequences useful in such respect include SEQ ID NOs: 5, 7, 9, 13, 15 and 17.

In a preferred embodiment the nucleotide sequence comprises SEQ ID NOs: 5, 7, 9, 13, 15 or 17, or a nucleotide sequence having at least 95% identity or complementary to such sequences, wherein the expressed protein has the functional ability to reduce cell death in transformed cells expressing increased levels of ubiquilin.

In another aspect, the present invention provides for determining the effectiveness of ubiquilin in reducing polyglutamine expansion in a host cell, the method comprising:
introducing an expression vector to a host cell comprising a nucleotide sequence encoding ubiquilin or a fragment thereof having the same functional activity;
maintaining the transformed host cell under biological conditions sufficient for expression and accumulation of the ubiquilin in the host cell; and
measuring the level of cell death in the host cells relative to a host cell not expressing increased levels of ubiquilin.

In yet another aspect, the present invention relates to the use of an expression vector encoding for a ubiquilin protein or variant thereof having deletions or substitution but maintaining the functionality of ubiquilin, in a medicament for the treatment of neurological disorder including HD.

Other features and advantages of the invention will be apparent from the following detailed description, drawings and claims.

### BRIEF DESCRIPTION OF THE DRAWINGS

FIG. 1. Differential modulation of FL and PS protein fragments by UBQLN-1 in HEK293 inducible cell lines. (A) PS 1 NTF and CTF levels were analyzed in PS 1 cells lines that were either not induced (lanes 1 and 3) or induced with PonA (lanes 2 and 4) and either left untransfected (lanes 1 and 2) or transfected with UBQLN1 cDNA (lanes 3 and 4). Equivalent amounts of protein lysates were separated by 10% SDS-PAGE and immunoblotted with anti-PS1 NTF antibody and anti-PS1 loop antibody. The filter was then reprobed with anti-ubiquilin antibody to ensure overexpression and anti-actin to ensure equal loading. Overexpression of UBQLN1 decreases PS1 NTF and CTF levels (lanes 3 and 4). Densitometric analysis was used to quantify the extent of reduction. Each experiment was repeated more than three times.

(B) Same experiment as described in (A) except PS2 inducible cells were used to analyze PS2 NTF and CTF levels. Like PS1 fragments, UBQLN1 also decreases PS2 fragment levels. The nature of the 40 kDa band is unknown however, it appears to be related because a decrease in its levels are also observed upon UBQLN overexpression.

(C) Endogenous PS fragment levels were analyzed in HEK293 cells that were transfected with increasing amounts of UBQLN1 or UBQLN2 expression plasmids. Equivalent amounts of protein lysates were separated by 10% SDS-PAGE and immunoblotted with anti-PS1 NTF antibody. The filter was then reprobed with anti-ubiquilin antibody to ensure overexpression of UBQLN and with an anti-actin antibody to ensure equal protein loading. Overexpression of UBQLN decreases PS1 NTF levels and the extent of reduction was quantified again by densitometric analysis. Graph is quantification of untransfected and 8 pg UBQLN cDNA plasmid transfected lanes.

FIG. 2. Potential mechanisms by which UBQLN decreases PS fragment levels.

(A) FL PS is cleaved into its fragments. The PS fragments then interact with UBQLN which, in turn, enhances their rapid degradation through its ability to interact with the proteasome.

(B) UBQLN interacts with FL PS which prevents access of the presenilinase to the cleavage site thus blocking endoproteolysis.

FIG. 3. Evidence that overexpression of UBQLN does not facilitate rapid degradation of PS fragments. (A) PS1 cells lines that were either not induced (lanes 1 and 3) or induced with PonA (lanes 2 and 4) and either left untransfected (lanes 1 and 2) or transfected with UBQLN1 expression plasmids (lanes 3 and 4) were treated for 7 h with MG132 after which protein lysates were collected and analyzed by immunoblotting. Even after MG132 treatment, PS1 NTF and CTF levels remain reduced in transfected cells (compare lane 3 to 1 or lane 4 to 2), indicating that UBQLN does not decrease PS fragment levels by increasing the rate of turnover of the fragments. For clarification, if UBQLN did increase the turnover rate then by blocking degradation, turnover would be stopped and PS fragment levels would be similar, or increase, relative to that of untransfected cells. However, this was not the case, thus UBQLN does not increase turnover rate and thus must work by some other mechanism. Densitometric analysis was used to quantify the extent of reduction. Each experiment was repeated more than three times and similar trends were observed.

(B) Same experiment and results as described in (A) except that PS2 inducible cell lines were used to examine PS2 NTF and CTF levels. Again, since fragment levels remain reduced in UBQLN transfected cells, UBQLN overexpression does not decrease PS fragment levels by enhancing their turnover (compare lane 3 to 1 or lane 4 to 2). UBQLN exerts greater effects on PS2 probably because it interacts more strongly with PS2, see [43].

FIG. 4. UBQLN prevents endoproteolysis by slowing the rate of fragment production.

(A) Time course experiment using ponA-induced PS2 cell line cultures transfected with or without UBQLN1 expression plasmids were treated with 100 mM cycloheximide for 0-6 hours. Equal amounts of protein lysate were separated by 8.5% SDS-PAGE and subsequently immunoblotted with anti-UBQLN antibody. Transfected cells reveal a 4-fold increase in UBQLN1 levels. Next, the protein lysates were immunoblotted using anti-PS2 NH,-terminal antibody to detect FL and NTF PS2 levels, as indicated. Note both FL PS2 turnover and NTF production levels in transfected versus untransfected cell lysates. NTF levels of PS2 are significantly reduced in UBQLN1 transfected cells and the rate of NTF production is slowed.

(B) Same experiment as (A) except FL PS expression was not induced using PonA. Again overexpression of UBQLN not only decreases NTF levels, but also slows the rate of PS2 NTF production.

(C) Graphs showing relative levels of PS2 polypeptides in PS2 inducible cell lysates after densitometric analysis of the immunoreactive bands shown in panels A and B. The levels were calculated by normalization of the signals relative to that in Lane 1. The Microsoft Excel program was used to produce a best-fit line for each graph. UBQLN increases FL PS2 levels dramatically. However, this is completely opposite of NTF levels. UBQLN not only decreases NTF levels at every time-point in the experiment, but it also slows the rate of NTF production, as determined by measuring the slope of each line. Similar trends were observed for the CTF (data not shown).

FIG. 5. UBQLN knock-down leads to increased PS fragment levels

(A) 2% agarose gel electrophoresis of PCR products obtained after RT-PCR experiment. Lane 2 verifies UBQLN1 mRNA levels of untransfected cells while lanes 3 and 4 exhibit knockdown of UBQLN1 mRNA when transfected with UBQLN1-2 siRNA or both UBQLN1-2 and UBQLN2-l siRNAs, respectively. Lane 5 verifies UBQLN2 mRNA levels of untransfected cells while lanes 6 and 7 demonstrate knockdown of UBQLN2 mRNA when transfected with UBQLN2-1 siRNA or both UBQLN1-2 and UBQLN2-1 siRNAs. Both siRNA constructs are effective at reducing the UBQLN mRNA. An immunoblot showing UBQLN protein levels after siRNA transfection demonstrates a 72% and 46% reduction in UBQLN1 and UBQLN2 levels, respectively, was achieved (bottom panel).

(B) Fragment levels in PS2 inducible cells transfected with increasing amounts of the siRNAs most effective at knocking-down UBQLN protein levels were analyzed by 10% SDS-PAGE and subsequent immunoblot analysis. Decreased UBQLN levels leads to increased PS2 CTF levels (top panel) demonstrating that UBQLN protein reduction is associated with an increase in PS fragment accumulation. The filter was reprobed with anti-actin antibody to ensure equal protein loading since changes in CTF levels are subtle. Anti-UBQLN reprobing illustrates the decrease in UBQLN1 levels although UBQLN2 levels were not resolved. Nonetheless, consistent decreases in UBQLN2 levels are observed.

(C) PS2 inducible cells were transfected with 25 nM final cont. of UBQLN1-2 and UBQLN2-1 siRNAs. Cell lysates were collected and analyzed after 72 h. Both PS1 NTF (top panel) and PS2 NTF (middle panel) levels are increased in the transfected cells compared to cells transfected with nonsense siRNA or mock transfected cells. The filter was re-probed with anti-actin antibody to ensure equal protein loading. Re-probing with an anti-UBQLN antibody demonstrates that UBQLN protein levels are reduced in the PS2 stable cell line (bottom panel).

(D) Normal HEK293 cells were transfected with 30 nM final cont. of UBQLN1 and UBQLN2 SMARTpool siRNAs. Cell lysates were collected and analyzed after 48 h. Endogenous levels of PS1 NTF (top panel) and PS2 NTF (middle panel) levels both are increased in the transfected cells compared to nonsense transfected cells. The membrane was re-probed with anti-actin antibody to ensure equal protein loading and anti-UBQLN antibody to demonstrate that UBQLN levels are reduced.

FIG. 6. UBQLN affects Pen-2 and Nicastrin levels.

(A) Examination of Nicastrin, Aph-1 and Pen-2 levels in PS1 cells lines that were either not induced (lanes 1 and 3) or induced with PonA (lanes 2 and 4) and either left untransfected (lanes 1 and 2) or transfected with UBQLN1 cDNA (lanes 3 and 4). Equivalent amounts of protein lysates were separated by 10% SDS-PAGE and immunoblotted with the appropriate antibodies. The filter was then reprobed an anti-actin to ensure equal loading. UBQLN overexpression decreases Pen-2 and Nicastrin levels.

(C) Densitometric analysis was used to quantify the extent of reduction. Each experiment was repeated more than three times.

(D) Normal HEK293 cells were transfected with 30 nM final COW. of UBQLN1 and UBQLN2 SMARTpool siRNAs. Cell lysates were collected and analyzed after 48 h. Endogenous levels of Nicastrin (top panel) and Pen-2 (middle panel) both increase following UBQLN siRNA transfection compared to Nonsense transfected lysates. The membrane was re-probed with anti-actin antibody to ensure equal protein loading and anti-UBQLN antibody to demonstrate that UBQLN levels are reduced.

FIG. 7. Implication that the proteasome is involved in PS2 cleavage.

(A) PS1 and PS2 inducible cells were treated with various classes of proteasome inhibitors for 16 hours. Afterwards, cell lysates were collected, separated by SDS-PAGE, and analyzed by immunoblotting. PS1 NTF levels and PS2 NTF levels were reduced by treatment with all of the different inhibitors albeit to varying extents. FL PS levels are extremely low in these cells and hence are not able to be observed.

(B) PS2 inducible cells were treated continuously with MG132 or MG262 or the drugs were washed from half the cultures after 7 hours of incubation with the drugs. Immediately after washing, CHX was added to all of the cultures to inhibit protein synthesis. Lysates were collected at two hour time points thereafter and the PS fragment levels were analyzed by SDS-PAGE and subsequent immunoblotting. A significant increase is seen in PS2 NTF levels as the proteasomes inhibition is relieved suggesting the proteasome may be involved in PS endoproteolysis (top panel, right). Similar trends were observed for MG262 treatment (second panel, right). Please note that because PS2 expression was not been induced with PonA, the FL protein is not detectable. The filter was reprobed with anti-p27 antibody to monitor recovery of proteasome activity. p27 has a half-life of two hours; therefore once proteasome inhibition is relieved the level of p27 begins to decline (third panel, right), whereas under constant proteasome inhibition its levels remain steady (third panel, left), serving as an important control. Finally, the filter was reprobed for actin to demonstrate equal protein loading.

(C) Densitometric analysis was used to quantify the extent of increase in PS2 NTF levels in conditions of constant proteasome inhibition and upon removal of proteasome inhibition. At time=0 the NTF levels in the washed sample is equal to that of MG132 treated sample; however, NTF levels quickly begin to climb as proteasome activity recovers, whereas in the MG132 treated samples PS2 NTF levels stay steady.

FIG. 8. This Figure shows that overexpression of GFP-Htt-Exon1-polyQ constructs in HeLa cells leads to a polyglutamine-length dependent increase in cell death and Trion-X100 insolubility.
A. Immunoblot showing anti-GFP immunoreaction in HeLa cell lysates, 24 hours after transfection with 10 □g plasmid DNA corresponding to the following constructs: lane 1, mock transfected; lane 2, pEGFP; lane 3, pEGFP-HttExon-1polyQ(28); lane 4, pEGFP-HttExon-1polyQ(55); and lane 5, pEGFP-HttExon-1polyQ(74).
B. Cell death seen in parallel dishes of HeLa cells 24 hours after transfection with the constructs described above after normalization to the level of mock transfected cells.
C. Immunoblots showing GFP-immunoreactivity after biochemical fractionation of transfected HeLa cells into Triton-X100 soluble and pellet fractions. Upper panel is the anti-GFP immunoblot and the lower panel the anti-lamin A/C immunoblot of equal proportions of the fractionated proteins seen in cells 24 hours after transfection with the different constructs described in A.
D. Densitometric quantification of anti-GFP immunoreactive bands depicting the relative amount of GFP-immunoreaction found in the pellet compared to that the total (soluble and pellet fractions combined).

FIG. 9. This Figure shows that overexpression of ubiquilin is associated with a dose-depended increase in GFP-HttpolyQ74 protein accumulation, reduction in cell death and Triton-X100 insolubility.
A. HeLa cells were co-transfected with a constant amount (5 μg) of pEGFP-HttExon-1polyQ(74) expression construct and increasing amounts of cDNA ubiquilin-1 expression construct as indicated.
B. Cell death seen in parallel dishes of HeLa cells 24 hours after transfection with the constructs described above after normalization to the level of mock transfected cells.
C. Immunoblots showing GFP-immunoreactivity after biochemical fractionation of parallel sets of HeLa cells transfected with the constructs described in (A) into Triton-X100 soluble and pellet fractions.
D. Densitometric quantification of anti-GFP immunoreactive bands depicting the relative amount of GFP-irnmunoreaction found in the pellet compared to that the total (soluble and pellet fractions combined).

FIG. 10. This Figure shows that overexpression of ubiquilin-1 reduces polyglutamine inclusions and cytotoxicity in mouse primary neuronal cultures.
A. Representative images of mouse neurons transfected with the plasmids indicated. Mouse cortical neurons (14 days in vitro) were transiently transfected with pEGFP (3 μg each well, a), or pEGFP-HttExon-1polyQ(74) alone (3 μg each well, b), or cotransfected with both pEGFP-HttExon-1polyQ(74) and ubiquilin-1 cDNA with the ratio indicated (3 μg of pEGFP-HttExon-1polyQ(74) plus 1.5 μg or 3 μg of ubiquilin-1 cDNA; c, d).

B. Overexpression of ubiquilin-1 reduces formation of GFP-HttPolyQ(74) inclusions in cultured mouse neurons. The results shows are the mean of the number of cells with eye-detectable aggregates either in the cell body or neurites±SD.
C. Overexpression of ubiquilin-1 reduces GFP-HttPolyQ(74)-induced cell death in cultured mouse neurons. 30 hours following transfection, the cultures were stained with both Hoechst 33342 and propidium iodide (PI) and then subjected for fluorescent microscopy analysis. PI positively stained cells were treated as dead cells and only the green (GFP-positive) cells were included in the cell death analysis. Results are mean±SD.

FIG. 11. This figure shows that the overexpression of ubiquilin-1 reduces polyglutamine cytotoxicity in stable cell lines expressing expanded GFP-Httpolyglutamine fusion proteins.
A. GFP immunoblot (upper panel) and actin immunoblot (lower panel) of equal amounts of protein lysate from cell lines stably expressing GFP, or GFP-fused to Htt exon 1 containing 28Q, 55Q, or 74Q. The 28Q-2 and 74Q-3 were used in most of our studies. The asterisk corresponds to a non-specific band that is detected by the GFP antibody.
B. Representative fluorescent images of GFP-HttPolyQ(28)-2 and GFP-HttPolyQ(74)-3 stable cell lines. Note that GFP fluorescence is located in both the cytoplasm and the nucleus, but that compact foci are only seen in the GFP-HttPolyQ(74)-3 cells.
C. Cells expressing expanded polyglutamine proteins are more sensitive to H₂O₂. The GFP-HftPolyQ(28)-2 and GFP-HttPolyQ(74)-3 stable cell lines were exposed to 200 μM concentration of H₂O₂ and after 5 hr, the cells were stained with both Hoechest 33342 and PI. The cells stained by PI represent the dead cells whereas all the nuclei are identified by Hoechst 33342 staining.
D. Graph showing percentage of cell death after the cells were exposed to different concentrations of H₂O₂ for 5 hr. Percentages of cell death calculated by determining the ratio of PI-positively stained cells to that of Hoechst-stained cells. There was a significant difference in cell death upon exposure of GFP-HttPolyQ(28)-2 and GFP-HttPolyQ(74)-3 to 100 μM, 150 μM, or 200 μM of H₂O₂ (p<0.001).
E. Representative images showing that overexpression of ubiquilin-1 protects GFP-HttPolyQ(74)-3 expressing cells against serum withdrawal-induced cell death. Cells stably expressing GFP-HttPolyQ(28) or GFP-HttPolyQ(74) were transiently transfected with or without (mock transfection) plasmids encoding ubiquilin-1 by the calcium phosphate coprecipitation method. Following DNA-calcium phosphate transfection, the cultured were maintained in serum free medium for 5 hours and then maintained in medium containing serum for the remainder of the experiment. The images shown were taken at 30 hours after transfection.
F. Immunoblot of equal amounts of protein lysate from GFP-HttPolyQ(28)-2 and GFP-HttPolyQ(74)-3 cell lines transfected without (mock transfection) or with a ubiquilin-1 expression construct. Note that the ubiquilin antibody recognizes both ubiquilin-1 (lower band) and -2 (upper band). The same membrane was reblotted for actin, as a loading control.
G. Graph showing ubiquilin-1 overexpression protects against serum withdrawal-induced cell death. Cell death was quantified as described in D above.
H. Representative images showing that ubiquilin-1 overexpression protects GFP-HttPolyQ(74)-3 expressing cells against serum withdrawal-induced cell death, in a dose-dependent manner. Cells stably expressing GFP-HttPolyQ(74) were transiently transfected without (a, mock transfection) or with an increasing amount of a cDNA encoding ubiquilin-1 (b, 1μg; c, 2 μg; d, 3 μg; e, 6 μg; f, 9 μg). After 5 hours of serum withdrawal and 30 hours following the transfection, the amount of cell death was analyzed by staining cells with both PI and Hoechst 33342.
I. Graph showing quantification of cell death in the experiments described in H.
J. Immunoblot showing overexpression of increasing amounts of ubiquilin-1 cDNA in the GFP-HttPolyQ(74)-3 cell line reduces GFP-aggregates trapped in the stacking gel after SDS-PAGE. The amount of ubiquilin-1 cDNA transfected is shown. Please note that both ubiquilin and GFP-HttPolyQ(74) are found in the stacking gel (probably because of binding to each other) and that these insoluble aggregates decrease with increasing ubiquilin-1 expression.
K. A filter retardation assay also demonstrates that overexpression of increasing amounts of ubiquilin-1 cDNA in the GFP-HttPolyQ(74)-3 cell line reduces GFP-aggregates. Top panel: 20 μg of cell lysates from either GFP-HttPolyQ(28)-2 cells which do not form aggregates, used as a control, or from GFP-HttPolyQ(74)-3 cells after either mock transfection or transfection of increasing amounts of ubiquilin-1 expression construct as indicated. The lysates were filtered through a cellulose acetate membrane and then probed with an anti-GFP antibody. Bottom panel: Quantification of the spot intensity determined in three independent experiments. The spot intensity from the 2, 3, and 6 μg of ubiquilin-transfected GFP-HttPolyQ(74)-3 cells are significantly lower than that of from corresponding cells mock-transfected or transfected with 1 μg of ubiquilin.

FIG. 12. This figure shows that RNA interference of ubiquilin in GFP-HttPolyQ(74)-3 cells leads to inhibition of cell proliferation, and promotes GFP-polyglutamine aggregation and cell death over time.
A. Immunoblots showing successful knockdown of ubiquilin protein levels by siRNAs. GFP-HttPolyQ(74)-3 cells were either mock transfected, or transfected with a combination of ubiquilin 1 and 2 SMARTpool siRNAs, or transfected with a non-target control SMARTpool siRNA. Cells were harvested 2, 3, or 4 days following the transfection and equal amounts of protein was immunoblotted for ubiquilin. Ubiquilin levels were downregulated to <10% of the normal levels 2 days after transfection and this low level of protein was maintained for at least until 4 days after transfection.
B. Ubiquilin knockdown is associated with decreased cell-proliferation. GFP-HttPolyQ(74)-3 cells, cultured in a 24-well plate at a low density, were either mock transfected or transfected with ubiquilin 1 and 2 or control siRNAs. The phase contrast shown, were taken just before transfection and 4 days after siRNA transfection.
C. Ubiquilin knockdown is associated in an increase in nuclear condensation/DNA fragmentation. GFP-HttPolyQ(74)-3 cells were transfected as siRNAs as described in A above, and stained with Hoechst 33342 on day-5 following transfection.
D. Graph showing quantification of condensed/fragmented in the experiments described in 5C. The asterisk indicates that there is a significant difference in nuclear condensation/fragmentation between the cells transfected with ubiquilin siRNA with either mock transfected cells or cells transfected with control siRNA (p<0.05).
E. TUNEL staining of GFP-HftPolyQ(74)-3 cells transfected with reagent vesicle alone (mock transfection), ubiquilin siRNAs, or control siRNA. 5 days following the transfection, cells were fixed and subjected to TUNEL staining.
F. Graph showing percentage of TUNEL positive cells seen in the experiments described in E.
G. Graph showing quantification of cell death in GFP-HttPolyQ(74)-3 cells, 5 days after transfection with the siRNAs, as described in B. The data shown represents the percentage of PI versus total Hoechst positive cells.
H. Ubiquilin knockdown in GFP-HttPolyQ(74)-3 cells is associated with increased caspase-3 activation. GFP-HttPolyQ(74)-3 cells were transfected with siRNAs, as describe in B, and 4 days after transfection equal amounts of protein lysate were immunoblotted for the cleaved (active) form of caspase-3. The immunoblot also shows a lysate of GFP-74Q cells treated with 1 □M staurosporine for 4 hr, as a positive control.
I. Graph showing flurogenic measurement of caspase-3 activity after ubiquilin knockdown in the GFP-HttPolyQ(74)-3 cell line. Experiment similar to H, but this time caspase-3 activity was determined by measuring the cleavage of a fluorescent caspase-3 substrate.
J. Ubiquilin knockdown increases the amount of GFP-74Q aggregates formed in the GFP-HttPolyQ(74)-3 cell line. Filter retardation assay was performed to detect HttPolyQ(74) aggregates. Equal amounts of lysates, diluted to different extents, prepared from cells 5 days after transfection with the siRNA described in B, were filtered through a cellulose acetate membrane and probed with an anti-GFP antibody.
Graph showing measurement of spot intensity of aggregates formed in the GFP-HttPolyQ(74)-3 cell line in three independent experiments as described in 5J.

FIG. 13. This figure shows that expression of polyglutamine expansions in C. elegans muscle results in length-dependent aggregate formation and motility defect.
A. Immunoblot showing anti-GFP immunoreaction in C. elegans protein extracts using 3- to 4-day-old animals expressing different lengths of GFP-HttpolyQ proteins: lane 1, wild-type C. elegans; lane 2, pGFP; lane 3, GFP-Htt(Q28); lane 4, GFP-Htt(Q55); lane 5, GFP-Htt(Q74). The lower panel is the α-actin immunoblot of the same blot shown above.
B. GFP fluorescence micrographs of young adult (3- to 4-day-old) C. elegans expressing different lengths of GFP-polyglutamine fusion proteins. Note that GFP fluorescence is mainly localized to the body wall muscle cells. Also, note that more compact foci form with increasing polyglutamine expression.
Higher magnification of showing the body wall of young adult C. elegans expressing different lengths of GFP-polyglutamine fusion proteins as described in
B.
C. Motility assay measured as body bends per minute in wild-type (N2) and various transgenic lines of adult C. elegans. Data are mean±SD for at least 12 animals of each type. Note that the rate of movement decreases with increasing length of polyglutamine expression.

FIG. 14. This figure shows that RNA interference of ubiquilin in C. elegans GFP-HttPolyQ expressing lines exacerbates the motility defect even further, whereas RNA interference of GFP rescues movement.
A. Downregulation of GFP using RNAi rescues motility defect caused by aggregates. Motility assay measured as body bends per minute of wild-type and transgenic lines expressing different lengths of polyglutamines (Q28 and Q74) grown on bacteria transformed with RNAi vectors for GFP (pPD128.1 10) with or without IPTG. Data are mean±SD for at least 12 animals of each type.
B. Downregulation of ubiquilin using RNAi exacerbates motility defect in transgenic lines. Quantitation of motility index for young adult animals (wild-type, Q28, Q72) grown on bacteria transformed with RNAi vectors for ubiquilin (L4440+ubiquilin) or empty vector, with or without IPTG as indicated. Data are mean±SD for at least 24 animals of each type as a percentage of control motility.
C. Representative image of HeLa cell expressing monomeric RFP-C. elegans ubiquilin fusion protein. HeLa cells were transfected with an mRFP-C. elegans ubiquilin plasmid construct and viewed under fluorescence microscopy after 24 hours.
D. Immunoblot of HeLa lysates showing specificity of C. elegans ubiquilin and mRFP antibodies. Panel 1(probed with anti-C. elegans ubiquilin antibody) and panel 2 (probed with anti-mRFP antibody): lane 1, untransfected control; lane 2, transfected with an mRFP-C. elegans ubiquilin plasmid construct. Note that both antibodies detected an 80 kDa band corresponding to mRFP-C. elegans ubiquilin fusion polypeptide.

FIG. 15. This figure shows that co-expression of ubiquilin diminishes aggregate formation and prevents the motility defect caused by GFP-Htt(Q55).
A-B. Fluorescence micrographs of three lines of young adult (3- to 4-day-old) C. elegans expressing GFP-Htt(Q55) and mRFP-ubiquilin (a-i) or GFP-Htt(Q55) only (j-1). The fluorescence micrographs for GFP and RFP in the coexpressing lines 1, 2, and 3 are shown in (a, d and g) and (b, e, and h), respectively, and the result of merging the GFP and RFP images is shown in (c, f, and i), respectively. Note that a, d, and g display different extents of more diffuse pattern of GFP fluorescence compared to j, k, and l.
C. Fluorescence micrographs of young adult C. elegans co-expressing GFP-Htt(Q55) and mRFP-ubiquilin. (a) fluorescence micrograph for GFP fluorescence in the coexpressing line 1; (b) corresponding fluorescence micrograph for RFP in the same animal. Note that co-expression of mRFP-ubiquilin diminishes aggregate formation of GFP-Htt(Q55). (c) fluorescence micrograph for GFP fluorescence in the coexpressing line 3; (d) corresponding fluorescence micrograph for RFP in the same animal; (e) merged image of (c) and (d). Small arrows indicate that GFP-Htt(Q55) and mRFP-ubiquilin co-localize at foci while arrowheads indicate lack of colocalization of ubiquilin with the GFP-polyglutamine fusion protein.
D. Motility assay measured as body bends per minute in three lines of young adult C. elegans expressing either GFP-Htt(Q55) alone or coexpressing GFP-Htt(Q55) with mRFP-uibiquilin. Data are mean±SD for at least 12 animals of each type. Note that co-expression of mRFP-ubiquilin prevents the motility defect, to different extents, typically caused by expression of GFP-Htt(Q55).

FIG. 16. This figure shows applicable nucleotide and amino acid sequences used in the practice of the present invention.

### DETAILED DESCRIPTION OF THE INVENTION

In order to facilitate review of the various embodiments of the invention and provide an understanding of the various elements and constituents used in making and using the present invention, the following terms used in the invention description have the following meanings.

Definitions

The tern “nucleic acid sequence,” as used herein, refers to an oligonucleotide, nucleotide, or polynucleotide, and fragments or portions thereof, and to DNA, cDNA or RNA of genomic or synthetic origin, which may be single- or double-stranded, and represent the sense or antisense strand.

The term “amino acid sequence,” as used herein, refers to an oligopeptide, peptide, polypeptide, or protein sequence, and fragments or portions thereof, and to naturally occurring or synthetic molecules.

The term “modulate,” as used herein, refers to a change in the activity of a polypeptide. For example, modulation may cause an increase or a decrease in protein activity, binding characteristics, or any other biological, functional or immunological properties of the polypeptide.

The term “substitution,” as used herein, refers to the replacement of one or more amino acids or nucleotides by different amino acids or nucleotides, respectively. Modifications and changes can be made in the structure of a polypeptide of the present invention and still obtain a molecule having like ubiquilin peptide characteristics. For example, certain amino acids can be substituted for other amino acids in a sequence without appreciable loss of peptide activity.

In making such changes, the hydropathic index of amino acids can be considered. The importance of the hydropathic amino acid index in conferring interactive biologic function on a polypeptide is generally understood in the art (Kyte, J. and R. F. Doolittle 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within .+−0.2 is preferred, those which are within .+−0.1 are particularly preferred, and those within .+−.0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e. with a biological property of the polypeptide. As detailed in U.S. Pat. No. 4,554,101, the following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); proline (−0.5.+−0.1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within .+−0.2 is preferred, those which are within .+−0.1 are particularly preferred, and those within .+−.0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 1, below). The present invention thus contemplates functional or biological equivalents of a peptide as set forth above.

TABLE 1

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

The term “functionally active,” as used, refers to a protein having structural, regulatory, or biochemical functions of a naturally occurring molecule.

The term “homology,” as used herein, refers to a degree of complementarity. There may be partial homology or complete homology. A partially complementary sequence that at least partially inhibits an identical sequence from hybridizing to a target nucleic acid is referred to using the functional term “substantially homologous.” The inhibition of hybridization of the completely complementary sequence to the target sequence may be examined using a hybridization assay (Southern or northern blot, solution hybridization and the like) under conditions of high to low stringency. A substantially homologous sequence or hybridization probe will compete for and inhibit the binding of a completely homologous sequence to the target sequence under conditions of low stringency.

The term “hybridization”, as used herein, refers to any process by which a strand of nucleic acid binds with a complementary strand through base pairing. A hybridization complex may be formed in solution or between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

As used herein, the term “stringent conditions” refers to conditions that permit hybridization between polynucleotide sequences and the claimed polynucleotide sequences. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

The term “transformed cell,” as used herein, is a cell into which has been introduced, by means of recombinant DNA technique, a DNA molecule encoding a protein of interest.

The term “transformation,” as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells that transiently express the inserted DNA or RNA for limited periods of time.

The present invention in one aspect thereof demonstrates that UBQLN decreases PS NTF and CTF levels by preventing endoproteolysis. It is further shown that the decrease in PS fragment levels modulated by UBQLN is accompanied by a decrease in Pen-2 and Nicastrin levels.

Experimental Procedures

Cell Culture

HeLa and HEK293 cells were grown in DMEM supplemented with 10% FBS. PS1 and S2 inducible cell lines were generated by transfection of the pERV-expressing HEK293 cell line (Stratgene, IaIolla, Calif.) with FL human PS2 cDNA under the control of ponasteroneA (PonA)-inducible cassette in plasmid pEGSH (Stratagene). Stable integration of the pEGSH plasmid was achieved by selection for hygromycin resistance. PS expression was induced by adding PonA (final cont. 10 pM) to the medium. For proteasome inhibition studies, cultures were treated with 40 uM MG132 (Calbiochem, San Diego, Calif.) for time periods as indicated. For protein inhibition studies by cyclohexamide, two sets of HEK293 stable cell lines were grown in 100 mM dishes, and one set was transfected with 20 ug FL UBQLN1 expression construct, and the other set was left untransfected. PonA was then added to half of the transfected and untransfected cultures and 16 hours later cyclohexamide (100 uM final conc.) was added to all of the cultures. Proteins lysates were prepared immediately after the addition of cyclohexamide and at 1 h intervals thereafter, as indicated.

Proteasome Studies

PS2 inducible cells were treated for 16 h with 10 uM clasto-lactacystin β-lactone, N-Acetyl-Leu-Leu-Nle-CHO (ALLN), 50 uM epoxomicin, 40 uM MG132, and 100 uM 10 PM MG262. Afterwards, lysates were collected and the fragment levels were analyzed by SDS-PAGE and immunoblotting.

PS2 inducible cells were grown in two sets on 100 mM dishes. Both sets were treated with MG132 or MG262 (also called Z-Leu-Leu-Leu-B(OH)₂, Boston Biocem). 7 h later, the drugs were washed off one set by rinsing twice with warm 1×PBS and replacing with fresh DMEM supplemented with 10% FBS, allowing the proteasomes to recover. Also at that point, cyclohexamide (final cont. 100 mM) was added to all dishes. Lysates were collected at two hour time points and the fragment levels were analyzed by SDS-PAGE and subsequent immunoblotting.

SDS-PAGE and Immunoblots

Preparation of protein lysates, SDS-PAGE and immunoblotting of proteins were described previously [43]. Primary antibodies used were rabbit anti-PS2 loop and rabbit anti-PS2 NH,-terminus, both raised to GST-PS2 fusion proteins [49]; rabbit anti-ubiquilin [43] which reacts with UBQLN1 and UBQLN2 polypeptides; rat anti-PS1 NH₂,-terminus (Chemicon International, Temecula, Calif.); rabbit anti-PS1 loop raised to GST-PS1 fusion proteins; goat anti-p27 and goat anti-actin (Santa Cruz Biotechnology, Santa Cruz, Calif.); mouse monoclonal anti-tubulin (Sigma-Aldrich, St. Louis, Mo.); rabbit anti-nicastrin (Abeam, Cambridge, Mass.), rabbit anti-Pen-2 (Zymed, San Francisco, Calif.); and rabbit anti-Aph-la (Covance, Berkeley, Calif.).

UBQLN RNAi Studies

siRNAs specific to either UBQLN1, named UBQLN1-1 and UBQLN1-2, UBQLN2, named UBQLN2-1 and UBQLN2-2, or a nonsense sequence were synthesized by Dharmacon RNA Technologies and were transfected into HeLa and HEK293 cells at 10, 15, and 25 nM final concentration using Mirus TKO transfection reagent. siRNA target sequences are as follows: UBQLN1-1 (AAGACCCCGAAGGAAAAGGAG), UBQLN1-2 (AACCUGGACAUCAGCAGUUUA), UBQLN2-1 (AACGCUUCAAAU CCCAAACCG), UBQLN2-2 (AAACCACGAGUCCUACAUCA G) and a nonsense sequence (AAATGAACGTGAATTGCTCAA). Cell lysates were collected and analyzed after 48 and 72 h. RT-PCR was conducted using Ambion's Cells-to-cDNA II kit. Basically, HeLa cells were transfected with the siRNAs, 48 h later the RNA was isolated and reverse transcribed. Next, the hcDNA was PCR amplified using UBQLN primers that were 200 bp apart. The PCR product was analyzed by 2% agarose gel electrophoresis.

Long-term UBQLN knockdown in the PS inducible cells was achieved using the Ambion's Silencer Express Kit to generate siRNA Expression Cassettes (SECs). The SECs were then cloned into Ambion's pSEC vectors and UBQLN protein reduction was analyzed by immunoblotting. The sequence targeted by the UBQLN1 SEC was AACAAATGCAGAATCCTGATA and the sequence targeted by the UBQLN2 SEC was AATCATCAAAGTCACGGTGAA.

Knockdown of UBQLN1 and UBQLN2 in wild-type HEK293 to study endogenous PS fragment levels and y-secretase component levels was achieved by using siRNA SMARTpools generated by Dharmacon RNA Technologies. These SMARTpools combine four different siRNAs that are identified as optimal sequences to achieve knockdown. The SMARTpools were transfected into HEK293 cells using DharmaFECT transfection reagent and protein levels were analyzed 48 hours post-transfection.

Results

Ubiquilin Decreases PS Fragment Levels

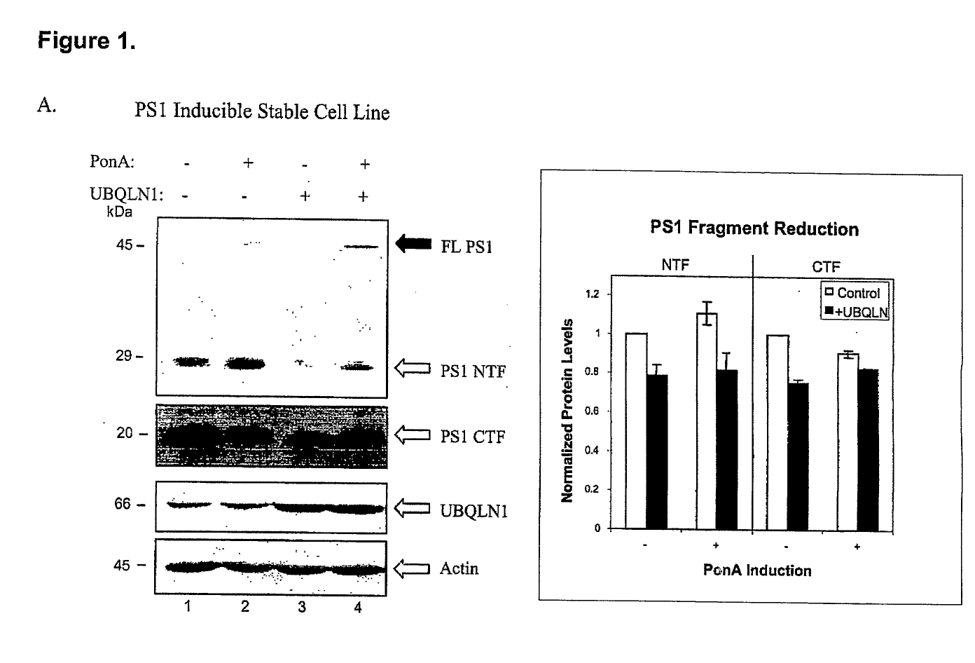

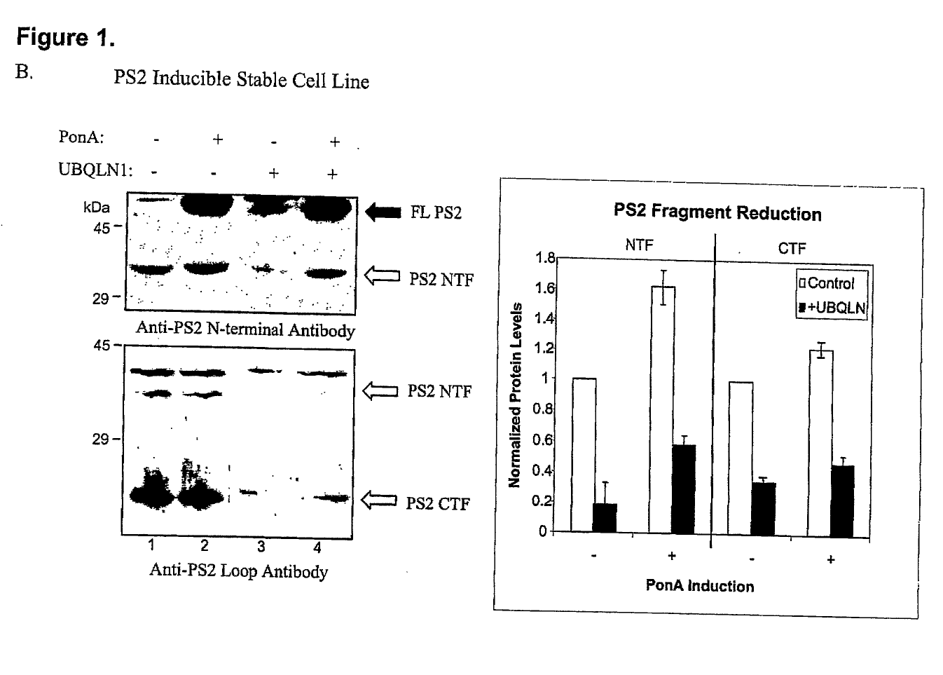

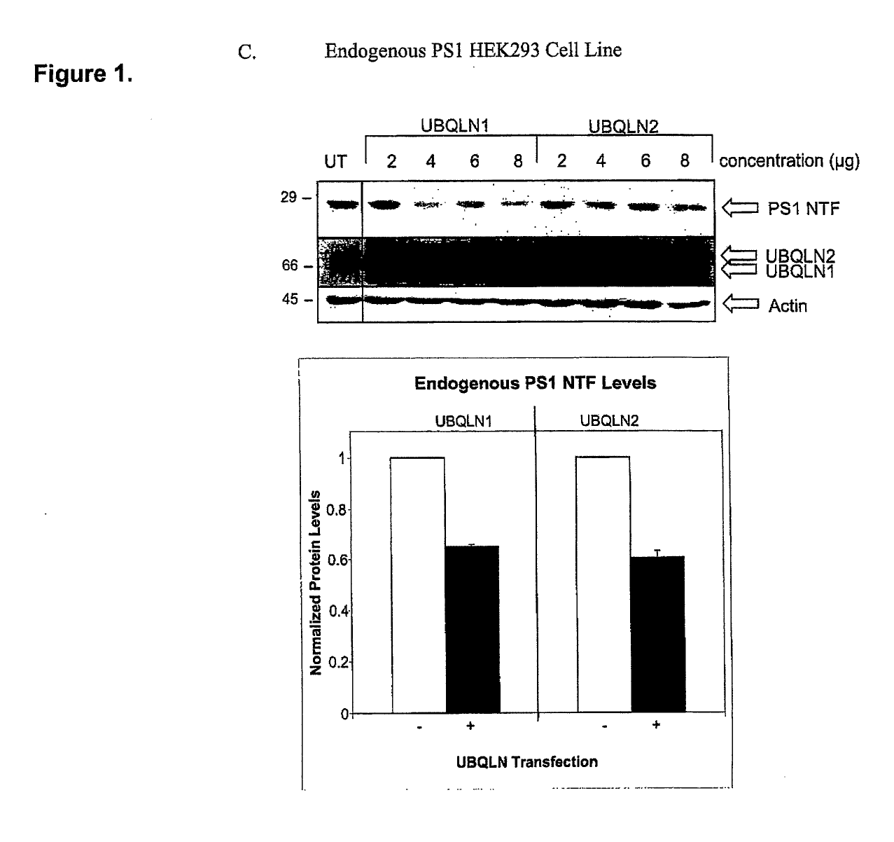

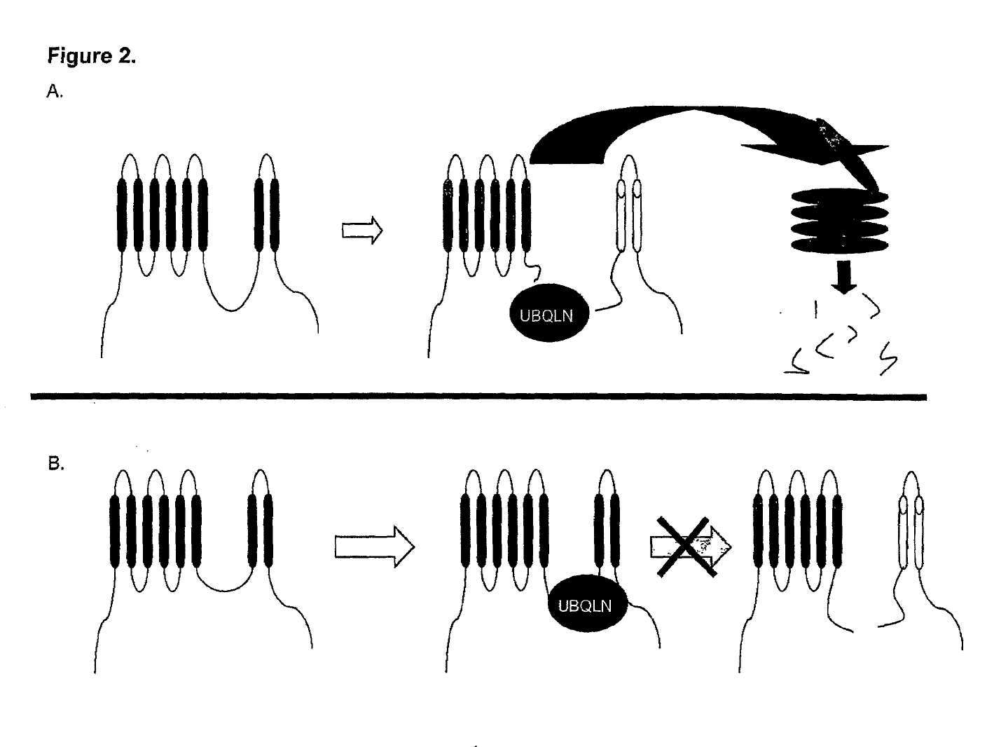

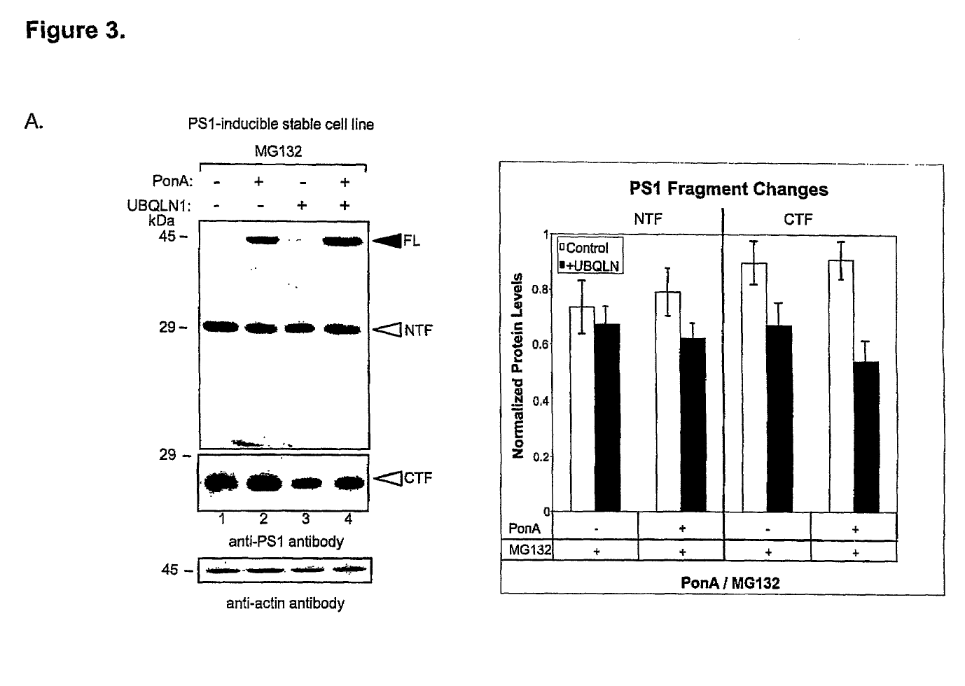

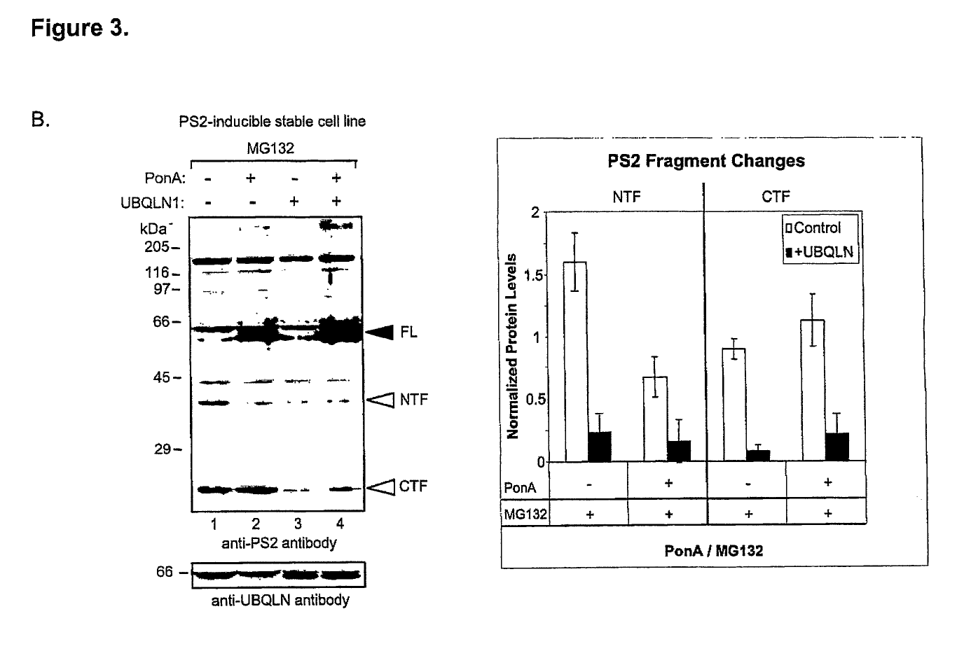

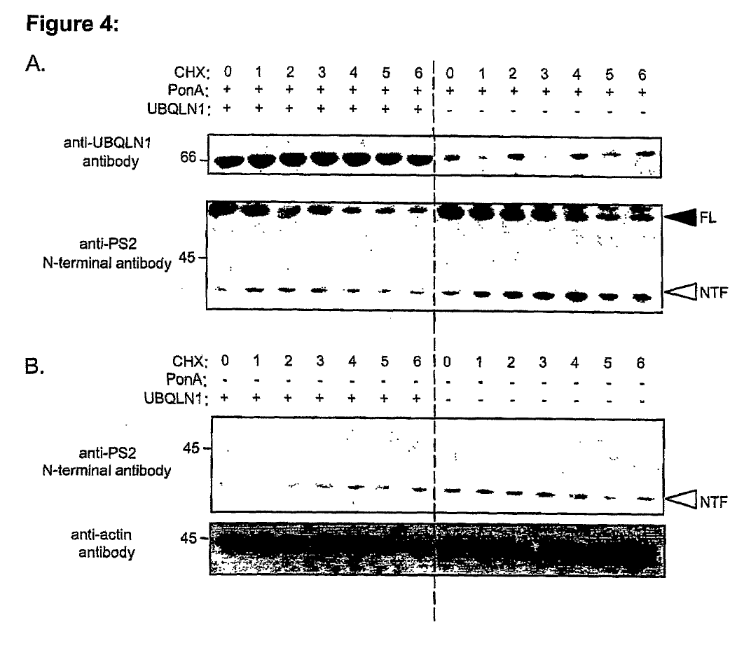

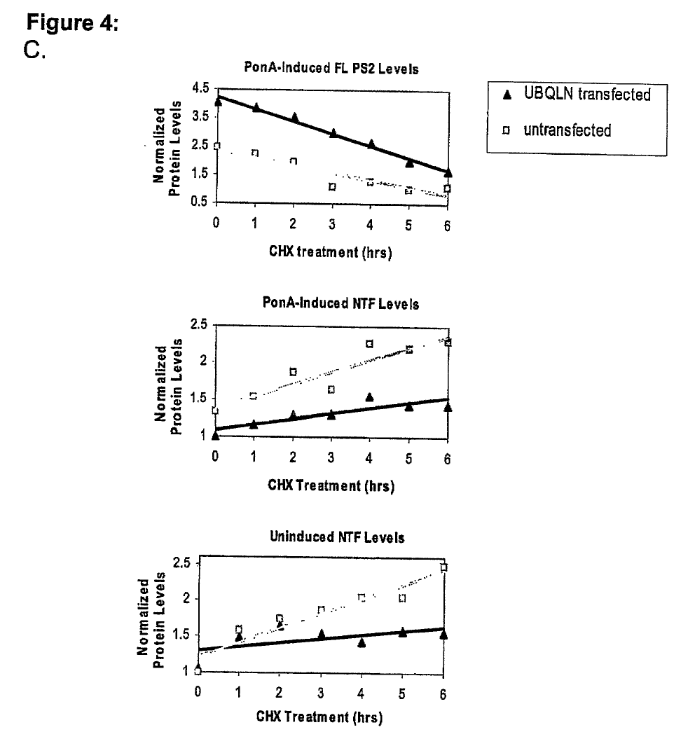

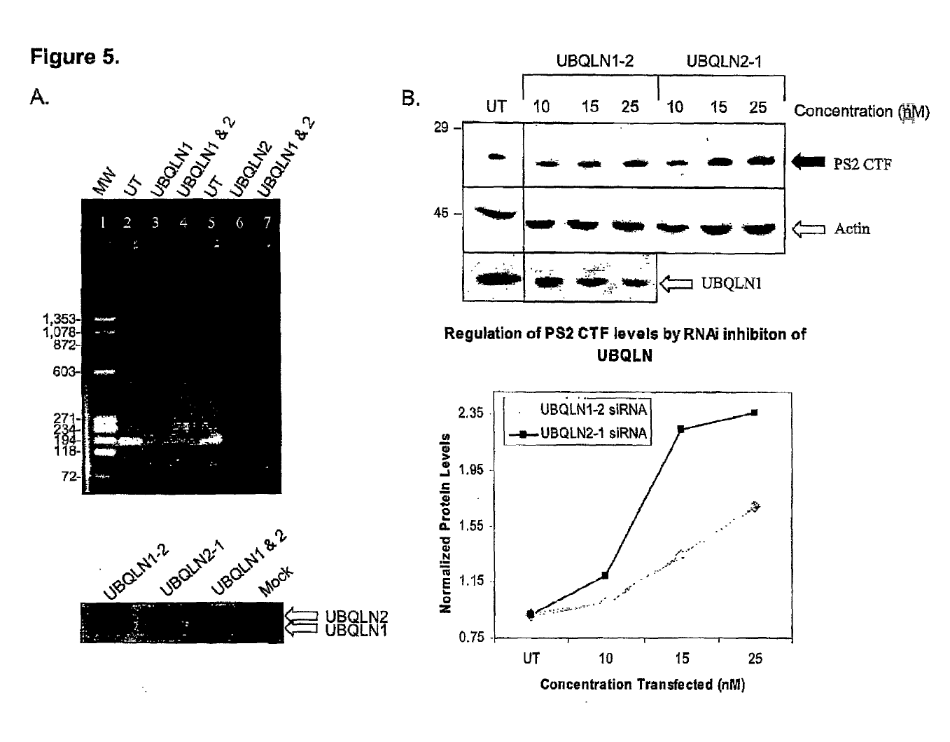

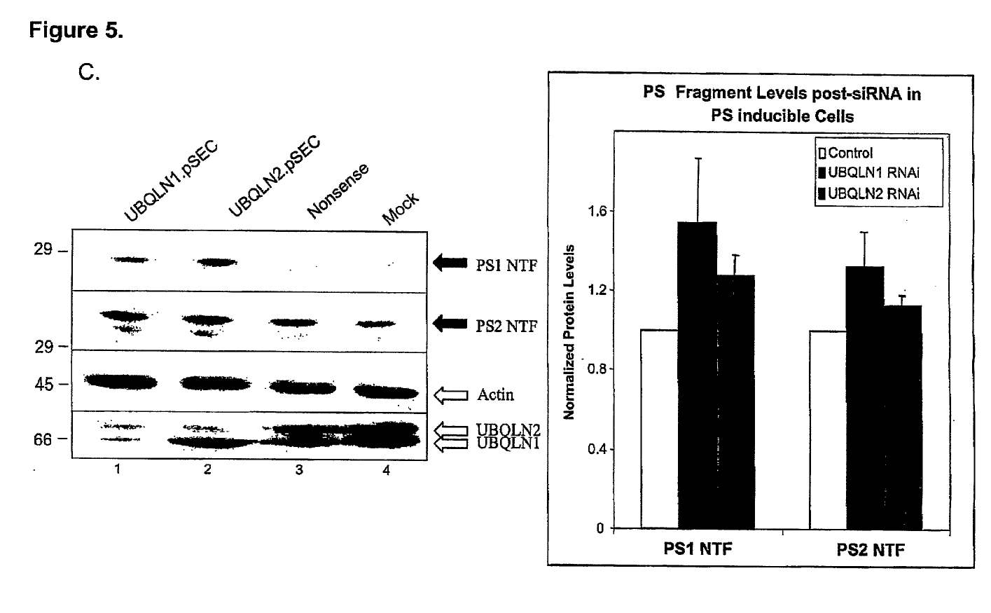

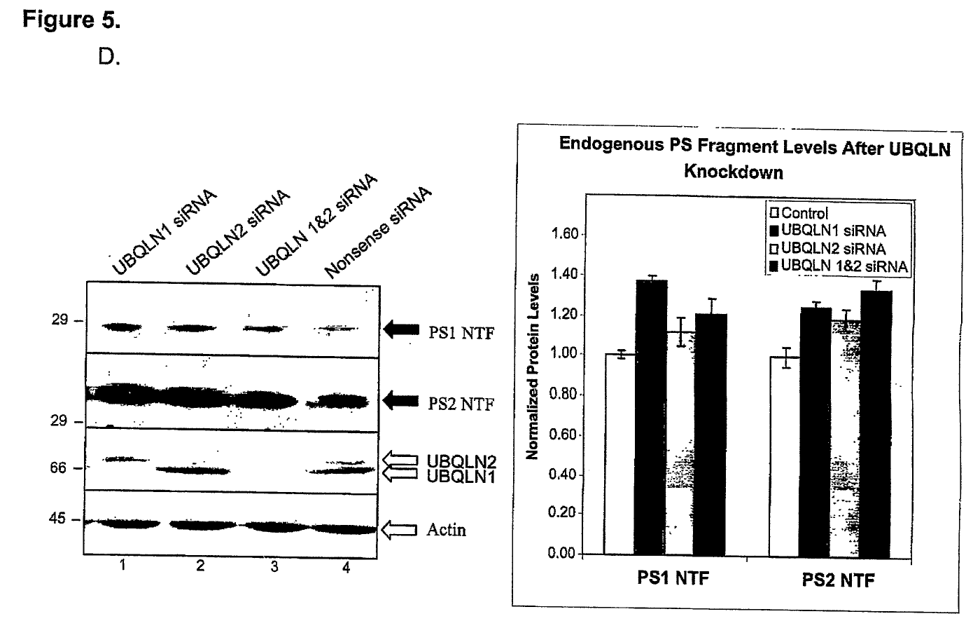

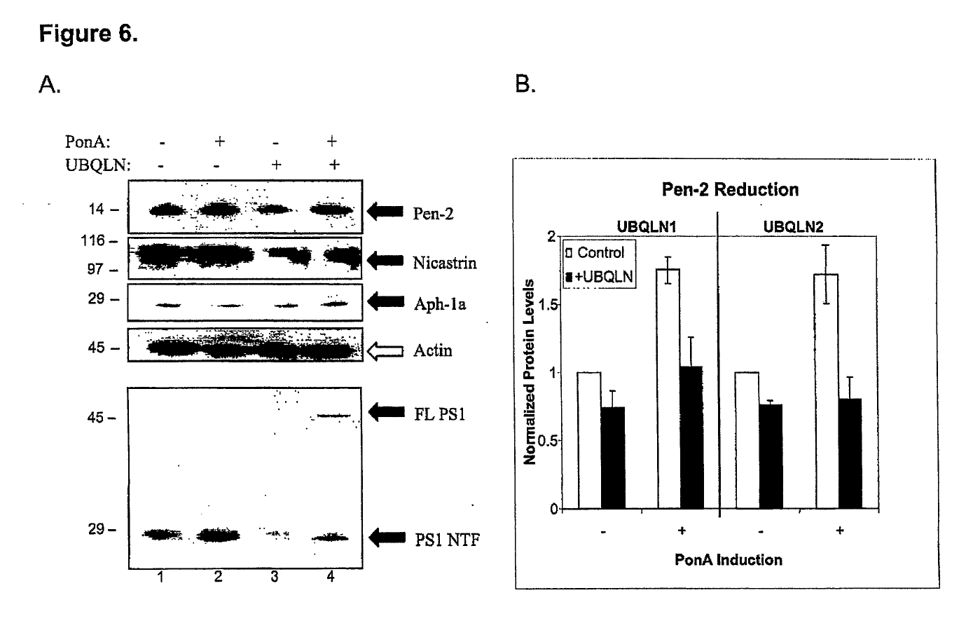

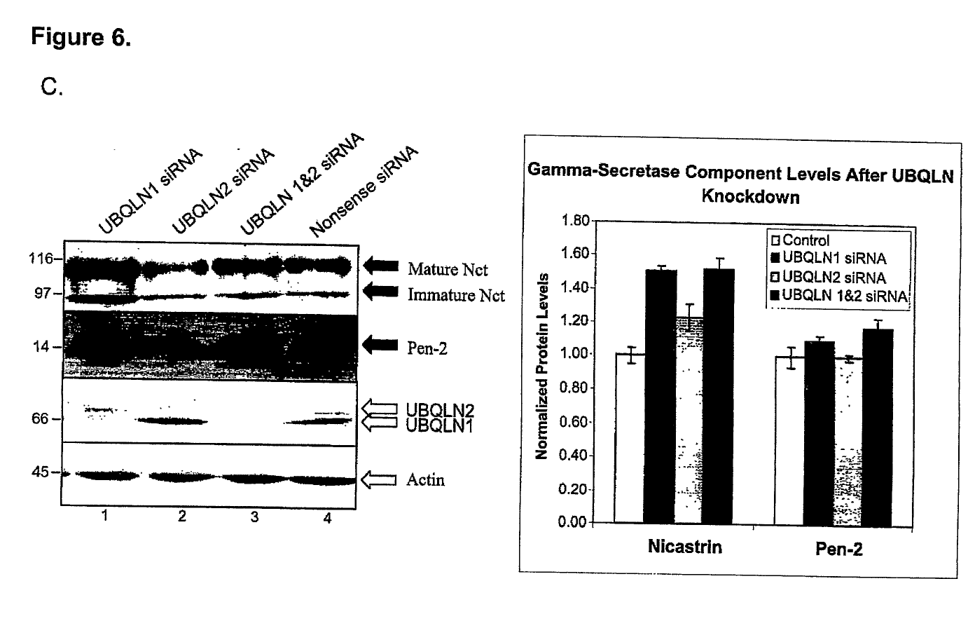

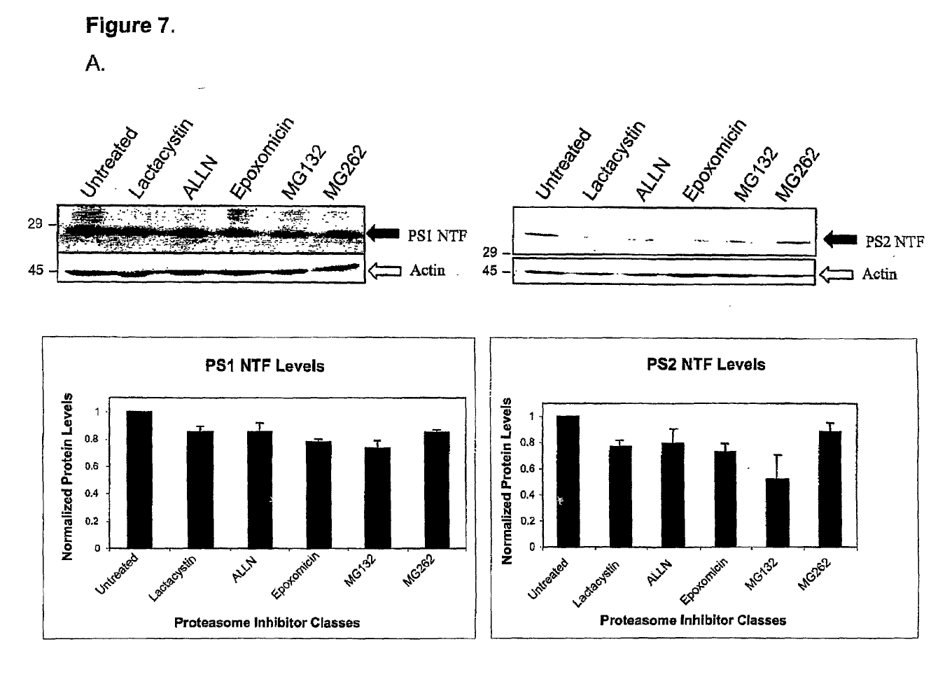

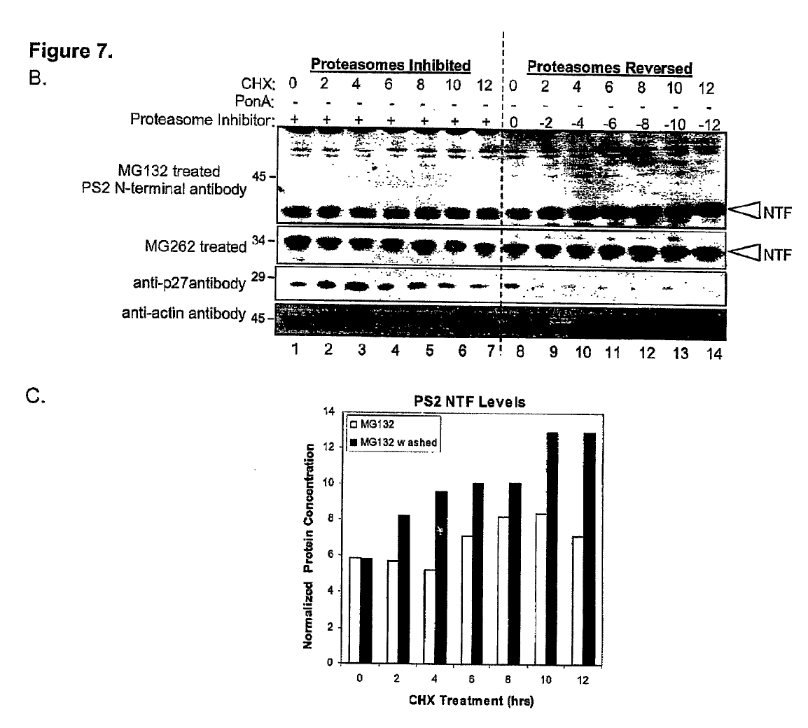

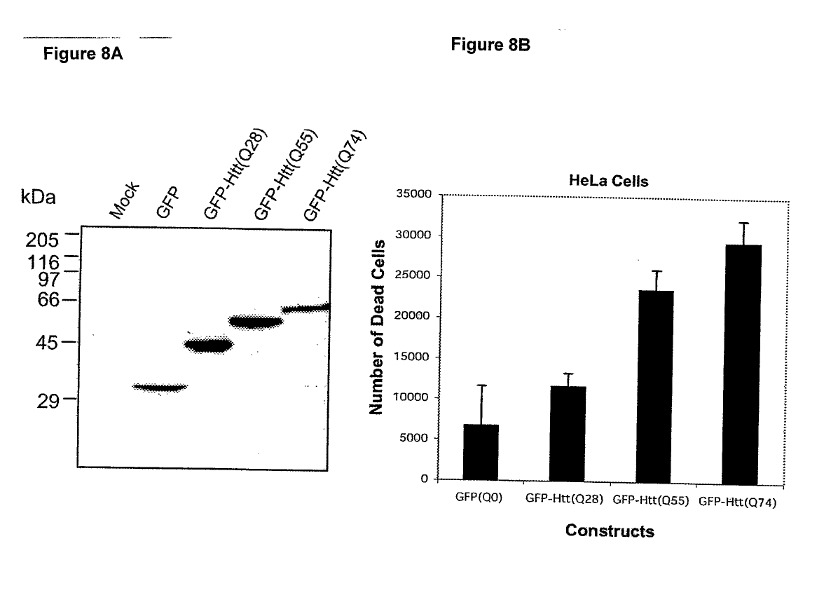

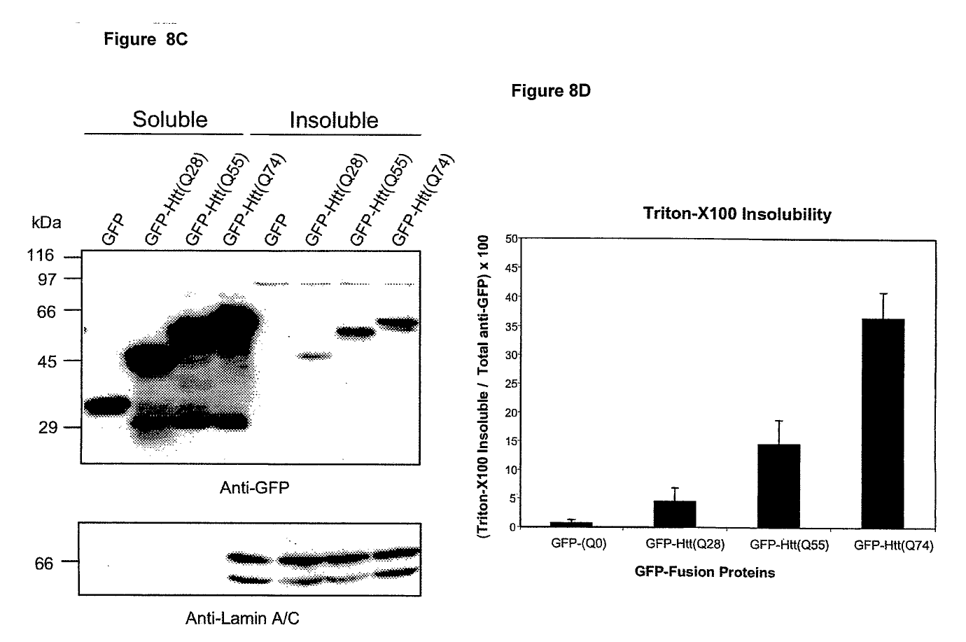

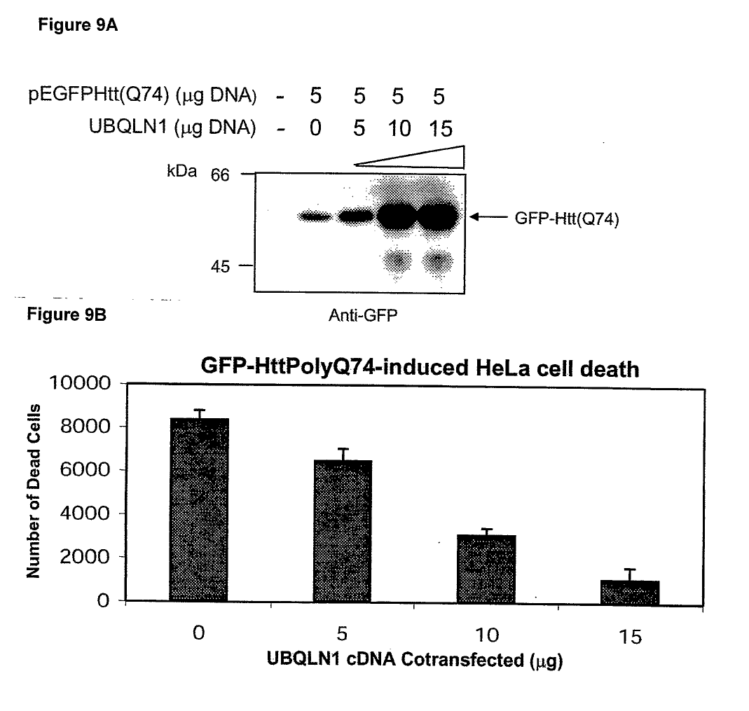

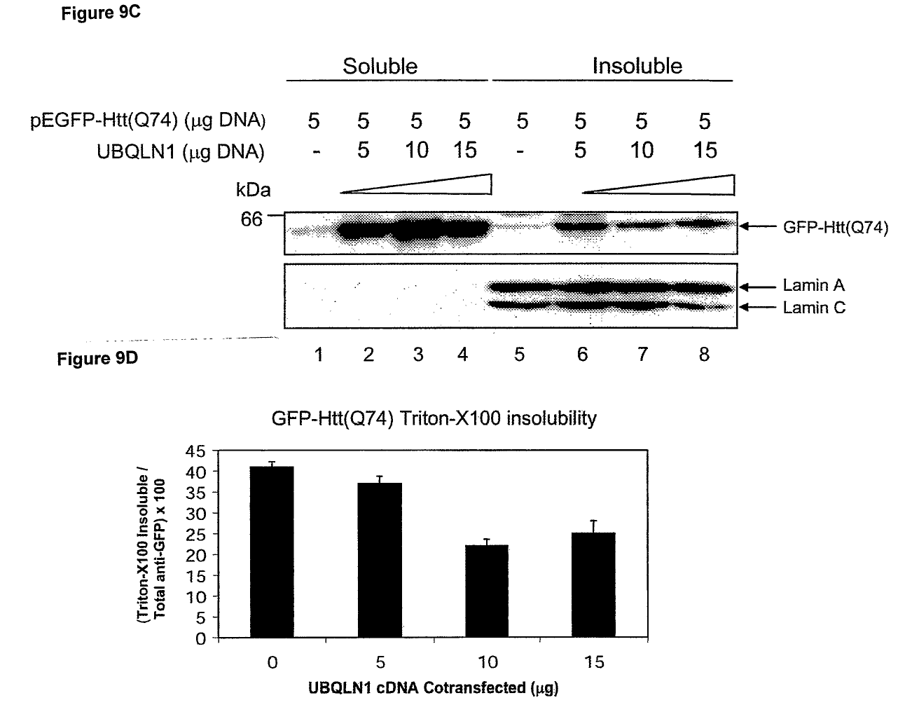

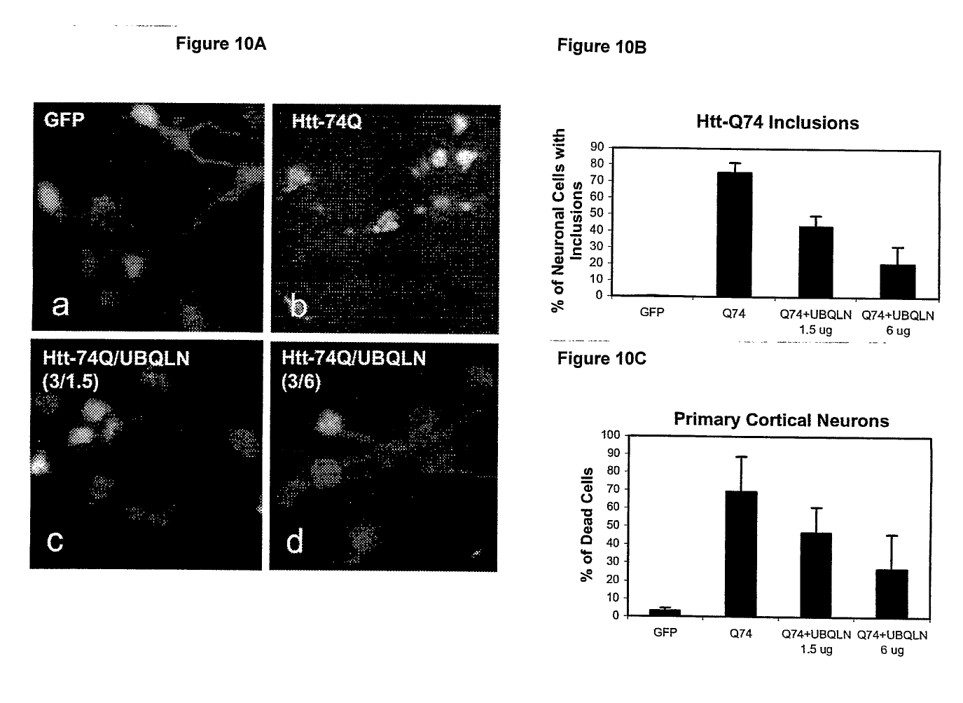

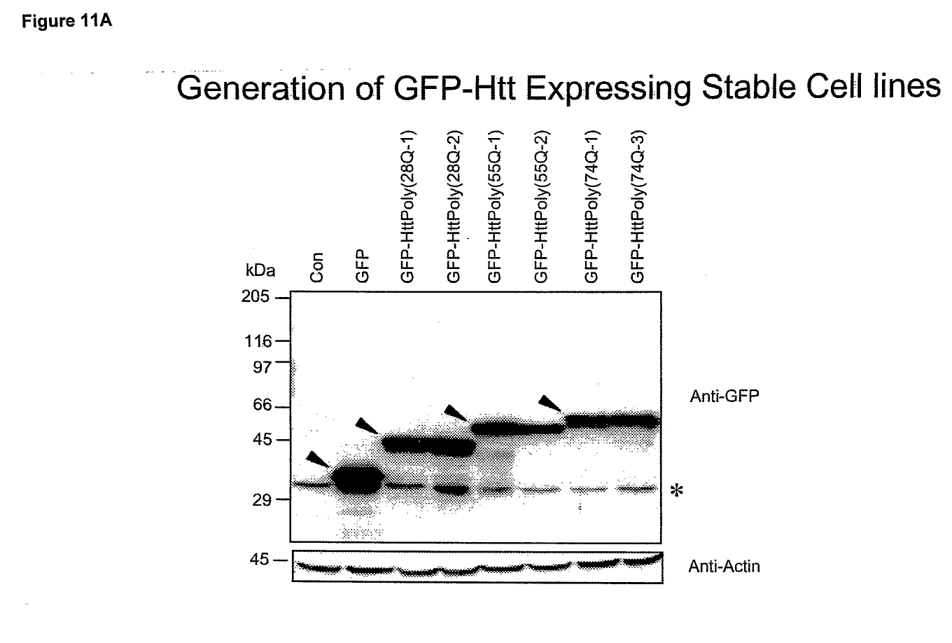

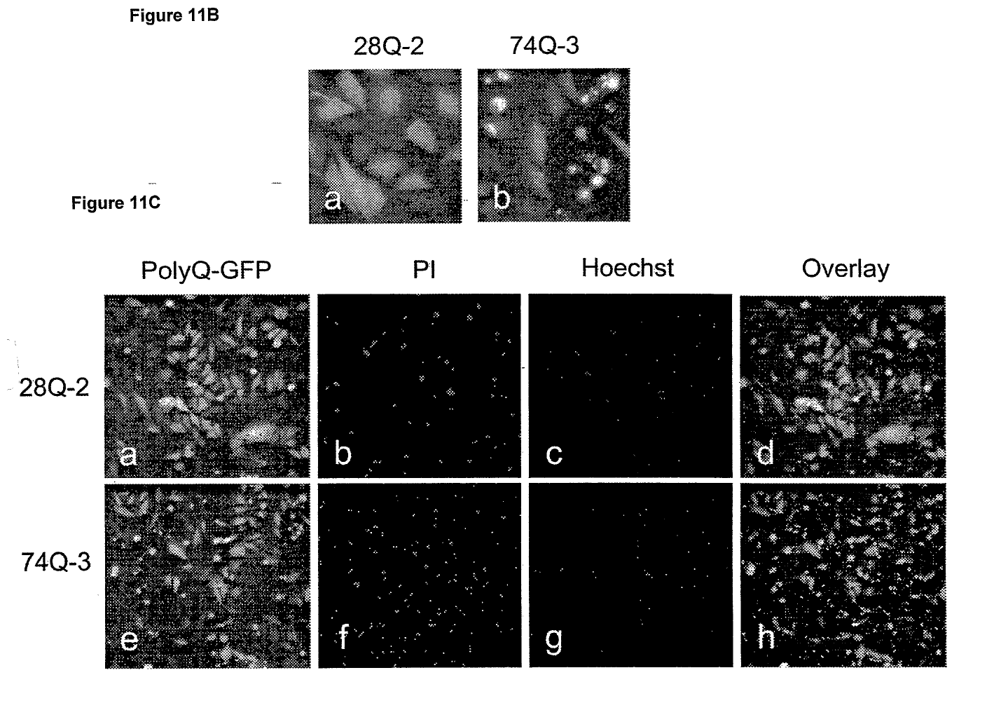

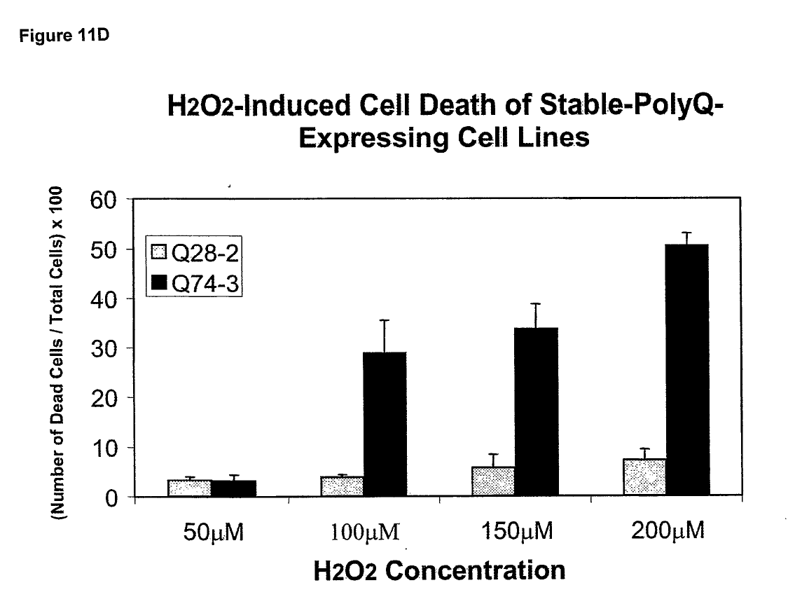

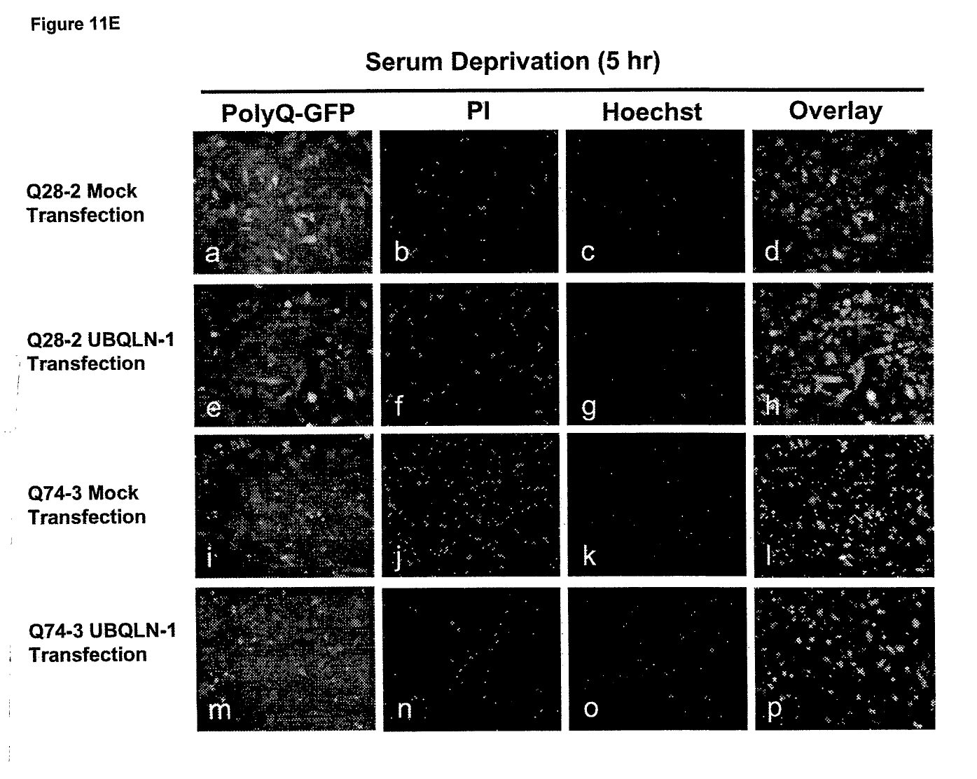

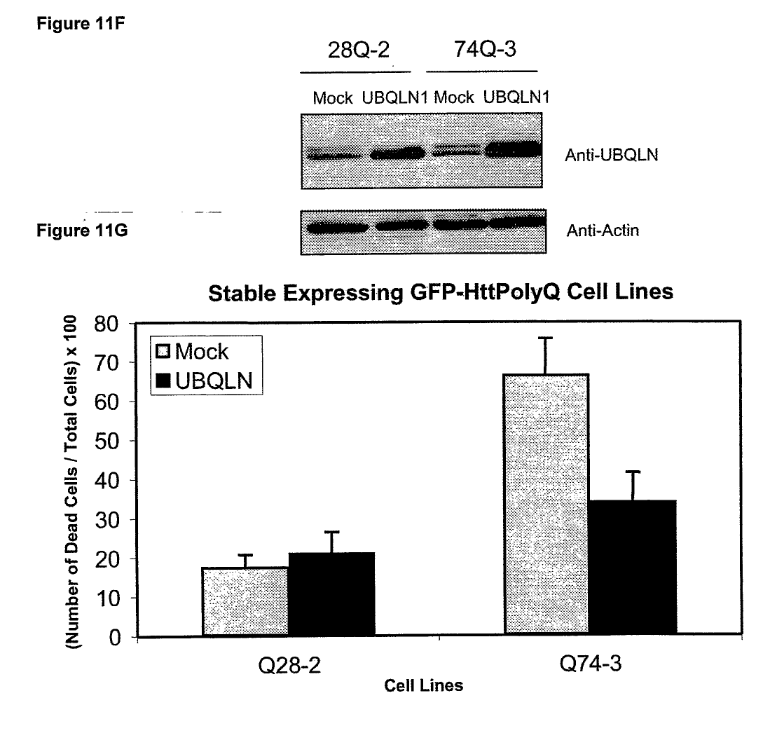

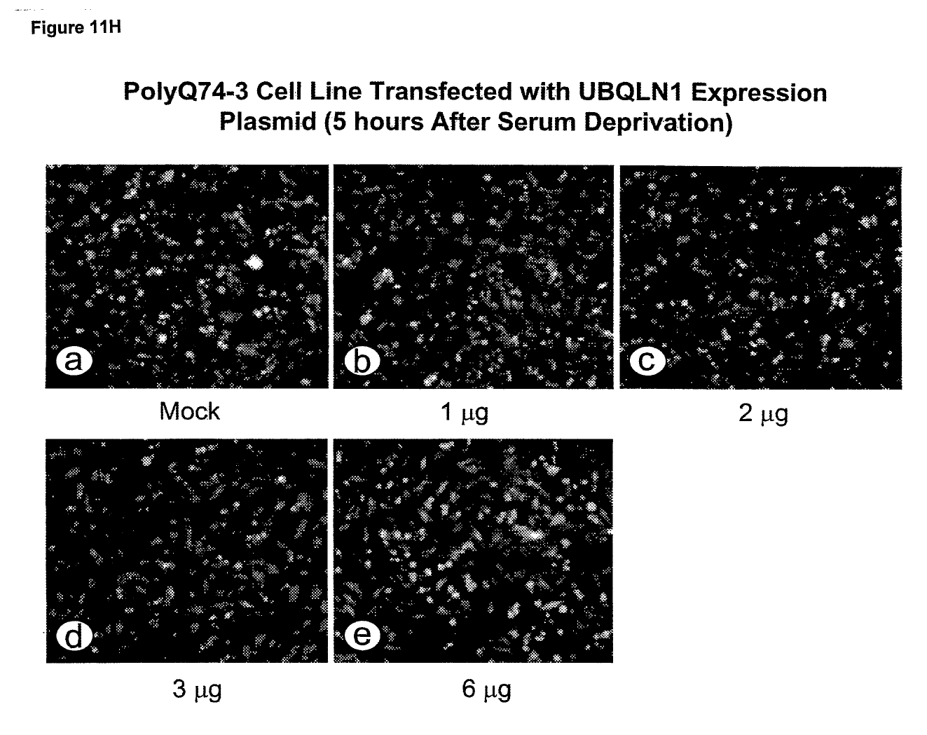

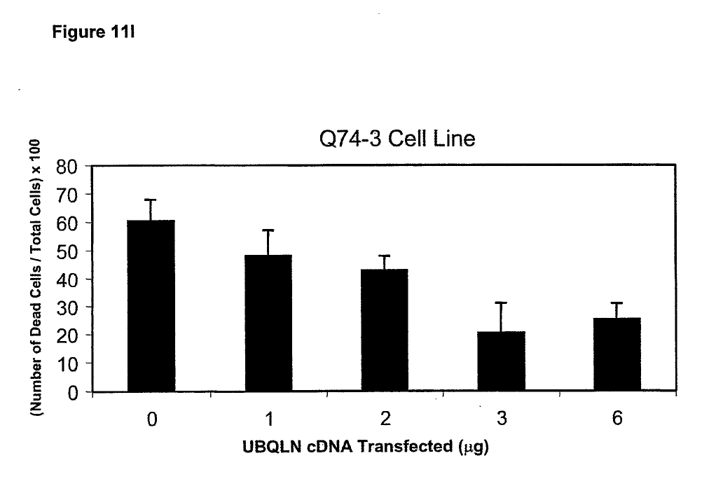

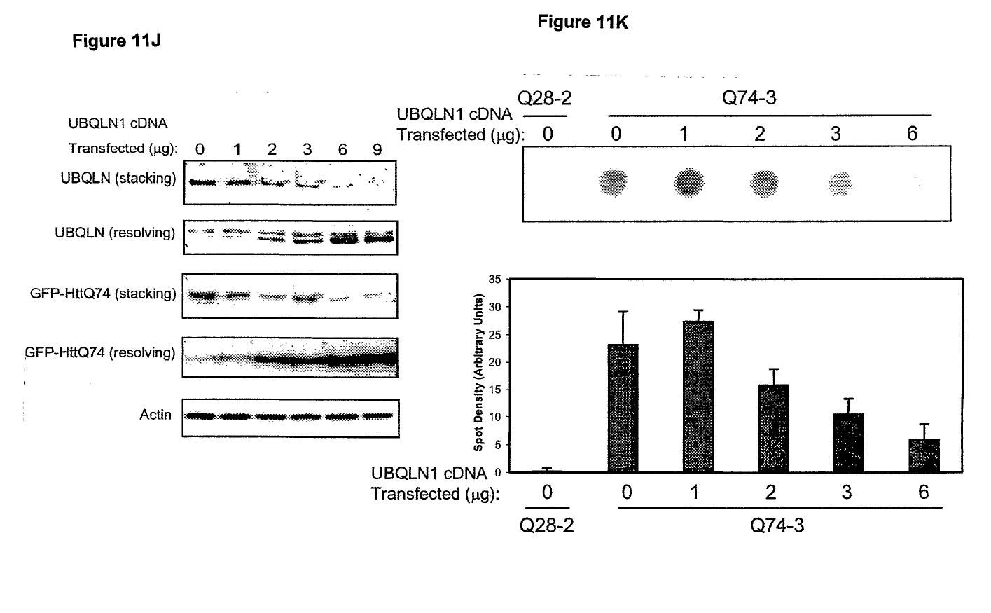

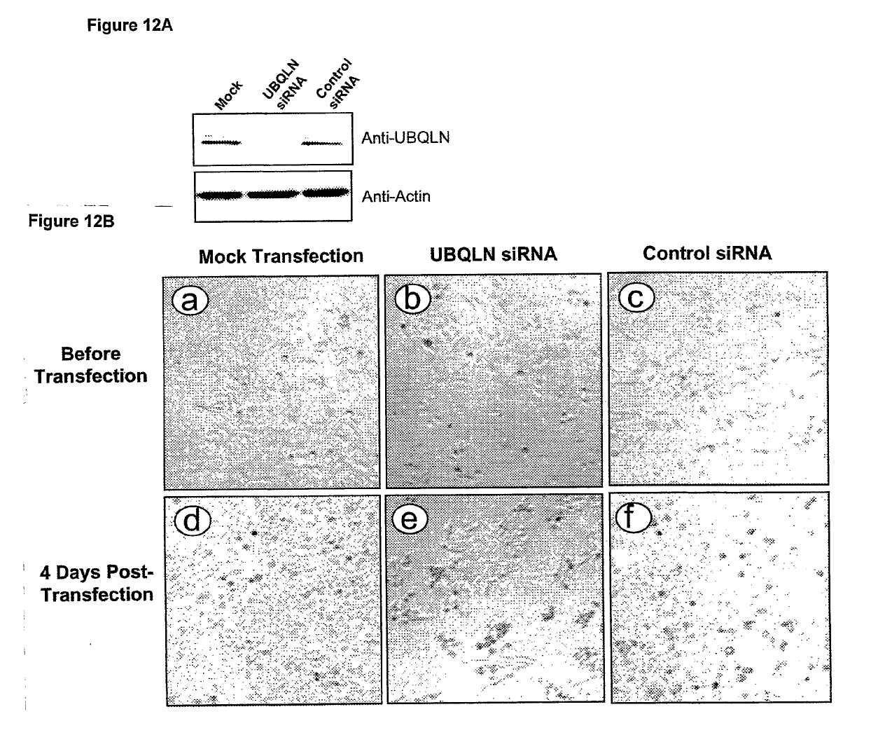

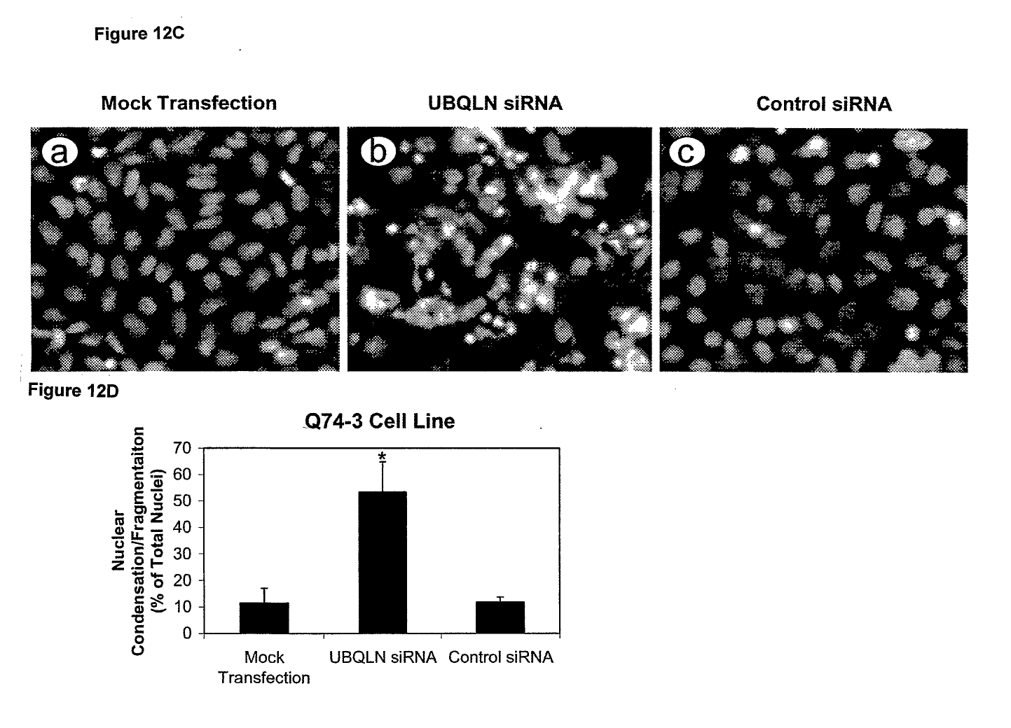

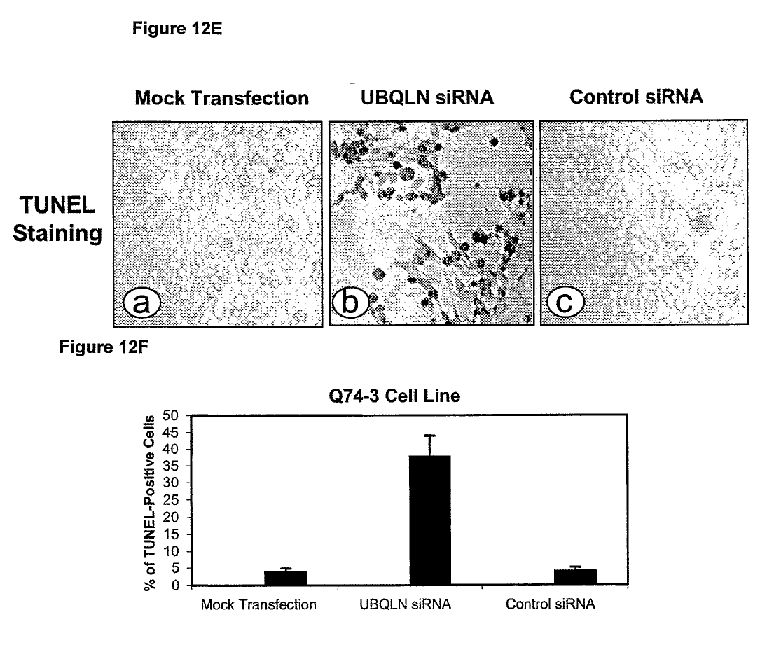

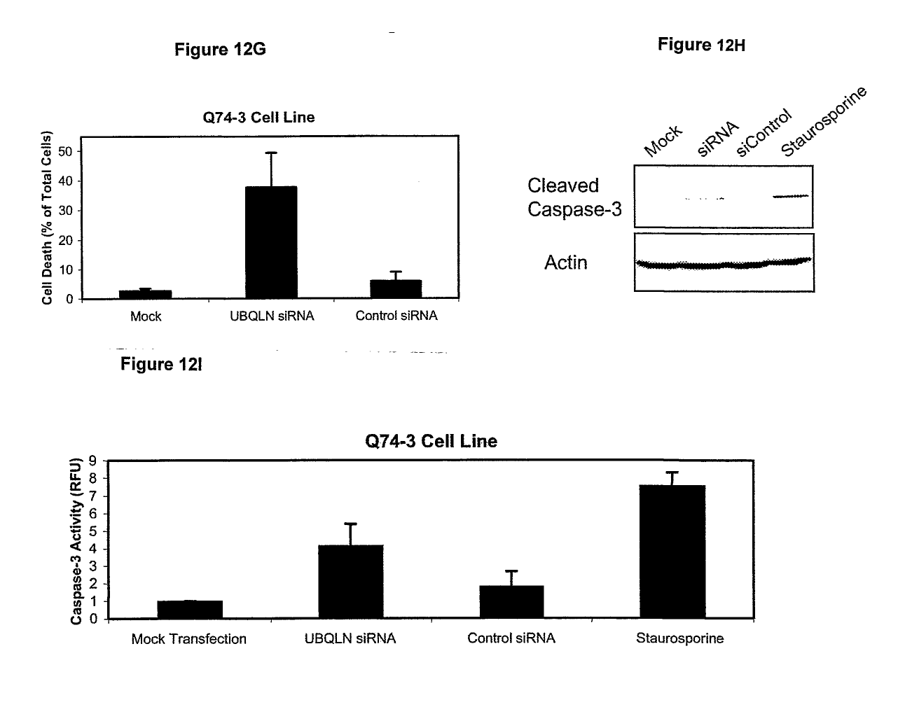

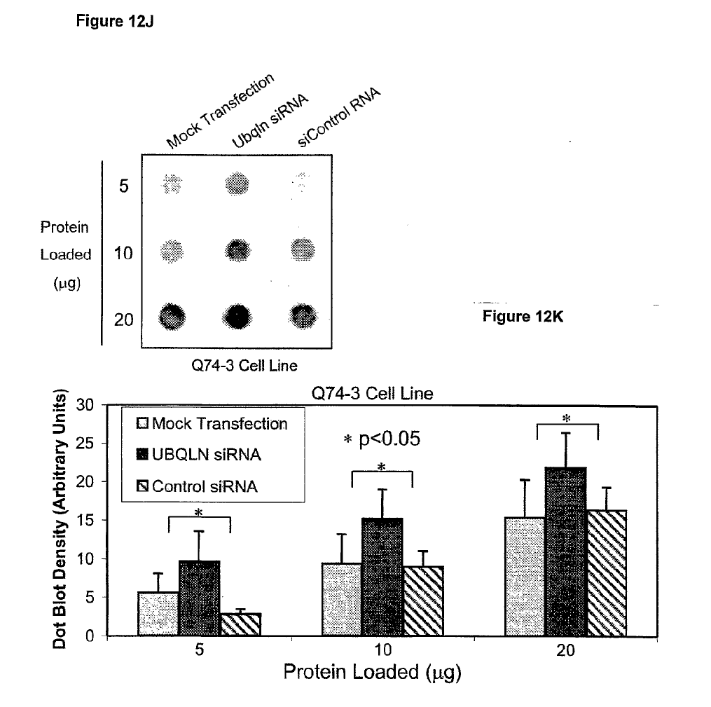

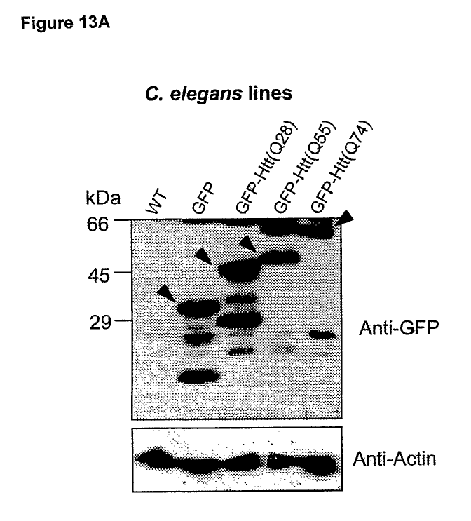

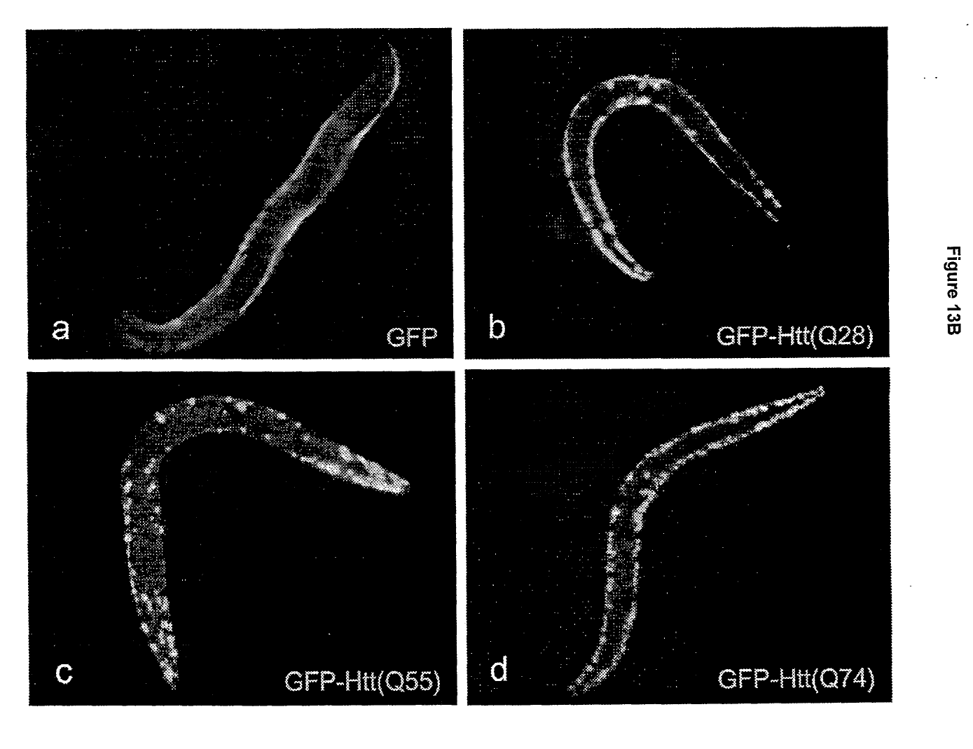

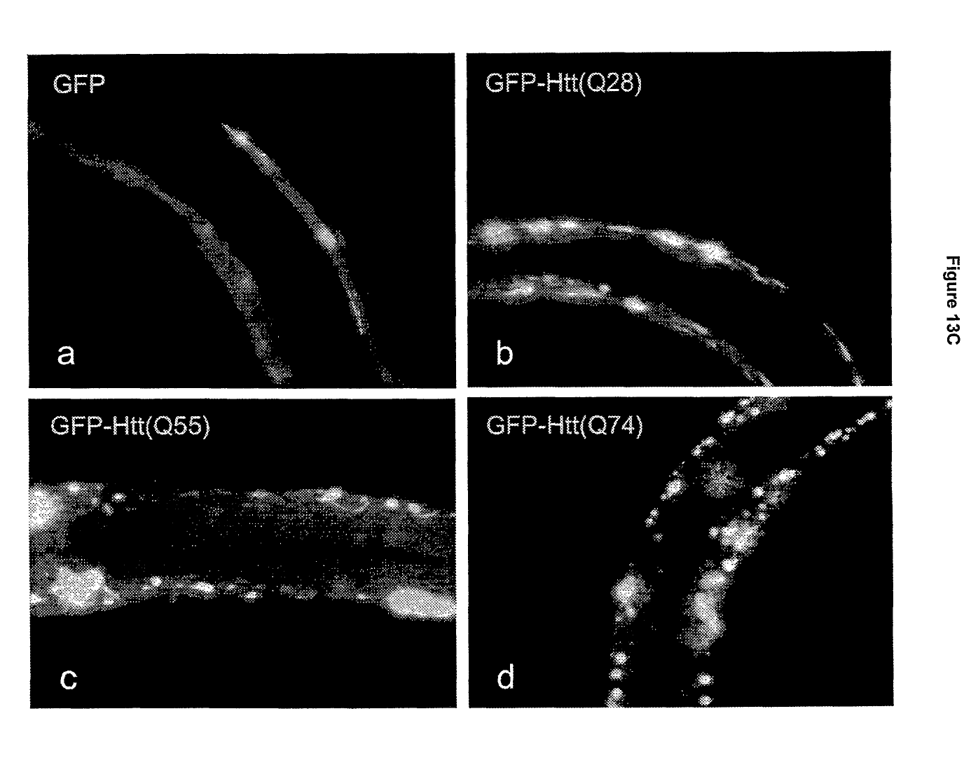

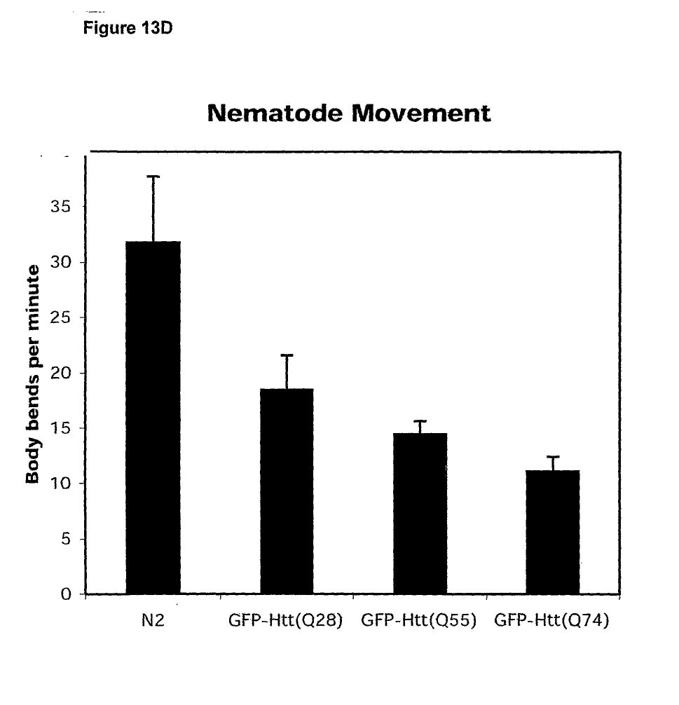

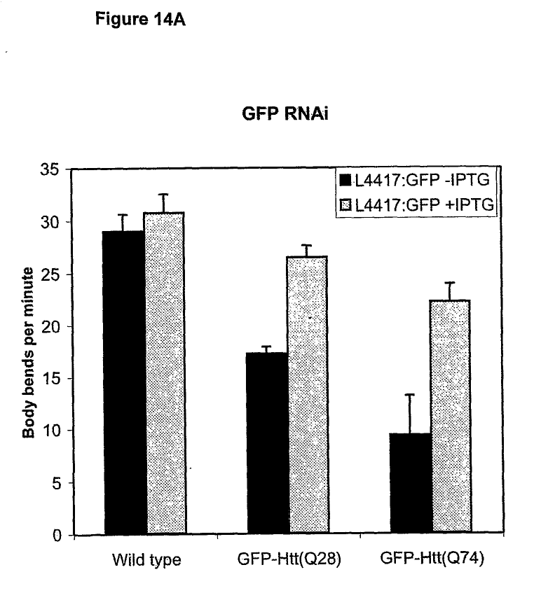

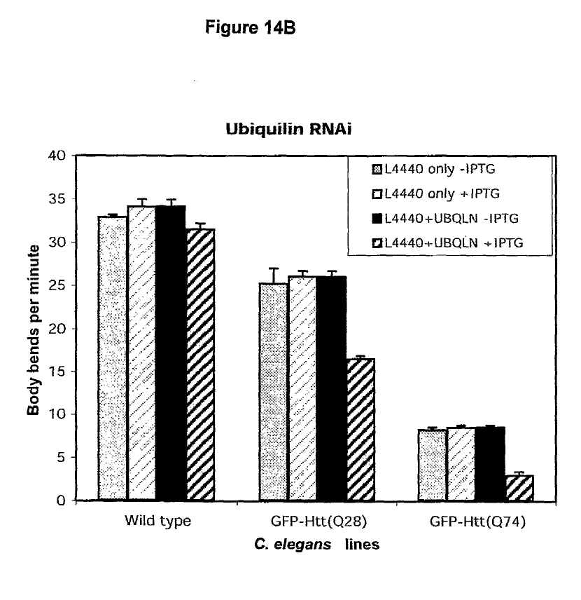

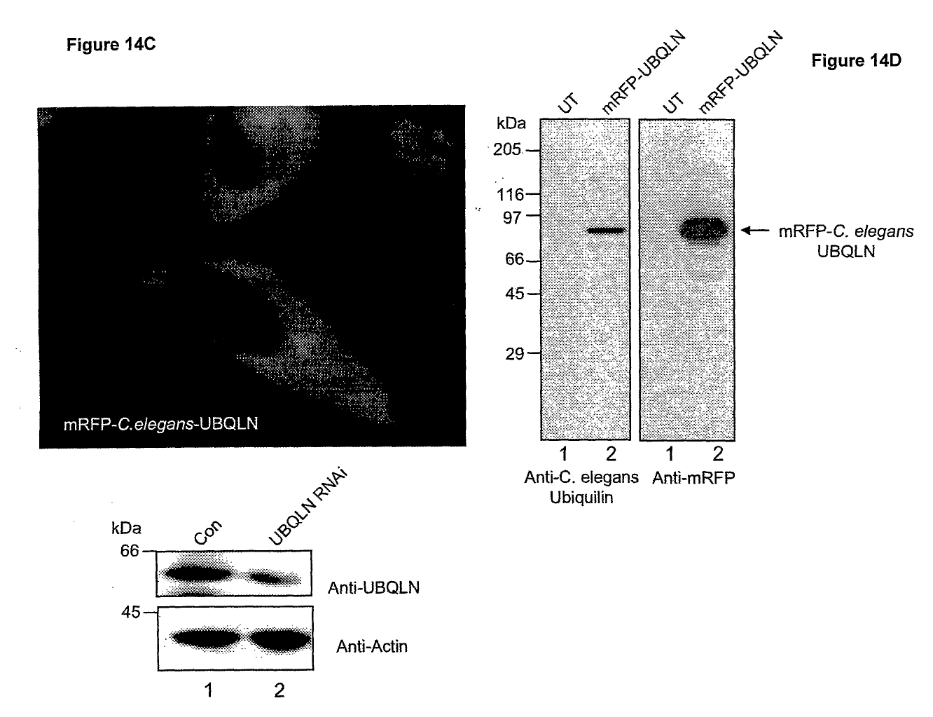

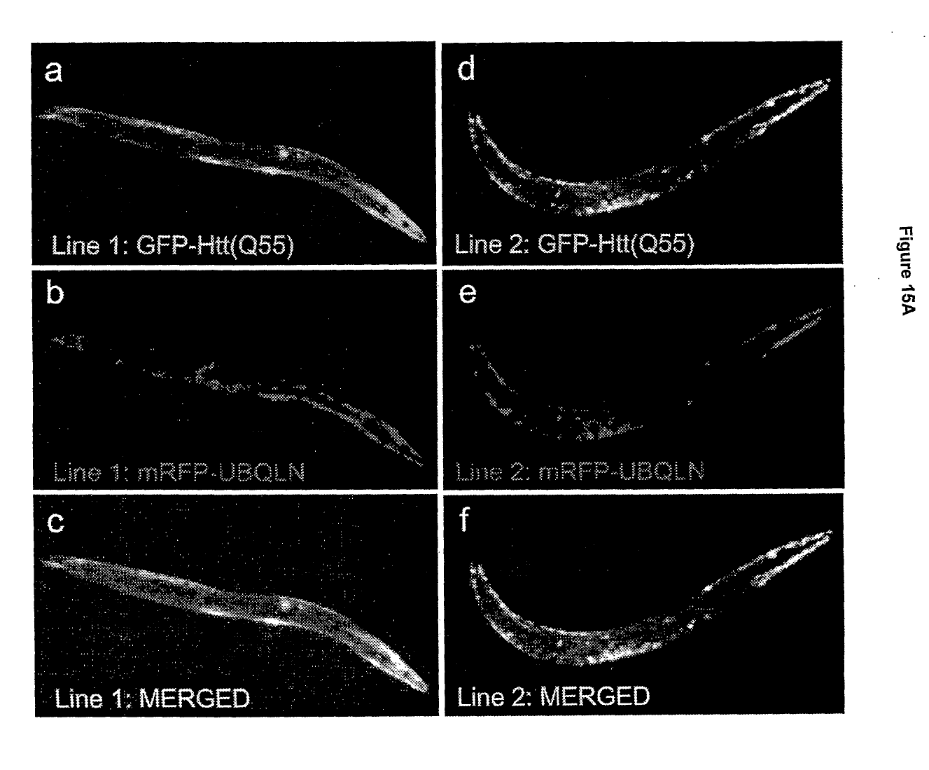

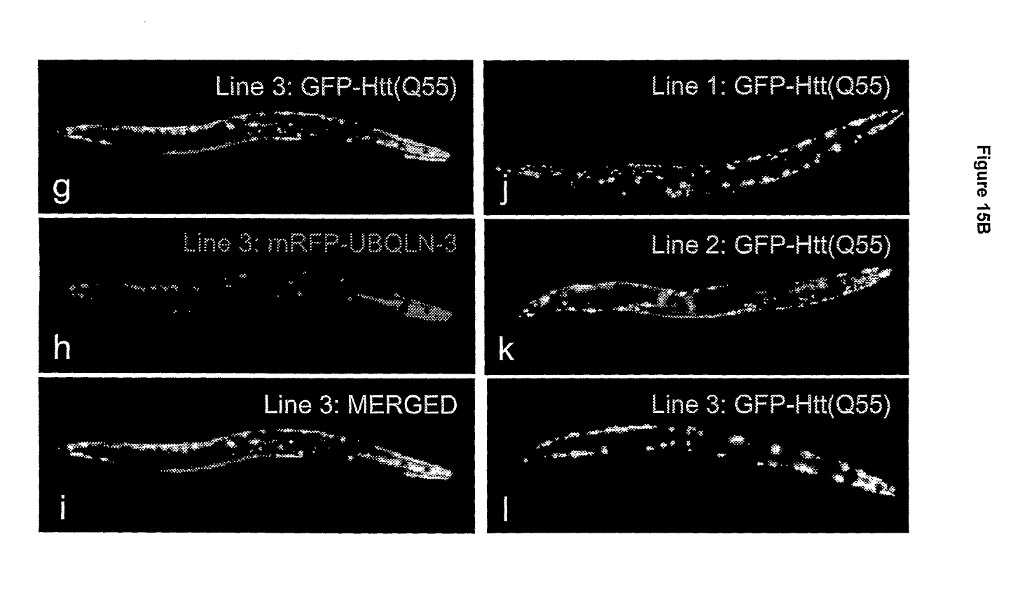

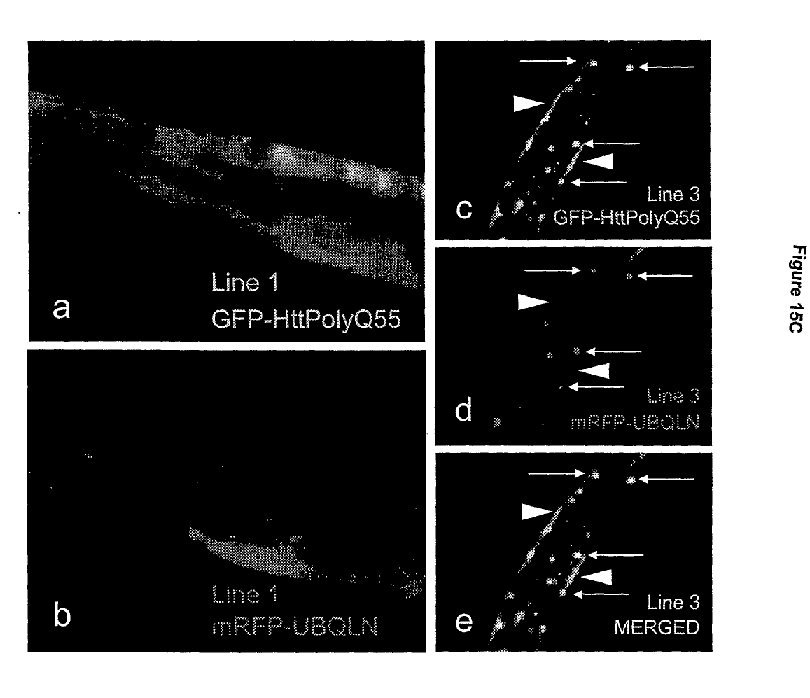

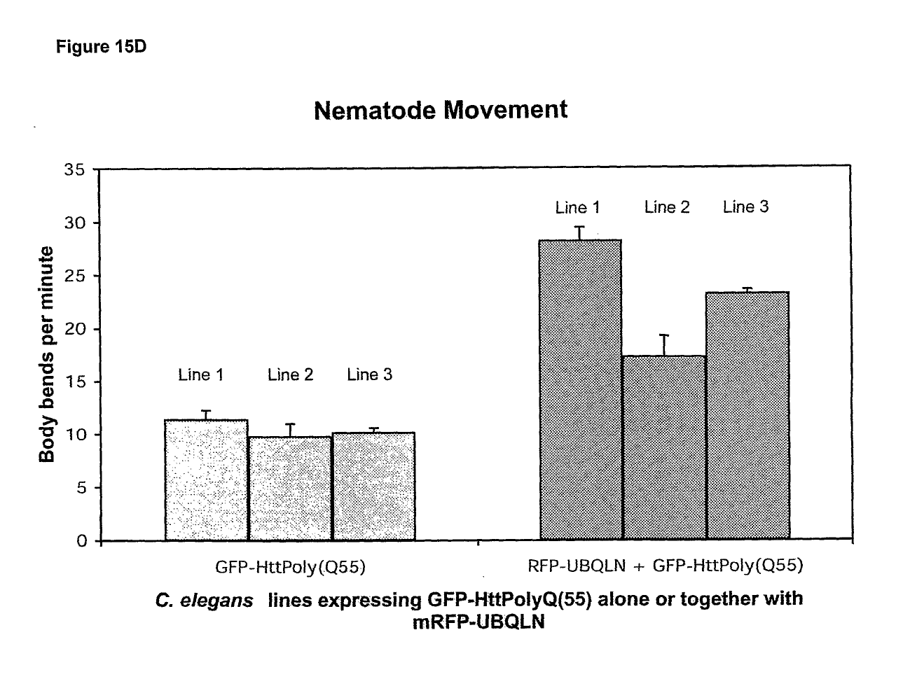

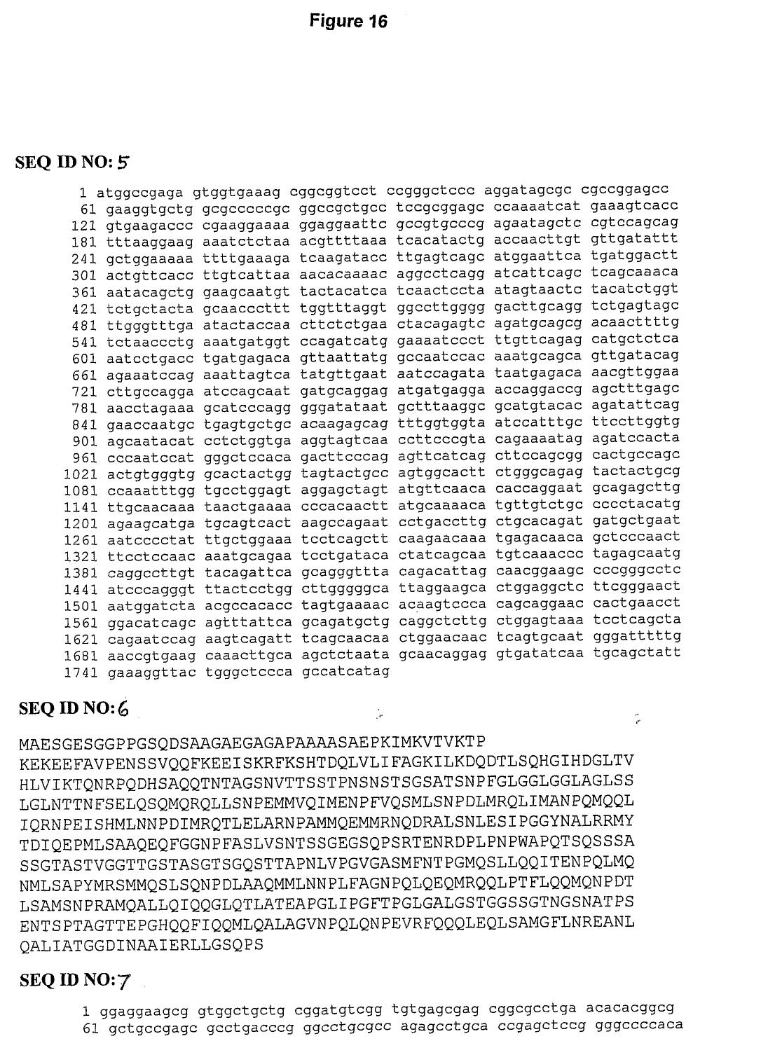

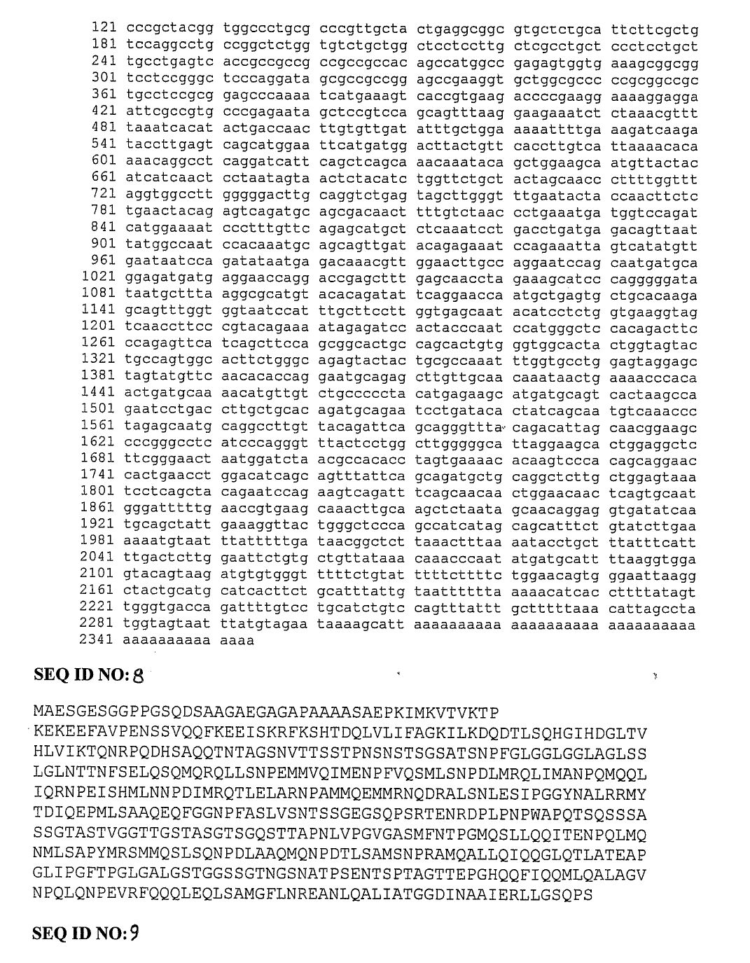

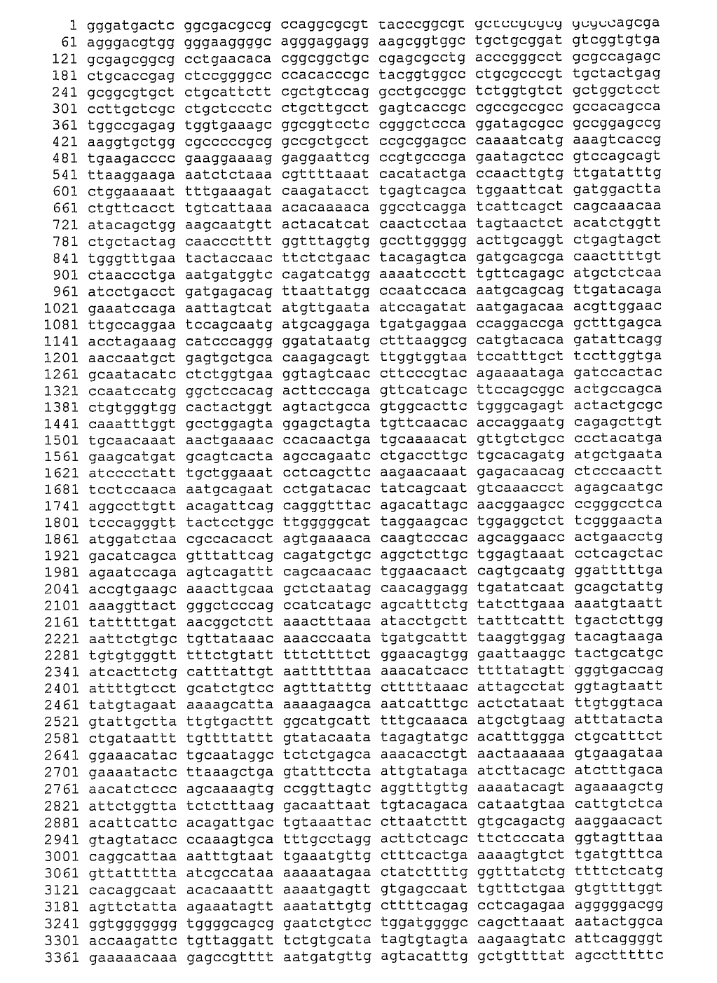

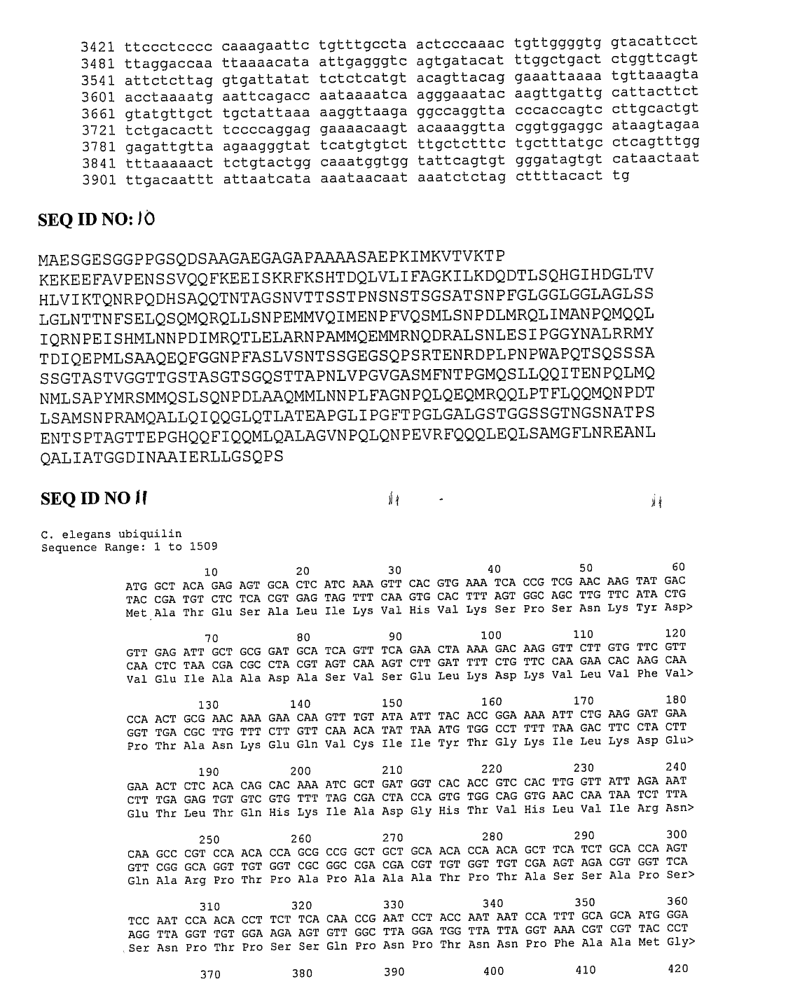

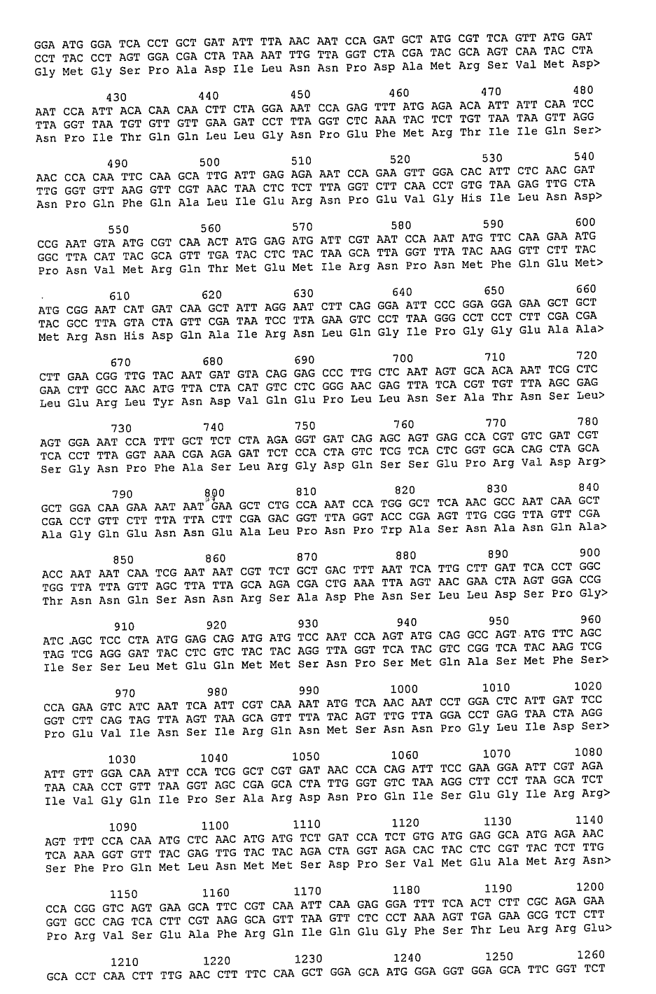

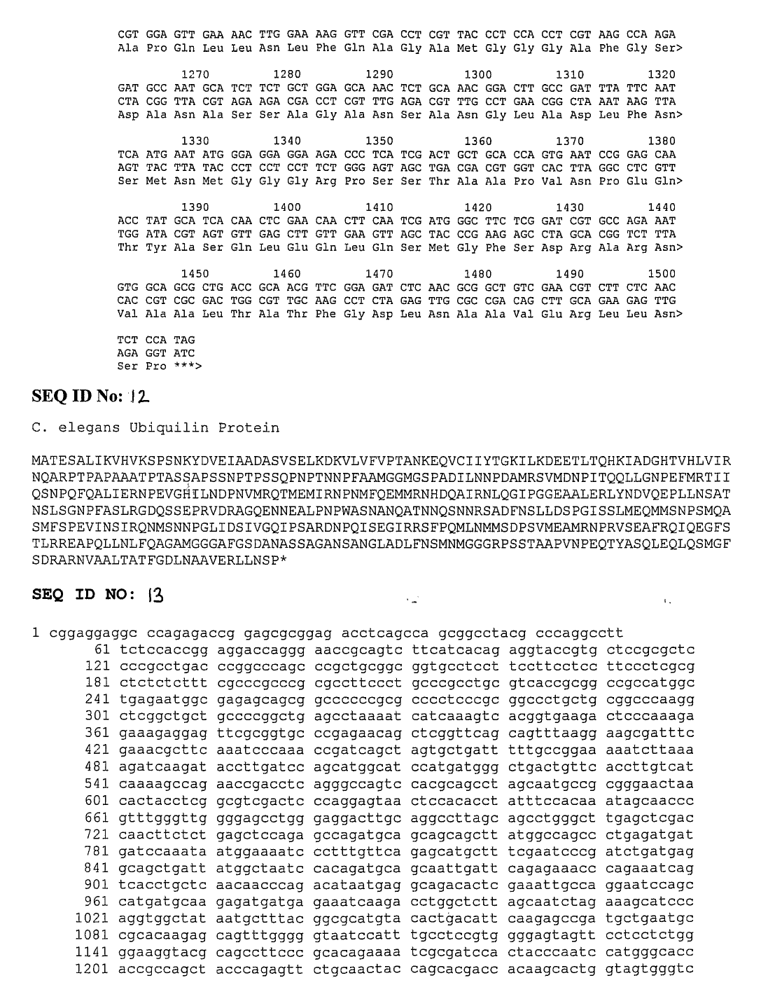

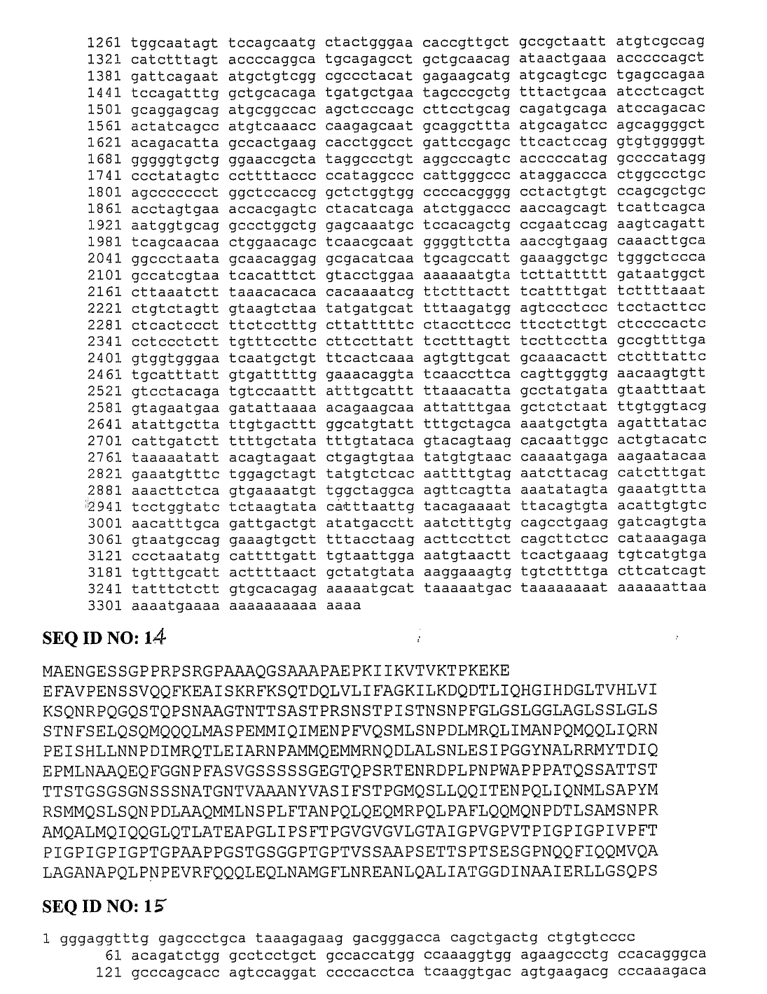

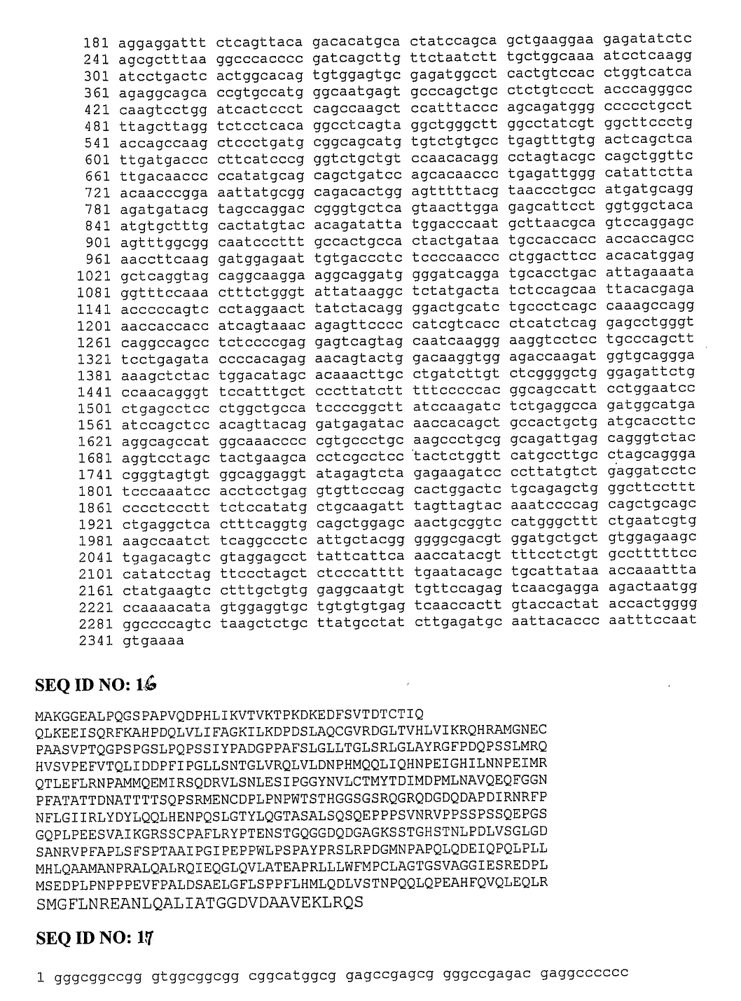

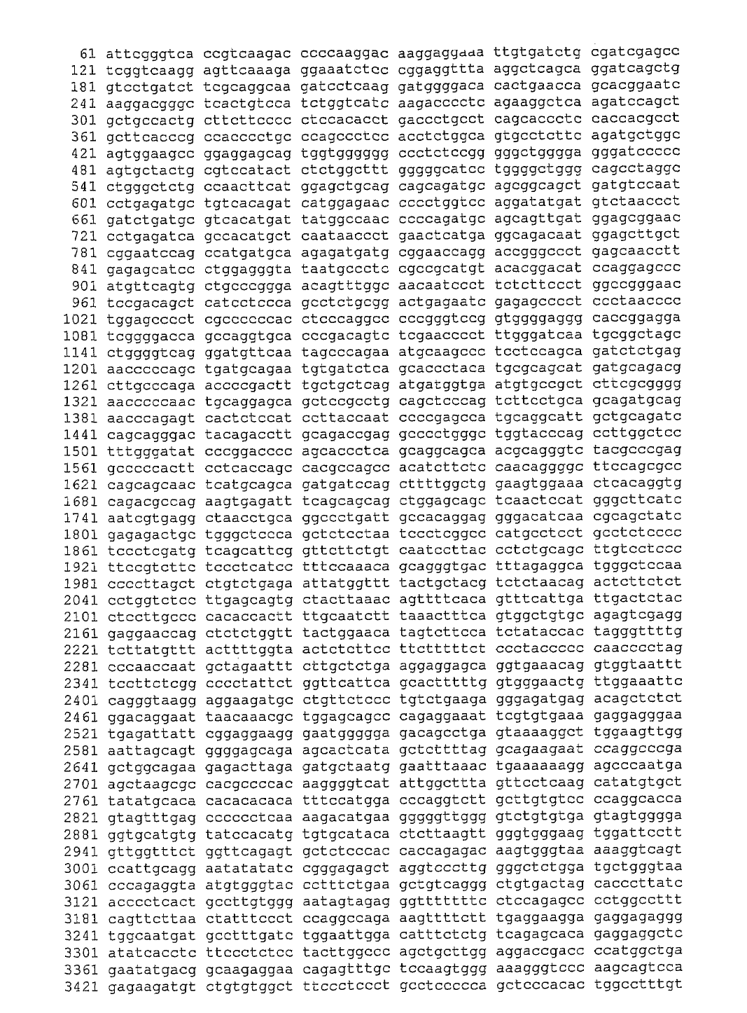

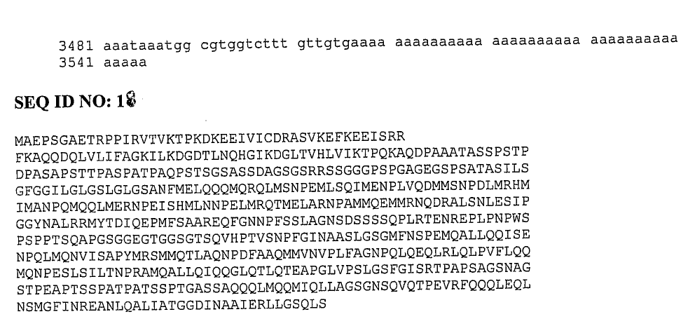

Previous experiments showed that overexpression of UBQLN increased the synthesis of FL PS proteins and decreased the turnover of HMwt PS proteins in cells [43]. However, the effects on PS fragments were not examined. To this end, stable HEK293 cell lines which inducibly express PS1 or PS2 were generated and UBQLN's effect on endoproteolysis was further characterized. Cells were transiently transfected with UBQLN1 cDNA plasmids and then induced for PS expression using PonA. Cell lysates were collected the next day and separated by SDS-PAGE. Surprisingly contrary to its effects on FL and HMwt PS proteins, instead of increasing fragment levels, UBQLN1 overexpression decreased both the NTF and CTF of both PS1 and PS2 suggesting that UBQLN either prevents PS endoproteolysis or enhances the degradation of the PS fragments (FIG. 1A and 1B, compare lanes 1 and 3 or 2 and 4). To confirm the effect was not due to differences in antibody detection, different anti-PS2 antibodies were used that were specific to either the NH,-terminus or the COOH-terminal loop domain of the PS2 proteins, and similar results were observed (FIG. 1B).

Densitometric analysis was used to quantify the extent of reduction which revealed a maximum of a 1.3-fold reduction in PS1 fragments and a 5-fold reduction in PS2 fragments indicating that while UBQLN1 acts on both PS1 and PS2, it exerts a stronger effect on PS2 endoproteolysis, which is consistent with UBQLN1's ability to interact more strongly with PS2 as determined by yeast two-hybrid studies [43]. To confirm that the reduction in PS fragments were not specific to the PS stable cell lines, the effects of UBQLN overexpression on endogenous PS fragments was examined in wild-type HEK293 cells. Wild-type HEK293 cells (i.e. that had not been stably transfected with PS constructs) were transfected with increasing amounts of UBQLN1 expression plasmid and then equal amounts of protein lysate was probed for PS fragments by immunoblot analysis. Similar to the effects seen with the stable PS inducible cells, UBQLN1 overexpression reduced endogenous PS1 NTF levels in normal HEK293 cells by approximately 1.7-fold (FIG. 1C). All of the experiments were reproduced, overexpressing UBQLN2 instead of UBQLN1, and found similar results (data not shown). Because it was considered important to illustrate the effects on endogenous PS fragment levels from overexpression of UBQLN2, results for this experiment is the only one shown (FIG. 1C). Like UBQLN1, increased levels of UBQLN2 reduced endogenous PS1 NTF levels by approximately 1.7-fold

Overexpression of UBQLN Inhibits PS Endoproteolysis

The above results raised an important question: what is the mechanism by which UBQLN overexpression decreases PS fragment levels? Does UBQLN, through its ability to interact with PS polypeptides and proteasomal subunits, escort PS2 fragments to the proteasome to facilitate their rapid degradation (FIG. 2A), or does it prevent production of the PS fragments by inhibiting the so-called “presenilinase” (FIG. 2B). If UBQLN acts to increase the rate of degradation of the PS fragments thereby decreasing their levels, it was expected that by treating cells with a proteasome inhibitor, like MG132 to block proteasomal-dependent degradation, PS fragment levels would be restored to the same level as in untransfected lysates. Furthermore, a classical pulse-chase study would also demonstrate whether or not UBQLN increased the rate of turnover of the PS fragments. On the other hand, if UBQLN overexpression decreases the production of the PS fragments, then when monitoring PS fragment production over time UBQLN overexpressing cells would show lower fragment levels and a slower rate of production when compared with untransfected cells.

To address whether UBQLN was decreasing fragment levels by enhancing their turnover, the PS1 and PS2 stable cell lines were either transfected with UBQLN1 or UBQLN2 expression plasmids or left untransfected, subsequently induced for PS expression with PonA, and the next day treated with the proteasome inhibitor MG 132. Both PS1 and PS2 NTF and CTF levels remained reduced after UBQLN1 transfection and MG 132 treatment (FIG. 3A and B, compare lanes 1 and 3 or 2 and 4). Once again, the extent of reduction was greater in the PS2 stable cells suggesting that UBQLN1 has stronger effects on PS2 endoproteolysis. An interesting trend was observed; namely, cells treated with MG132 had reduced PS fragment levels compared with MG132 untreated cells suggesting that the proteasome is involved in PS endoproteolysis (see below). Again, the experiments were repeated with UBQLN2 and similar trends were observed implying that UBQLN1 and UBQLN2 exert similar effects on PS endoproteolysis.

While these results indicated that overexpression of UBQLN1 does not enhance PS fragment degradation, it was important to show this through a different method. Hence a classical pulse-chase experiment was performed to monitor fragment turnover with or without UBQLN overexpression. There was no significant difference in the turnover of PS2 NTF after a 35 hour chase regardless of whether UBQLN was overexpressed (data not shown). Again, these results confirm that UBQLN does not enhance fragment degradation. It was next necessary to demonstrate that UBQLN actually blocked PS endoproteolysis. To demonstrate this, an experiment similar in concept to a pulse-chase was used, but protein production is monitored instead of turnover. To this end, protein synthesis was inhibited using cyclohexamide (CHX) and PS fragment production was monitored over a six-hour time period in UBQLN transfected and untransfected lysates that were either induced or uninduced for PS expression. UBQLN immunoblots demonstrated that UBLQN1 was indeed overexpressed 4-fold in transfected lysates, and an actin immunoblot confirmed that equal amounts of protein lysates had been loaded (FIG. 4A and B). Interestingly, cells overexpressing UBQLN1 had 1.2 to 1.6-fold lower levels of PS2 NTF than untransfected cells. Densitometric analysis of PS fragment accumulation over time in PS induced and uninduced cells treated with CHX indicated that UBQLN1 overexpression slows the rate of PS2 NTF production (FIG. 4C). Similar results were obtained with UBQLN2 (data not shown). Together, these results are consistent with the idea that UBQLN1 and UBQLN2 inhibit PS endoproteolysis by a similar mechanism.

RNAi-mediated reduction of UBQLN leads to increased PS endoproteolysis

All of the initial studies were based on UBQLN overexpression strategies. The next logical step was to determine if reducing UBQLN levels has the opposite effect of overexpression, leading to an increase in PS fragments. Four small interfering RNAs (siRNAs) were generated, two that were specific for UBQLN1, termed UBQLN1-1 and UBQLN1-2, and two that were specific for UBQLN2, termed UBQLN2-1 and UBQLN2-2. The siRNAs were transfected in increasing concentrations into HeLa cells and cell lysates were collected after 48 hours, separated by SDS-PAGE, and levels of different proteins analyzed by immunoblotting. Two of the four UBQLN siRNAs, UBQLN1-2 and UBQLN2-1 were successful at reducing UBQLN protein levels as determined by immunoblot analysis (FIG. 5A and data not shown). UBQLN1 protein levels were reduced by 72% while UBQLN2 protein levels were only reduced by 46%. However, to ensure that the UBQLN siRNAs were functioning through the classical RNAi mechanism, RT-PCR experiments were conducted using the UBLQN1-2 and UBQLN2-1 siRNAs, indicating that the UBQLN message was also significantly reduced in cell lysates transfected with either of the two siRNAs (FIG. 5A). Note, FIGS. 5A and 5B are results from siRNA transfection while FIG. 5C is the results from transfection with RNAi plasmids, and FIG. 5D is the results of transfection with SMARTpools of UBQLN1 or UBQLN2 siRNAs (see Methods), all methods of knockdown were rigorously tested, but knockdown by the RNAi plasmids allows for longer knockdown of message levels while SMARTpool transfection allows for stronger reduction in message levels.

Next it was examined whether reduction in UBQLN protein levels leads to changes in PS fragment levels. PS fragment levels in the PS2 stable cell line grown in the absence of PonA was initially examined. Both PS2 NTF and CTF levels were increased approximately 1.5 fold upon UBQLN1 knock-down consistent with the idea that a reduction of UBQLN levels should cause an increase in PS endoproteolysis (FIG. 5B and C). UBQLN2 knock-down increased PS2 fragments also, but the effect was more variable with a 2.6-fold increase for the CTF and a 1.2- fold increase for the NTF (FIG. 5B and C). UBQLN reduction resulted in an increase in PS1 NTF levels; a 1.6-fold increase was observed with UBQLN1 siRNA transfection and a 1.3-fold increase was seen with UBQLN2 RNAi plasmid transfection.

Next wild-type HEK293 cells were examined to determine if changes in UBQLN levels would affect endogenous PS fragment levels. This time, cells were transfected with UBQLN1 and UBQLN2 SMARTpools, which were very effective at reducing UBQLN protein levels by more than 90% (FIG. 5D). Immunoblot analysis of PS proteins with an anti-PS2 antibody indicated that the PS2 NTF level was increased approximately 1.3 fold upon UBQLN1 knock-down (FIG. 5D). UBQLN2 knock-down increased PS2 fragments also, but not as much as UBQLN1 (FIG. 5D). This is consistent with a smaller reduction in total UBQLN protein levels that can be achieved after UBQLN2 knockdown, because UBQLN1 protein is expressed almost five-fold higher than UBQLN2 in HEK293 cells. Interestingly, when both UBQLN1 and UBQLN2 levels were reduced, the effect was even stronger on the PS2 NTF, with a 1.4-fold increase observed. UBQLN reduction also led to increases in PS1 NTF levels; a 1.4-fold increase was observed with UBQLN1 siRNA transfection and an approximately 1.2-fold increase was seen with UBQLN2 SMARTpool transfection (FIG. 5C). For some unknown reason knockdown of both UBQLN1 and UBQLN2 resulted in an increase in PS 1 NTF that was intermediate to the levels seen upon knockdown of UBQLN1 or UBQLN2 alone, unlike the additive effect that was seen with PS2 NTF. The increases in PS fragment levels upon UBQLN knockdown were very reproducible: similar trends were observed in each of six independent experiments. While these increases appear relatively small, they are likely to be important because levels of endogenous PS fragments are typically invariant and very tightly regulated [34, 35].

Reports have suggested that when PS1 fragment levels increase, there is a concomitant decrease in PS2 fragment levels and vice versa [24, 25]; however, there is debate whether “replacement” is a general phenomenon as it is not always observed [42]. As such, tests were conducted to examined whether PS1 fragment levels were altered in the same siRNA transfected lysates in which an increase in PS2 fragment levels had been observed. Immunoblot analysis indicated that, in fact, both PS1 and PS2 NTF levels increase upon UBQLN protein reduction suggesting that UBQLN acts by a common mechanism on both PS proteins and this effect is upstream of the effecters that are responsible for the replacement effect (FIG. 5C and D).

UBQLN influences -secretase component levels

Since UBQLN overexpression decreases PS fragment levels, the active form of the PS protein which enters the γ-secretase complex, it was next necessary to determine if this change has any effect on the other three known γ-secretase components, Aph-1, Nicastrin, and Pen-2. To this end PS inducible cells were transiently transfected with UBQLN cDNA plasmids and then induced for FL PS expression using PonA. Cell lysates were collected the next day and separated by SDS-PAGE. Immunoblotting with antibodies against the γ-secretase components were used to monitor levels (FIG. 6). Aph-1 levels have been demonstrated to be the least affected by either overexpression or knockdown of the other y-secretase components [3]. As expected, Aph-1 levels remain steady in this experimental system (FIG. 6 A). However, levels of mature Nicastrin decreased when UBQLN was overexpressed (FIG. 6A), which is consistent with previous observations that loss of PS expression correlates with decreased Nicastrin expression [50]. Similarly, upon UBQLN overexpression Pen-2 levels fluctuated in a manner that was directly related to the level of expression of PS fragments in the cell lysates. Thus, when PS1 expression was induced, Pen-2 levels increased by approximately 1.6-fold; yet when UBQLN was overexpressed, Pen-2 levels were reduced by 1.3-fold in the uninduced cell lysates and 2-fold in the induced lysates (FIG. 6A and B). This suggests that UBQLN's ability to decrease the active form of the PS protein causes both Nicastrin and Pen-2 destabilization. Therefore, overexpression of UBQLN reduces the levels of three of the essential y-secretase components) PS fragments, Pen-2 and Nicastrin.

After observing that UBQLN reduction led to an increase in PS fragments, the next step was to determine whether or not this translated to a downstream effect on the γ-secretase components. Indeed, when UBQLN levels were knocked-down in wild-type HEK293 cells there was a 1.5-fold increase in Nicastrin levels (FIG. 6C). This suggests that increased levels of PS fragments leads to stabilization of Nicastrin thereby increasing its levels. There was a smaller effect of UBQLN knockdown on Pen-2 levels with an approximately 1.2-fold increase when both UBQLN1 and UBQLN2 protein levels were reduced by siRNA transfection (FIG. 6C). While the increase is relatively small it was reproduced in six independent experiments lending further support to the hypothesis that UBQLN's modulation of PS fragment levels has downstream effects on the entire γ-secretase complex.

The proteasome is involved in PS2 endoproteolysis

When performing the overexpression studies an interesting an observation was made. Namely, when the PS2 stable cells were treated with proteasome inhibitor, MG132, there was an even greater reduction in fragment levels. This could be the result of two possibilities; 1) MG132 treatment might inhibit the proteasome which is responsible for PS2 endoproteolysis; or 2) MG 132 inhibits another factor involved in cleaving PS2.

First, to rule out that MG132 inhibits another factor involved in PS cleavage, PS1 and PS2 inducible cells were treated with a series of different known proteasome inhibitors including clasto-lactacystin p-lactone, ALLN, epoxomicin, MG132, and MG262. Subsequent immunoblot analysis of PS proteins with anti-PS antibodies indicated that both PS1 NTF levels and PS2 NTF levels were reduced in cells treated with all of these proteasome inhibitors (FIG. 7A). While each inhibitor had varying effects on the extent of reduction, PS1 NTF levels were reduced by an average of 1.3-fold and PS2 NTF levels were reduced by an average of 1.4-fold. At first glance the fold reduction appears modest; however, considering that degradation of the PS fragments by the proteasome has also been blocked and that fragment levels are tightly regulated, the reduction in PS fragment levels appears significant. Furthermore, it was considered unlikely that all six proteasome inhibitors used here caused nonspecific inhibition of an unknown factor involved in PS endoproteolysis. Instead, it was considered that the most straightforward interpretation of the results is that, in accord with the mode of action the drugs, they all inhibit the proteasome, which is involved in PS endoproteolysis.

To directly address whether the proteasome is responsible for endoproteolysis of PS2, another time course experiment utilizing proteasome inhibitors was devised. MG132 reversibly inhibits the proteasome providing a method by which to first inhibit the proteasome, then remove inhibition and observe whether there is an increase in PS fragment production over time. An increase in PS fragment levels after removal of the proteasome inhibitor would suggest that proteasomes are responsible for endoproteolysis. If by this treatment, no change in PS fragment levels were observed, it would suggest that the proteasome is not involved and instead would imply that PS fragments had already reached saturation. PS2 stable cells were treated with MG132; next, the MG132 was either left to incubate with the cells or was washed off the cells and replaced by fresh media. CHX was added at the time MG132 was removed, to inhibit protein translation, and lysates were collected at two hour intervals for 12 hours. 10% SDS-PAGE and immunoblots using the appropriate antibodies were used to detect PS2 fragment levels. In the cells that were incubated in MG132 for the continuous duration of the experiment, PS2 NTF levels remained relatively constant (FIG. 7B, top panel, left). By contrast, cells in which MG132 was washed out displayed increasing accumulation of PS2 NTF with time (FIG. 7B, top panel, right). As expected, in the same lysates, the cell cycle protein, p27, remained relatively constant when MG132 was present throughout the incubation period whereas its levels decreased over time after MG132 had been washed out, consistent with recovery of proteasome activity. The increase in PS2 NTF levels is most evident when the time points are compared between the cells that were left continuously in MG132 and those in which the drug was removed. For example, at 0 hours, the NTF levels were approximately the same in the MG132 treated samples versus the samples that were treated and then washed free of inhibitor (compare lane 1 to 8). Yet 12 hours later, NTF levels rose 1.8-fold in the cells that had been washed free of MG 132 (compare lane 7 to 14), while the MG132-treated samples remained steady and low (compare lane 1 to 7). A similar trend was observed for the PS2 CTF (data not shown). An anti-actin immunoblot showed that equal amounts of protein lysates were loaded.

Because MG132 has been shown to also inhibit cathepsins and calpains, the experiment was repeated using a more potent and specific proteasome inhibitor, MG262 (FIG. 7B, second panel). Again, similar trends were observed, namely that as the proteasomes recover activity after removal of MG262, there is a notable increase in PS2 fragments. These results suggest that the proteasome might function as the “presenilinase” and at the very least it is somehow involved in the generation of PS2 NTF and CTF.

Discussion

PS endoproteolysis is proposed to be required for the maturation of PS proteins from the unstable Full Length to the stable fragments that are part of the active y-secretase complex [25]. It was found herein that that overexpression of UBQLN, a PS-interactor, decreases PS NTF and CTF levels, likely by blocking access of the presenilinase to the cleavage site, thus preventing endoproteolysis.

Overexpression of UBQLN is associated with greater reduction in PS2 fragments than with PS1 fragments. One explanation for this is that in yeast two-hybrid studies UBQLN1 interacted more strongly with PS2 than PS1 [43]. Moreover, both UBQLN1 and UBQLN2 increase FL PS2 levels more than FL PS1 levels in cotransfection experiments [48]. Therefore, there is a direct correlation between strength of UBQLN interaction and decreased PS fragment production. Nonetheless, both UBQLN1 and UBQLN2 decrease PS1 fragment levels suggesting the two proteins most likely have similar cellular functions, especially in regards to modulation of PS levels.

Since UBQLN has domains that are associated with the UBQLN proteasome pathway it seemed possible that UBQLN decreased fragment levels by perhaps escorting PS fragments to the proteasome for degradation. However, upon proteasomal inhibition, PS fragment levels remained low suggesting that UBQLN did not function in this manner. The more simplistic hypothesis was that UBQLN decreased PS fragments merely by blocking access of the presenilinase. Results of CHX treatment of cells suggested that UBQLN slows the rate of PS2 fragment production. Reduction of UBQLN proteins through siRNA indicated that UBQLN protein knock-down caused the opposite effect of overexpression, an increase in both PS1 and PS2 fragment levels. Finally, while attempting to discern UBQLN's mechanism of action with respect to PS fragment production an interesting observation was made. Namely, when proteasome activity was inhibited an even greater reduction in PS fragment accumulation was observed, which was especially evident without overexpressing UBQLN (data not shown). Conversely, when proteasome inhibition was relieved, PS fragment levels increased. Taken together, the results discussed herein are most consistent with the idea that the proteasome is involved in PS endoproteolysis by either acting directly acts as the presenilinase or it acts indirectly by regulating the presenilinase. The results shown herein indicate that the proteasome is actually involved in the cleavage process, especially because the effects we observed on PS fragment production was observed in six different proteasome inhibitors. In addition, the proteasome has been implicated in endoproteolysis of other proteins [33]. Since UBQLN has domains that are associated with the ubiquitin-proteasome pathway of degradation it is interesting to speculate that UBQLN might act as a tether between PSs and presenilinase activity of the proteasome. Cleavage most likely occurs when PS is embedded in the ER membrane because the large PS loop is oriented towards the cytoplasm where both UBQLN and proteasomes are found. PS cleavage into its fragments occurs early in its maturation process further suggesting that this event occurs when PS is in the ER. Evidence suggests that γ-secretase activity is comprised of PS NTF and CTF fragments rather than the full-length form of the protein. Interestingly, UBQLN modulates both FL PS and fragment levels by increasing FL and decreasing fragment levels. Collectively the data discussed herein suggests that high levels of UBQLN likely reduce y-secretase activity by decreasing formation of PS fragments. This hypothesis is further supported by evidence that overexpression of UBQLN leads to destabilization of both Pen-2 and Nicastrin, two components that are essential for γ-secretase activity.

The ensuing disclosure more specifically relates to suppression of glutamine-induced toxicity in cells, e.g., by methods for inducing increased levels of ubiquilin to reduce aggregation of polyglutamine expansion proteins known to cause cell toxicity and cell death in subjects suffering from neurodegenerative diseases, such as Huntington's and Alzheimer's diseases.

The polynucleotide sequences of the present invention can be obtained using standard techniques known in the art (e.g., molecular cloning, chemical synthesis) and the purity can be determined by polyacrylamide or agarose gel electrophoresis, sequencing analysis, and the like. Polynucleotides also can be isolated using hybridization or computer-based techniques that are well known in the art. Such techniques include, but are not limited to: (1) hybridization of genomic DNA or cDNA libraries with probes to detect homologous nucleotide sequences; (2) antibody screening of polypeptides expressed by DNA sequences (e.g., using an expression library); (3) polymerase chain reaction (PCR) of genomic DNA or cDNA using primers capable of annealing to a nucleic acid sequence of interest; (4) computer searches of sequence databases for related sequences; and (5) differential screening of a subtracted nucleic acid library. Thus, to obtain other receptor encoding polynucleotides, such as those encoding CD4, for example, libraries can be screened for the presence of homologous sequences.

The polynucleotides of the present invention can, if desired: be naked or be in a carrier suitable for passing through a cell membrane (e.g., polynucleotide-liposome complex or a colloidal dispersion system), contained in a vector (e.g., retrovirus vector, adenoviral vectors, and the like), linked to inert beads or other heterologous domains (e.g., antibodies, ligands, biotin, streptavidin, lectins, and the like), or other appropriate compositions disclosed herein or known in the art. Thus, viral and non-viral means of polynucleotide delivery can be achieved and are contemplated.

The polynucleotides of the present invention can also be modified, for example, to be resistant to nucleases to enhance their stability in a pharmaceutical formulation. The described polynucleotides are useful for encoding ubiquilin, especially when such polynucleotides are incorporated into expression systems disclosed herein or known in the art. Accordingly, polynucleotides including an expression vector are also included.

For propagation or expression in cells, polynucleotides described herein can be inserted into a vector. The term “vector” refers to a plasmid, virus, or other vehicle known in the art that can be manipulated by insertion or incorporation of a nucleic acid. Such vectors can be used for genetic manipulation (i.e., “cloning vectors”) or can be used to transcribe or translate the inserted polynucleotide (i.e., “expression vectors”). A vector generally contains at least an origin of replication for propagation in a cell and a promoter. Control elements, including promoters present within an expression vector, are included to facilitate proper transcription and translation (e.g., splicing signal for introns, maintenance of the correct reading frame of the gene to permit in-frame translation of mRNA and stop codons). In vivo or in vitro expression of the polynucleotides described herein can be conferred by a promoter operably linked to the nucleic acid. “Promoter” refers to a minimal nucleic acid sequence sufficient to direct transcription of the nucleic acid to which the promoter is operably linked (see, e.g., Bitter et al., Methods in Enzymology, 153:5 16-544 (1987)). Promoters can constitutively direct transcription, can be tissue-specific, or can render inducible or repressible transcription; such elements are generally located in the 5′ or 3′ regions of the gene so regulated.

The present invention provides compositions comprising a nucleotide sequence encoding for ubiquilin. This composition may be administered with other active agent(s) that have proven to be effective with treatment of HD. The ubiquilin encoding nucleotide sequence can be administered simultaneously with or separately from the other active agent. Doses to be administered are variable according to the treatment period, frequency of administration, the host, and the nature and severity of the disease. The dose can be determined by one skilled in the art without an undue amount of experimentation.

The composition of the invention is administered in substantially non-toxic dosage concentrations sufficient to ensure the expression of a sufficient amount of ubiquilin to provide the desired reduction of cell death and/or toxicity, or reduced aggregation of polyglutamine expanding Htt proteins. The actual dosage administered will be determined by physical and physiological factors such as age, body weight, severity of condition, and/or clinical history of the patient.

The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC ₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The compositions of the present invention may comprise both the above-discussed components together with one or more acceptable carriers thereof and optionally other therapeutic agents. Each carrier must be “pharmaceutically acceptable” in the sense of being compatible with the other ingredients of the formulation and not injurious to the subject.

The present invention provides expression vectors comprising polynucleotide that encode ubiquilin peptides. The expression vectors of the invention may comprise polynucleotides operatively linked to an enhancer-promoter, that being either a prokaryotic or eukaryotic promoter depending on host cells..

As used herein, the phrase “enhancer-promote” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

An enhancer-promoter used in a vector construct of the present invention can be any enhancer-promoter that drives expression in a cell to be transfected. By employing an enhancer-promoter with well-known properties, the level and pattern of gene product expression can be optimized. A coding sequence of an expression vector is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (RNA). Transcription-terminating regions are well known in the art. A preferred transcription-terminating region is derived from a bovine growth hormone gene.

Preferably, expression vectors of the present invention comprise polynucleotides that encode polypeptides comprising the amino acid residue sequence of ubiquilin. Further, an expression vector can include longer nucleotide sequences that can code larger polypeptides or peptides which nevertheless include the basic coding region. In any event, it should be appreciated that due to codon redundancy as well as biological functional equivalence, this aspect of the invention is not limited to the particular DNA molecules corresponding to the polypeptide sequences noted above.

Exemplary vectors include the mammalian expression vectors of the pCMV family including pCMV6b and pCMV6c (Chiron Corp., Emeryville Calif.) and pRc/CMV (Invitrogen, San Diego, Calif.). In certain cases, and specifically in the case of these individual mammalian expression vectors, the resulting constructs can require co-transfection with a vector containing a selectable marker such as pSV2neo. Via co-transfection into a dihydrofolate reductase-deficient Chinese hamster ovary cell line, such as DG44, clones expressing a ubiquilin peptide by virtue of DNA incorporated into such expression vectors can be detected.

A DNA molecule of the present invention can be incorporated into a vector using a number of techniques which are well known in the art. For instance, the vector pUC18 has been demonstrated to be of particular value. Likewise, the related vectors M13mp18 and M13mp19 can be used in certain embodiments of the invention, in particular, in performing dideoxy sequencing.

An expression vector of the present invention is useful both as a means for preparing quantities of ubiquilin encoding DNA itself, and as a means for preparing the encoded peptides. It is contemplated that where ubiquilin peptides of the invention are made by recombinant means, one can employ either prokaryotic or eukaryotic expression vectors as shuttle systems. Such a system is described herein which allows the use of bacterial host cells as well as eukaryotic host cells.

Where expression of recombinant polypeptide of the present invention is desired and a eukaryotic host is contemplated, it is most desirable to employ a vector, such as a plasmid, that incorporates a eukaryotic origin of replication. Additionally, for the purposes of expression in eukaryotic systems, one desires to position the ubiquilin peptide encoding sequence adjacent to and under the control of an effective eukaryotic promoter such as promoters used in combination with Chinese hamster ovary cells. To bring a coding sequence under control of a promoter, whether it is eukaryotic or prokaryotic, what is generally needed is to position the 5′ end of the translation initiation side of the proper translational reading frame of the polypeptide between about 1 and about 50 nucleotides 3′ of or downstream with respect to the promoter chosen. Furthermore, where eukaryotic expression is anticipated, one would typically desire to incorporate into the transcriptional unit which includes the ubiquilin peptide, an appropriate polyadenylation site.

The pRc/CMV vector (available from Invitrogen) is an exemplary vector for expressing ubiquilin peptide in mammalian cells, particularly COS and CHO cells. A polypeptide of the present invention under the control of a CMV promoter can be efficiently expressed in mammalian cells. The pCMV plasmids are a series of mammalian expression vectors of particular utility in the present invention. The vectors are designed for use in essentially all cultured cells and work extremely well in SV40-transformed simian COS cell lines. The pCMV1, 2, 3, and 5 vectors differ from each other in certain unique restriction sites in the polylinker region of each plasmid. The pCMV4 vector differs from these 4 plasmids in containing a translation enhancer in the sequence prior to the polylinker. While they are not directly derived from the pCMV1-5 series of vectors, the functionally similar pCMV6 b and c vectors are available from the Chiron Corp. of Emeryville, Calif. and are identical except for the orientation of the polylinker region which is reversed in one relative to the other.

In yet another embodiment, the present invention provides recombinant host cells transformed or transfected with a polynucleotide that encodes a ubiquilin peptide, as well as transgenic cells derived from those transformed or transfected cells. Means of transforming or transfecting cells with exogenous polynucleotide such as DNA molecules are well known in the art and include techniques such as calcium-phosphate- or DEAE-dextran-mediated transfection, protoplast fusion, electroporation, liposome mediated transfection, direct microinjection and adenovirus infection.

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

In the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

A transfected cell can be prokaryotic or eukaryotic. Preferably, the host cells of the invention are eukaryotic host cells.

In addition to prokaryotes, eukaryotic microbes, such as yeast can also be used to produce increased levels of the ubiquilin peptide. Saccharomyces cerevisiae or common baker's yeast is the most commonly used among eukaryotic microorganisms, although a number of other strains are commonly available.

In addition to microorganisms, cultures of cells derived from multicellular organisms can also be used as hosts. In principle, any such cell culture is workable, whether from vertebrate or invertebrate culture. However, interest has been greatest in vertebrate cells, and propagation of vertebrate cells in culture (tissue culture) has become a routine procedure in recent years. Examples of such useful host cell lines are AtT-20, VERO and HeLa cells, Chinese hamster ovary (CHO) cell lines, and W138, BHK, COSM6, COS-1, COS-7, 293 and MDCK cell lines. Expression vectors for such cells ordinarily include (if necessary) an origin of replication, a promoter located upstream of the gene to be expressed, along with any necessary ribosome binding sites, RNA splice sites, polyadenylation site, and transcriptional terminator sequences.

For use in mammalian cells, the control functions on the expression vectors are often derived from viral material. For example, commonly used promoters are derived from polyoma, Adenovirus 2, Cytomegalovirus and most frequently Simian Virus 40 (SV40). The early and late promoters of SV40 virus are particularly useful because both are obtained easily from the virus as a fragment which also contains the SV40 viral origin of replication. Smaller or larger SV40 fragments can also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

A transfected cell can also serve as a carrier. By way of example, a liver cell can be removed from an organism, transfected with a polynucleotide of the present invention using methods set forth above and then the transfected cell returned to the organism (e.g. injected intravascully).

The features and advantages of the invention are more fully apparent from the following illustrative examples, which are not intended in any way to be limitingly construed, as regards the invention hereinafter claimed.

### EXAMPLES

Methods and Materials

Cell Culture, DNA Transfection, Immunofluorescence and Electron Microscopy

HeLa cells were grown in DME supplemented with 10% FBS. Cells were transiently transfected with plasmid DNA using by calcium phosphate coprecipitation. Stable HeLa cell lines were isolated by cotransfection of EGFP expression constructs with a pNeo plasmid and selection with G418. Stable cell lines were identified by GFP-fluorescence.

Immunofluorescence staining and fluorescence images of fixed and live cells and animals were captured on a Zeiss Axiovert 100 microscope using a Hammatsu camera using C-25 Imaging software.

Protein preparation, SDS-PAGE, and immunoblotting

Cell and tissue protein lysates were prepared as described previously [41a]. Standard protocol for protein separation and immunoblotting was followed [41a]. Antibodies against GFP and C. elegans ubiquilin were prepared by injecting rabbits with purified GST-GFP fusion protein, or with a KHL-conjugated peptide corresponding to inferred residues 7-30 of the ubiquilin ORF, respectively. The anti-ubiquilin monoclonal antibody used was described previously [43a].

Quantification of Cell Death

Cell death was monitored either through examination of the nuclear morphology observed after Hoechst 33342 (1 μg/ml) staining, or through detecting the membrane selective permeability following 3 μM of propidium iodide (PI) staining. Terminal deoxynucleotidyl Transferase Biotin-dUTP Nick End Labeling (TUNEL) staining was performed with DeadEnd Colorimetric TUNEL staining kits purchased from Promega. For detecting caspase-3 activation, two methods were used. Cell lysates were separated by SDS-PAGE and probed with an antibody against the cleaved substrate for caspase-3. Alternatively, caspase activity was also detected by flurogenic techniques by incubating the cell lysates with a flurogenic caspase-3 substrate, AC-DEVD-AMC in a 96-well plate. After incubation, the cleaved free AMC was scanned by a fluorescence multi-well plate reader (SOSTmax, Sunnyvale, CA) with an excitation at 380 nm (excitation) and emission at 460 nm (emission).

RNAi Studies

Stable expression EGFP-Q74 cell line was plated in 24 well-plate (Costar) in a low cell density. After 24 hours following the plating, cells were transfected with SMARTpools of ubiquilin-1 and -2 siRNAs [43a], RISC-free SMARTpool control siRNAs [43a], or mock transfection by just adding the transfection reagent according to the suggested protocol by the company. For comparing cell growth, phase contrast microscopy was performed just before siRNA transfection and 4 days after the transfection. For detecting cell death, cells were collected for detecting the activity of caspase-3 on 4 days after the transfection, or stained with PI, Hoechst 33342, or TUNEL method on 5 days following the transfection.

Molecular Cloning and Expression

Expression of human ubiquilin-1 cDNA was described previously [43a]. The cDNA encompassing the entire open reading frame (ORF) of C. elegans ubiquilin fusing it downstream of monomeric RFP (mRFP) was cloned, under the control of a CMV promoter. The EGFP-Htt-Exon polyglutamine fusion proteins were kindly provided by Dr. David C. Rubinsztein and were cloned in the pEGFP vector [45a]. The various GFP constructs were subcloned into a plasmid containing the unc54 promoter for expression in C. elegans [27a]. For the RNAi experiments in C. elegans, the complete ubiquilin ORF of C. elegans was cloned into the RNA interference (RNAi) vector described previously [28a]. The RNAi bacterial feeding protocol that was followed was also essentially as described by those authors [28a, 29a].

C. elegans Experiments

The standard microinjection procedure to derive stable C. elegans lines was used [46]. The standard procedure for growth and maintenance of all C. elegans lines was also utilized.

Expression of Htt Exon with Polyglutamine Repeats

Previous studies had indicated that the NH₂-terminal portion of Htt protein, containing the expanded polyglutamine tract in HD, is likely to be involved in HD pathogenesis. Moreover, expression of the NH₂-terminal Htt fragment containing the pathogenic range of polyglutainine repeats in cells is associated with several HD-associated manifestations, including the formation of intracellular aggregates and cell death [15a, 16a]. Accordingly, a useful method to study the aggregation and toxicity of polyglutamine proteins is to express Htt exon-1 fragments with different numbers of polyglutamine repeats as GFP-fusion proteins, and examine phenotypes of the cells and animals that express the proteins using GFP as a reporter. This approach was utilized to study the effects of overexpression as well as loss of ubiquilin expression on Htt-exon-1 polyglutamine-induced toxicity in cell and animal models of HD.

Consistent with previous findings of the present inventors it was found that transient transfection of HeLa cells with GFP-fusion proteins of Htt-exon-1 containing polyglutamine repeats in the pathological range of 55 and 72 induced a length-dependent increase in cell death compared to cells expression GFP-Htt-exon-1Q28, a non-pathogenic range of polyglutamine repeats, or GFP alone (FIG. 8B). Immunoblot analysis using an anti-GFP antibody confirmed that proteins of the expected size were expressed in every case (FIG. 8A). Furthermore, examination of transiently transfected HeLa cells by immunofluorescence microscopy revealed a strong positive correlation in the number of inclusions (both nuclear and cytoplasmic) that formed in GFP-fluorescent cells and constructs expressing longer lengths of polyglutamine tracts (data not shown). Biochemical fractionation of transfected HeLa cells into Triton-X100 soluble and insoluble material revealed a similar trend, showing that as the length of polyglutamine repeats increased the percentage of the full-length GFP-expressed polypeptides that became insoluble progressively increased (FIG. 8C and D).

Having established that expression of GFP-tagged Htt-Exon1-polyglutamine fusion proteins in HeLa cells caused a polyglutamine-number dependent increase in intracellular inclusion formation, Triton-X100 insolubility, and cell death, it was necessary to examined what effect overexpression of ubiquilin-1 has on these properties. For these studies, only the GFP-Htt Exon 1 polyQ74 construct was concentrated on because it induced the most robust effects on all the three properties described. Interestingly, cotransfection of increasing amounts of full-length untagged ubiquilin-1 CDNA expression construct with a constant amount of the GFP-Htt Exon 1 Q74 construct resulted in dose-dependent increase in accumulation of GFP-HttPolyQ74 fusion protein, according to anti-GFP immunoblot analysis of equal amounts of protein lysate prepared from the transfected cells (FIG. 9A). The increase in accumulation of GFP-HttPolyQ74-fusion protein, propagated by cotransfection of ubiquilin-1 expression construct, provided a platform to address an important issue regarding the toxicity of polyglutamine proteins. Namely, does higher levels of GFP-HttPolyQ74-fusion protein accumulation increase cell death, or increase the propensity of the protein to become more insoluble, result in formation of more numerous, or larger, intracellular inclusions? As discussed hereinabove, there is currently considerable debate regarding the toxicity of polyglutamine proteins regarding these issues. In particular, some studies suggest that toxicity of expanded polyglutamine proteins is governed by the amount of polyglutamine protein that is soluble and not by the portion that is aggregated, whereas other have reached the opposite conclusion. Moreover, these and other studies suggest that elevated levels of polyglutamine proteins should increase cell death. Surprisingly, it was found herein that the cells transfected with increasing amounts of ubiquilin-1 cDNA expression construct displayed a dose-dependent decrease in cell death compared to cells transfected with GFP-Htt Exon-1PolyQ74 alone (FIG. 9B). In addition, biochemical fractionation of a similar set of transfected cells revealed that although ubiquilin propagated a dose-dependent increase in GFP-HttPolyQ74 protein accumulation, a greater proportion of the GFP-HttPolyQ74-fusion protein remained soluble, suggesting that ubiquilin overexpression reduces intracellular aggregate formation (FIG. 9C and D). Taken together these studies indicate that overexpression of ubiquilin-1 increases GFP-HttPolyQ74 protein levels and reduces polyglutamine-induced cell death and protein-aggregation at least when assayed using HeLa cells.

Overexpression of Ubiquilin in Neuronal Cells

Next it was investigated whether the modulation of polyglutamine aggregation and toxicity by ubiquilin-1 overexpression is manifested in neuronal cells, which might be more relevant in terms of controlling neurodegeneration in HD and related disorders. As such, the above experiments were repeated using primary mouse primary neuronal cultures. As shown in FIG. 10A-C the results obtained were similar, namely ubiquilin-1 overexpression decreased GFP-HttPolyQ74-induced neuronal cell death, which was associated with decreased inclusion formation. The insolubility of the Htt protein was not measured in these cells due difficulties in preparing reliable soluble and insoluble material from neurons and because of a limitation in the number for cells required for the assay.

Establishment of HeLa Cell Lines

To obtain further evidence that ubiquilin protects cells against polyglutamine-induced protein aggregation and cytotoxicity, HeLa cell lines were established that stably expressed either GFP alone, GFP-HttPolyQ28, GFP-HttPolyQ55, or GFP-HttPolyQ74 fusion proteins. FIG. 11A shows an immunoblot of the relative expression levels of the different GFP-containing proteins in protein lysates from representative examples of the cell lines. Two of these cell lines were used, GFP-HttPolyQ28-2 and GFP-HttPolyQ74-3, to determine if the length of polyglutamine expression affects the vulnerability (sensitivity) of cells to different stress-inducing agents, and whether ubiquilin can protect against the cytotoxic effects of these agents. As shown in FIG. 11B, although the stable GFP-HttQ28-1 and GFP-HttQ74-3 cell lines exhibited strong GFP fluorescence in both the cytoplasm and nucleus, foci of fluorescence were only found in the latter line. This is in accord with results obtained by transient transfection of the constructs, showing that GFP-fusion proteins containing polyglutamine repeats of 40 or more are more likely to aggregate than proteins with shorter repeats (data not shown). However, the presence of the GFP-containing aggregates in the GFP-HttQ74-3 cell line (as well as other GFP-HttQ74 and GFP-HttQ55 cell lines) clearly demonstrates that aggregates, by themselves, are not sufficient to induce cytotoxicity, otherwise the cell lines would not be viable. It is possible that the cell lines containing polyglutamine aggregates might have induced compensatory mechanisms to overcome the toxicity induced by the expression of the polyglutamine proteins, and/or that the selection pressure used to isolate the cell lines establishes a threshold of polyglutamine expression that is just tolerated by the cells.

Sensitivity of Cell Lines

The sensitivity of the cell lines was tested relative to low levels of H₂O₂, which is known to induce oxidative stress. Additionally, the cells were starved for serum for 5 hours as an additional method to assess the sensitivity of the cells to oxidative stress [17a -22a]. Wild type HeLa cells are normally insensitive to a 5 hours exposure of H₂O₂ concentrations of 200 uM, or lower (data not shown). The GFP-HttQ28-2 cell line exhibited similar insensitivity to H₂O₂ concentrations within this same range. Remarkably, however, the GFP-HttQ74-3 cell line was acutely sensitive to H₂O₂ concentrations of as little as 100 uM, inducing approximately 30% of cell death after this treatment (FIG. 11D). Treatment of the GFP-HttQ74-3 with 150 and 200 uM lead to a further dose-dependent increase in cell death (FIG. 11D), suggesting that expression of the GFP-Htt protein containing 74 polyglutainine repeats sensitizes cells to oxidative stress. To examine if ubiquilin exerts any protective effect on polyglutamine-induced cell death, the GFP-HttQ28-2 and GFP-HttQ74-3 stable cell lines were transiently transfected with a ubiquilin-1 expression plasmid by the calcium phosphate coprecipitation procedure. For these experiments, the cells were starved for serum as an independent method to assess the sensitivity of the cells to oxidative stress [22a]. As shown in FIG. 11E-G, there was no significant difference in cell death between the mock-transfected and ubiquilin-1-transfected GFP-HttQ28-2 cell line. By contrast, mock transfection of the GFP-HttQ74-3 cell line induced significant cell death (FIG. 11E and G), which is consistent with increased sensitivity of the cells to oxidative stress caused by the serum withdrawal. More significantly, overexpression of ubiquilin-1 reduced less cell death under similar circumstances (FIG. 11E-G). Thus 24 hours after transfection and serum deprivation, approximately 50% reduction in cell death was observed in the GFP-HttQ74-3 cell line transfected with ubiquilin-1 compared to untransfected cells (FIG. 11G). Moreover, in further experiments it was found that transfection of increasing amounts of ubiquilin-1 expression plasmid resulted in a dose-dependent protection against cell death in the GFP-HttQ74-3 cell line (FIG. 11H and I). These results, taken together, suggest that overexpression of ubiquilin-1 reduces sensitivity of cell to stress induced by expanded polyglutamine proteins.

Effects of Reduction of Endogeneous Ubiquilin

To confirm the protective role of ubiquilin in expanded polyglutamine-induced cell death, the consequence of reducing endogenous ubiquilin protein levels in the GFP-HttQ74-3 cell line was studied. Because human ubiquilin-1 and ubiquilin-2 share 80 % sequence identity [31 a], it was decided to knockdown expression of the both proteins using siRNA SMARTpools directed against both genes. Two days after transfection of the GFP-HttQ74-3 cell line with the pooled siRNAs, both ubiquilin-1 and -2 protein levels were reduced by >90%, and this low level of ubiquilin was maintained for at least 4 days after transfection (FIG. 12A). During the first 2 days after transfection of the ubiquilin siRNA no obvious morphological change was noticed in the GFP-HttQ74-3 expressing cells. However, 3 days after transfection, it became visually obvious that the cells transfected with ubiquilin siRNA had not proliferated compared to cells plated from the same cell line that were either mock transfected or transfected with a control siRNA pool specifically designed not to target any known gene (FIG. 12B). Moreover, 4 days after ubiquilin siRNA transfection, a significant increase in cell death was observed in the GFP-HttQ74-3 expressing cells, which was detected by nuclear fragmentation/condensation (FIG. 12C and 5D), TUNEL staining (FIG. 12E and 5F), propidium iodide (PI) staining (FIG. 12G) and activation of caspase-3 (FIG. 12H and 5I). In addition, a filter retardation assay demonstrated that siRNA knockdown of ubiquilin levels leads to accumulation of expanded GFP-immunoreactive aggregates (FIG. 12J and 12K). None, of these phenomena were observed after mock transfection or control siRNA transfection of GFP-HttQ74-3 cells or after siRNA knockdown of ubiquilin in normal HeLa cells (data not shown). These results demonstrate that down-regulation of ubiquilin levels halts cell proliferation, triggers cell death and the accumulation of polyglutamine aggregates in the GFP-HttQ74-3 expressing cell line.

Suppression of Polyglutamine-Induced Protein Toxicity in Animals

To investigate this polyglutamine-induced protein toxicity, the nematode, Caenorhabditis elegans (C. elegans), was used to model polyglutamine-protein aggregation and toxicity. Previous studies have shown that muscle-specific expression of GFP-polyglutamine fusion proteins in C. elegans induces polyglutamine length-dependent protein aggregation and a decrease in animal motility [23a-26a]. The same muscle specific unc-54-promoter [27a] was used to drive expression of the different GFP-fusion constructs (described above) in C. elegans and established several stable lines that expressed each of the different proteins. An immunoblot of protein extracts prepared from the animals confirmed that GFP-fusion proteins of the appropriate size were expressed in each case (FIG. 13A). In accord with previous findings, it was found that expression of the different GFP-fusion proteins resulted in polyglutamine length-dependent change in GFP fluorescence in the muscle cells of the animals changing, from diffuse fluorescence to more compact foci as the length of polyglutamine repeats increased. Examples of low and high magnifications images taken of animals expressing the different GFP-fusion proteins are shown in FIG. 13B and C, respectively. It was examined whether muscle function was altered in these animals by counting the number of body bends flexed by the worms over a one-minute interval of continuous movement, which has been shown to correlate well with C. elegans motility. Consistent with a previous report [24a] it was found that 1 day-old adult nematodes that expressed GFP-Htt-polyQ fusion proteins displayed a polyglutamine length-dependent decrease in body movement compared to wild type animals (FIG. 13D). Having established suitable GFP-expressing C. elegans stable lines, next the effects of reducing ubiquilin levels in the animals was determined. To knockdown ubiquilin protein in the nematodes the complete C. elegans ubiquilin cDNA (C. elegans has only one ubiquilin gene, F15C11.2a) was cloned into the bacterial RNA interference (RNAi) expression vector L4440 [28a]. The L4440 vector is widely used to genetically disrupt expression of a particular C. elegans gene by letting nematodes feed on bacteria that are transformed with the plasmid containing the cloned cDNA of a gene being targeted for RNAi. When induced with IPTG, the bacteria synthesize a dsRNA copy of the cDNA, which when ingested by the nematodes, frequently induces genetic interference throughout the body of the worm. Accordingly, bacteria that were transformed was fed with either the L4440 vector alone, or L4440 vector containing the cloned C. elegans ubiquilin cDNA (L4440:ubiquilin), or L4417, a related vector containing a cloned GFP cDNA (L4417:GFP) [29], that were either exposed to IPTG or not, to GFP-, GFP-HttPolyQ28-, and GFP-HttPolyQ74-expressing nematodes. After being fed for two days, the number of body bends flexed by the worms was counted during periods of continuous movement on agar plates over a one-minute period. GFP-HttPolyQ28- and GFP-HttPolyQ74-expressing adult nematodes that had fed on Isopropyl-13-D-thiogalactopyranoside (IPTG) exposed bacteria containing L4417:GFP plasmid had increased number of body bends compared to similar stage animals that fed on IPTG-exposed bacteria containing the L4440 vector alone (FIG. 14A). In fact, the number of body bends in GFP-HttPolyQ28- and GFP-HttPolyQ74-expressing animals was restored to within 16% and 25%, respectively, of the level found in normal wild type animals, suggesting that genetic interference of GFP in adult C. elegans can restore some, but not all of the crippling effects produced by expression of the GFP-HttPolyQ fusion proteins in muscle cells. An examination of the rescued animals by fluorescence microscopy revealed almost complete loss of GFP fluorescence (data not shown), which is consistent with successful dsRNA interference of GFP. The loss of GFP fluorescence after RNAi suggests that GFP-polyQ-fusion proteins in GFP-HttPolyQ28- and GFP-HttPolyQ74-expressing animals are dynamic and undergoing constant turnover.

Having established that the bacterial feeding protocol was suitable for silencing GFP in the polyglutamine expressing C. elegans lines, next the procedure to genetically interfere with expression of endogenous ubiquilin in the different lines was used. Interestingly, wild type nematodes that were fed IPTG exposed bacteria containing L4440:ubiquilin plasmid had approximately 5% fewer body bends compared to similar stage worms that were fed IPTG exposed bacteria containing the L4440 vector alone, suggesting that RNA interference of ubiquilin in the absence of GFP-Htt-polyglutamine expression might compromise worm movement to some degree (FIG. 14B). The reduction in body bends was specific to worms that had fed on bacteria containing L4440:ubiquilin plasmid exposed to IPTG, the dsRNA interference inducer, but not in its absence, as expected. More interestingly, GFP-HttPolyQ28-and GFP-HttPolyQ74-expressing nematodes that fed on IPTG exposed bacteria containing L4440-ubiquilin cDNA propagated 55% and 75% fewer body bends, respectively, compared to siblings that had fed on IPTG exposed bacteria containing the L4440 vector alone. The reduction in the number of body bends in the worms appeared to be a consequence of dsRNA interference of ubiquilin, because it was only observed in nematodes that fed on IPTG exposed bacteria containing L4440 plasmid containing cloned ubiquilin cDNA, and was neither seen if the cDNA was absent, nor if the gratuitous inducer was omitted. Together these results suggest that genetic interference of C. elegans ubiquilin reduces worm motility and that movement is even more compromised in animals expressing longer lengths of GFP-Htt-polyglutamine fusion proteins. Finally, protein extracts from worms recovered from the different treatments was immunoblotted and it was found that the levels of ubiquilin protein in the worms was indeed reduced in only the worms that were fed bacteria containing L4440:ubiquilin plasmid exposed to IPTG (data not shown).

Rescue by Overexpression of Ubiquilin

Because dsRNA interference of ubiquilin worsened body bend movement in polyglutamine expressing nematodes it was questioned if overexpression of ubiquilin would prevent (rescue) the GFP-Htt-polyglutamine-dependent loss of movement. To investigate this possibility, the C. elegans ubiquilin was tagged with the monomeric red fluorescent protein (mRFP), using the latter as a reporter of transgenic ubiquilin expression. Immunofluorescence microscopy of HeLa cells transfected with the mRFP-ubiquilin fusion construct revealed a pattern of red fluorescent that was similar to antibody staining of endogenous and transfected human ubiquilin proteins in the cells, suggesting that the mRFP-tag does not interfere with localization of the C. elegans ubiquilin protein (FIG. 14C). Furthermore, immunoblot analysis of protein lysates prepared from the transfected cells confirmed expression of a fusion polypeptide composed of RFP and C. elegans ubiquilin, and it was of the expected size (FIG. 14D).

Next it was determined if forced expression of the ubiquilin-RFP fusion protein in muscle cells of C. elegans affected body bend movement in GFP-HttPolyQ55-expressing nematode lines. GFP-HttPolyQ 55 was chosen because it was intermediate in both number and toxicity of the three different lengths of polyglutamine proteins that had been examined. Accordingly, six new stable GFP-HttPolyQ55 expressing nematode lines were established, three of which were derived by injecting plasmid DNA containing GFP-Htt-Exon1-PolyQ55 placed under the control of the unc-54 promoter, and the other three by injecting a 2:1 DNA ratio of a mixture of unc-54-driven ubiquilin:RFP and unc-54-driven GFP-Htt-Exon1-PolyQ55 plasmids, respectively. All six lines expressed GFP fluorescence (FIG. 15 A-C), whereas RFP fluorescence was only detected in the lines in which RFP-ubiquilin plasmid was coinjected, as expected.

Similar to the phenotype observed previously, muscle-specific expression of GFP-HttPolyQ55 by itself led to -50-60% reduction in body bend movement in the three new nematode lines, compared to similar aged wild type animals. By contrast, all three nematode lines that coexpressed GFP-HttPolyQ55 and mRFP-ubiquilin were less severely affected, with line 1 displaying 28 body bends per minute, line 2 displaying 17 body bends, and line 3 displaying 23 body bends, which represent almost ˜120% to 55% greater movement compared to lines that expressed GFP-HttPolyQ55 alone (FIG. 15D). Closer examination of GFP fluorescence in the coexpressing animals revealed a noticeable difference in the pattern of fluorescence in the three lines: line 1 worms displayed diffuse fluorescence that was similar in many respects to that seen in animals expressing GFP alone (compare FIG. 15Aa and 15Ca with FIG. 13Ba and 13Ca, of low and high magnifications images, respectively), whereas line 2 contained more GFP fluorescent foci, but these were less in number than any of the lines in which GFP-HttPolyQ55 was expressed alone, whereas line 3 had slighter fewer foci than line 2, but more than line 1 (compare GFP foci in FIG. 15A a, d and g). It is speculated that number of GFP foci that formed in the animals is governed by the relative ratio of expression of the GFP- and RFP-fusion proteins. It is important to note that body bend movement in the coexpressing animals showed a negative correlation with the number of GFP foci, suggesting that the foci are detrimental to movement. Interestingly, it was noticed that the RFP fluorescence colocalized with many, if not most, of the GFP foci, particularly compact foci, in the coexpressing animals (FIG. 15C c-e), suggesting that RFP-ubiquilin fusion binds to the polyglutamine GFP-containing aggregates in live animals. Taken together these results indicate that loss of C. elegans ubiquilin expression exacerbates polyglutamine-induced toxicity in muscle cells of the worms and that overexpression of ubiquilin protein can protect the worms against this polyglutamine-induced loss of movement.

The above results clearly demonstrate that overexpression of ubiquilin in cells decreases both aggregation and toxicity of expanded Htt-polyglutamine proteins, whereas a reduction in ubiquilin levels by RNA interference produces the opposite effect. Several of the results support the fact that ubiquilin is regulating aggregation and toxicity of polyglutamine proteins. First, ubiquilin proteins have been shown to bind and colocalize with polyglutamine and the red fluorescence emitted by expression of mRFP-tagged C. elegans ubiquilin-fusion protein colocalized with the green fluorescence of GFP-HttPolyQ55 fusi\' protein in compact foci in transgenic nematode lines. This colocalization, which was detected in living animals, and rules out the possibility that the previous colocalization of ubiquilin and polyglutamine proteins in mouse brain [14a] was an artifact produced by fixation and/or staining. Second, overexpression of ubiquilin-1 reduced GFP-polyglutamine inclusions and protein-aggregation and also suppressed polyglutamine-induced cell death in both HeLa cells and primary cortical neurons. Third, overexpression of ubiquilin-1 suppressed oxidative stress-induced cell death in a stable HeLa cell line expressing GFP-HttPolyQ74 fusion protein. By contrast knockdown of ubiquilin in the cell line was associated with increased DNA fragmentation, caspase activation, GFP-aggregate formation, and cell death. Fourth, RNA interference of the C. elegans ubiquilin gene led to a reduction in body bend movement in nematode lines stably expressing GFP-Htt-fusion proteins. By contrast, coexpression of mRFP-tagged ubiquilin with GFP-HttPolyQ55 fusion protein in the muscle prevented the motility defect seen in nematode lines that expressed the polyglutamine fusion protein alone.

Importantly, that overexpression of ubiquilin suppresses cell death induced by agents that induce oxidative stress. The HeLa cell line that stably expressed the GFP-Htt fusion protein containing 74 polyglutamine residues was highly sensitized to H₂O₂ and serum withdrawal compared to the line expressing only 28 polyglutamine repeats, and overexpression of ubiquilin suppressed the vulnerability of the cells to these agents.

There is evidence that ubiquilin serves to rid cells of misfolded, aggregated and ubiquitinated forms of proteins such as those that form by expanded polyglutamine proteins. Firstly, it was found that overexpression of ubiquilin reduced the amount of GFP-tagged polyglutamine proteins that aggregated in cells, in a dose-dependent manner. The reduction was evident both by fewer number of GFP-HttPolyQ-fusion protein inclusions that formed in neuronal cells and C. elegans lines (data not shown) when higher doses of ubiquilin cDNA was coexpressed in cells and animals compared to when it was not coexpressed, and by a reduction in the amount of GFP-fusion protein that appeared to be aggregated, as revealed by the amount of protein that was trapped in the stacking gel after SDS-PAGE, and following filtration of cell lysates through filters. Conversely, reduction of ubiquilin by RNAi led to an increase in the amount of aggregated GFP-Htt fusion protein in cell lysates. Secondly, it was found that the red fluorescence emitted by the expression of mRFP-tagged ubiquilin protein colocalized with more compact foci of green fluorescence of GFP-HttPolyQ55 fusion protein and less so with the more filamentous, and presumably less aggregated forms of the GFP-HttPolyQ55 fusion protein, indicating that ubiquilin binds and targets more highly misfolded polyglutamine proteins for degradation. Thirdly, in previous studies it was found that ubiquilin colocalized with ubiquitin positive structures in cells particularly in aggresomes, which are structures containing misfolded proteins in cells, and that overexpression of ubiquilin reduced the amount of ubiquitinated PS2 proteins that accumulated in cells [41a].

Interestingly, Doi et al. demonstrated that ubiquilin colocalized with Htt aggregates that were ubiquitin positive in cells and mouse brain [14a]. These observations indicate that ubiquilin binds and targets preferably only misfolded and ubiquitinated Htt proteins for degradation.

A particularly noteworthy property is that ubiquilin overexpression increased the accumulation of total GFP-Htt fusion protein, while decreasing the portion of the protein that formed aggregates in cells. The increase in polyglutamine proteins propagated by overexpression of ubiquilin was associated with decreased cell death. These results suggest that ubiquilin is not only capable of decreasing the number of polyglutamine-protein aggregates in cells but is also capable of increasing the amount of soluble polyglutamine-protein that can accumulate in cells. This result is more compatible with the idea that aggregates and not the soluble forms of expanded polyglutamine proteins are more cytotoxic to cells.

### REFERENCES

The contents of all references cited herein are hereby incorporated by reference herein for all purposes.

References Cited in Disclosure Relating to Ubiquilin Regulation of Presenilin Endoproteolysis

1. Edbauer, D., Winkler, E., Regula, J. T., Pesold, B., Steiner, H. and Hass, C. (2003) Nat. Cell. Biol., 5, 486-8.

2. Kimberly, W. T., LaVoie, M. J., Ostaszewski, B. L., Ye, W., Wolfe, M. S. and Selkoe, D. J. (2003) Proc. Natl. Acad. Sci U. S. A., 100, 6382-7.

3. De Strooper, B. (2003) Neuron 38, 9-12.

4. Haass, C. and Steiner, H. (2002) Trends Cell Biol., 12, 556-62.

5. Takasugi, N., Tomita, T., Hayashi, I., Tsuruoka, M., Niimura, M., Takahashi, Y Thinakaran, G. and Iwatsubo, T. (2003) Nature 422, 438-41.

6. Hu, Y. and Fortini, M. E. (2003) J. Cell Biol., 161, 685-90.

7. Steiner, H., Winkler, E., Edbauer, D., Prokop, S., Basset, G., Yamasaki, A., Kostka, M. and Haass, C. (2002) J. Biol. Chem., 277, 39062-5.

8. Francis, R., McGrath, G., Zhang, J., Ruddy, D. A., Sym, M., Apfeld, J., Nicoll, M., Maxwell, M., Hai, B ., Ellis, M. C., Parks, A. L., Xu, W., Li, J., Gurney, M., Myers, R. L., Himes, C. S., Hiebsch, R., Ruble, C., Nye, J. S. and Curtis, D. (2002) Dev. Cell 3, 85-97.

9. Edbauer, D., Winkler, E., Haass, C. and Steiner, H. (2002) Proc. Natl. Acad. Sci U. S.A., 99, 8666-71.

10. Sastre, M., Steiner, H., Fuchs, K., Capell, A., Multhaup, G., Condron, M. M., Teplow, D. B. and Haass, C. (2001) EMBO Rep., 2, 835-41.

11. LaVoie, M. J., Fraering, P. C., Ostaszewski, B. L., Ye, W., Kimberly, W. T., Wolfe, M. S. and Selkoe, D. J. (2003) J. Biol. Chem., 278, 37213-22.

12. Fraering, P. C., LaVoie, M. J., Ye, W., Ostaszewski, B. L., Kimberly, W. T., Selkoe, D. J. and Wolfe, M. S. (2004) Biochemistry 43, 323-33.

13. Luo, W. J., Wang, H., Li, H., Kim, B. S., Shah, S., Lee, H. J., Thinakaran, G., Kim, T. W., Yu, G. and Xu, H. (2003) J. Biol. Chem. 278, 7850-4.

14. Yang, D. S ., Tandon, A., Chen, F., Yu, G., Y u, H., Arawaka, S., Hasegawa, H., Duthie, M., Schmidt, S. D., Ramabhadran, T. V., Nixon, R. A., Mathews, P. M., Gandy, S. E., Mount, H. T., St George-Hyslop, P. and Fraser, P. E. (2002) J. Biol. Chem.277, 28135-42.

15. Kaether, C., Capell, A., Edbauer, D., Winkler, E., Novak, B., Steiner, H. and Haass, C. (2004) Embo. J., 23, 4738-48.

16. Niimura, M., Isoo, N., Takasugi, N., Tsuruoka, M., Ui-Tei, K., Saigo, K., Morohashi, Y ., Tomita, T. and Iwatsubo, T. (2005) J. Biol. Chem.

17. Crystal, A. S., Morais, V. A., Fortna, R. R., Carlin, D., Pierson, T. C., Wilson, C. A., Lee, V. M. and Doms, R. W. (2004) Biochemistry 43, 3555-63.

18. Lai, C. and Feng, L. (2004) Biochem. Biophys. Res. Commun., 314, 535-42.

19. Song, W ., Nadeau, P., Yuan, M., Yang, X., Shen, J. and Yankner, B. A. (1999) Proc. Natl. Acad. Sci. USA., 96, 6959-63.

20. Kopan, R. and lagan, M. X. (2004) Nat. Rev. Mol. Cell. Biol., 5, 499-504.

21. Kimberly, W. T. and Wolfe, M. S. (2003) J. Neurosci. Res., 74, 353-60.

22. Haass, C. (2004) Embo. J., 23, 483-8.

23. Selkoe, D. J. (2001) Proc. Natl. Acad. Sci. USA., 98,11039-41.

24. Thinakaran, G., Harris, C. L., Ratovitski, T., Davenport, F., Slunt, H. H., Price, D. L., Borchelt, D. R. and Sisodia, S. S. (1997) J. Biol. Chem., 272, 28415-22.

25. Ratovitski, T., Slunt, H. H., Thinakaran, G., Price, D. L., Sisodia, S. S. and Borchelt, D. R. (1997) J. Biol. Chem. 272, 24536-41.

26. Thinakaran, G., Borchelt, D. R., Lee, M. K., Slunt, H. H., Spitzer, L., Kim, G., Ratovitsky, T., Davenport, F., Nordstedt, C., Seeger, M., Hardy, J., Levey, A. I., Gandy, S. E., Jenkins, N. A., Copeland, N. G., Price, D. L. and Sisodia, S. S. (1996) Neuron 17, 181-90.

27. Kim, T. W., Pettingell, W. H., Hallmark, 0. G., Moir, R. D., Wasco, W. and Tanzi, R. E. (1997) J. Biol. Chem., 272, 11006-10.

28. Nyabi, O., Bentahir, M., Horre, K., Herreman, A., Gottardi-Littell, N., Van Broeckhoven, C., Merchiers, P., Spittaels, K., Annaert, W. and De Strooper, B. (2003) J. Biol. Chem., 278, 43430-6.

29. Honda, T ., Yasutake, K., Nihonmatsu, N., Mercken, M., Takahashi, H., Murayama, O., Murayama, M., Sato, K., Omori, A., Tsubuki, S., Saido, T. C. and Takashima, A. (1999) J. Neurochem., 72, 255-61.

30. Steiner, H., Romig, H., Pesold, B., Philipp, U., Baader, M., Citron, M., Loetscher, H., Jacobsen, H. and Haass, C.'(1999) Biochemistry 38, 14600-5.

31. Podlisny, M. B., Citron, M., Amarante, P., Sherrington, R., Xia, W., Zhang, J., Diehl, T., Levesque, G., Fraser, P., Haass, C., Koo, E. H., Seubert, P., St George-Hyslop, P., Teplow, D. B. and Selkoe, D. J. (1997) Neurobiol. Dis., 3, 325-37.

32. Campbell, W. A., Iskandar, M. K., Reed, M. L. and Xia, W. (2002) Biochemistry 41, 3372-9.

33. Liu, C. W., Corboy, M. J., DeMartino, G. N. and Thomas, P. J. (2003) Science 299, 408-11.

34. Saura, C. A., Tomita, T., Davenport, F., Harris, C. L., Iwatsubo, T. and Thinakaran, G.(1999) J. Biol. Chem., 274, 13818-23.

35. Steiner, H., Capell, A., Pesold, B., Citron, M., Kloetzel, P. M., Selkoe, D. J., Romig, H., Mendla, K. and Haass, C. (1998) J. Biol. Chem., 273, 32322-31.

36. Capell, A., Grunberg, J., Pesold, B., Diehlmann, A., Citron, M., Nixon, R., Beyreuther, K., Selkoe, D. J. and Haass, C. (1998) J. Biol. Chem., 273, 3205-11.

37. Kim, S. H., Lah, 1. J., Thinakaran, G., Levey, A. and Sisodia, S. S. (2000) Neurobiol. Dis., 7, 99-117.

38. Kirschenbaum, F., Hsu, S. C., Cordell, B. and McCarthy, J. V. (2001) J. Biol. Chem., 276, 7366-75.

39. Walter, J., Grunberg, J., Capell, A., Pesold, B., Schindzielorz, A., Citron, M., Mendla, K., George-Hyslop, P. S., Multhaup, G., Selkoe, D. J. and Haass, C. (1997) Proc. Natl. Acad. Sci. USA., 94, 5349-54.

40. Loetscher, H., Deuschle, U., Brockhaus, M., Reinhardt, D., Nelboeck, P., Mous, J., Grunberg, J., Haass, C. and Jacobsen, H. (1997) J. Biol. Chem. 272, 20655-9.

41. Hartmann, H., Busciglio, J., Baumann, K. H., Staufenbiel, M. and Yankner, B. A. (1997) J. Biol. Chem., 272, 14505-8.

42. Nornes, S., Groth, C., Camp, E., Ey, P. and lardelli, M. (2003) Exp. Cell. Res. 289, 124-32.

43. Mah, A. L., Perry, G., Smith, M. A. and Monteiro, M. J. (2000) J. Cell Biol. 151, 847-62.

44. Bertram, L., Hiltunen, M., Parkinson, M., Ingelsson, M., Lange, C., Ramasamy, I S., Mullin, I S., Menon, R., Sampson, A. J., Hsiao, M. Y ., Elliott, I S. J., Velicelebi, G., Moscarillo, T., Hyman, B. T., Wagner, S. L., Becker, K. D., Blacker, D. and Tanzi, R. E. (2005) N. Engl. J. Med., 352, 884-94.

45. Kleijnen, M. F., Shih, A. H., Zhou, P., Kumar, S., Soccio, R. E., Kedersha, N. L., Gill, G. and Howley, P. M. (2000) Mol. Cell, 6,,409-19.

46. Walters, K. J., Kleijnen, M. F., Goh, A. M., Wagner, G. and Howley, P. M. (2002) Biochemistry, 41,1767-77.

47. Kleijnen, M. F., Alarcon, R. M. and Howley, P. M. (2003) Mol. Biol. Cell, 14, 3868-75.

48. Massey, L. K., Mah, A. L., Ford, D. L., Miller, J., Doong, H., and Monteiro, M. J. (2004) Journal of Alzheimer's Disease 6, 79-92.

49. Janicki, S. and Monteiro, M. J. (1997) J. Cell Biol., 139, 485-95.

50. Shirotani, K., Edbauer, D., Capell, A., Schmitz, J., Steiner, H. and Haass, C. (2003) J. Biol. Chem., 278, 16474-7.

References Cited in Disclosure Relating to Ubiquilin Suppression of Polyglutamine-Induced Toxicity

1a. Bates, G., Harper, P. & Jones, L. Huntington's Disease, (Oxford University Press, Oxford, 2002).

2a. A novel gene containing a trinucleotide repeat that is expanded and unstable on Huntington's disease chromosomes. The Huntington's Disease Collaborative Research Group. Cell 72, 971-83 (1993).

3a. Ikeda, H. et al. Expanded polyglutamine in the Machado-Joseph disease protein induces cell death in vitro and in vivo. Nat Genet 13, 196-202 (1996).

4a. Ordway, J. M., Cearley, J. A. & Detloff, P. J. CAG-polyglutamine-repeat mutations: independence from gene context. Philos Trans R Soc Lond B Biol Sci 354, 1083-8 (1999).

5a. Landles, C. & Bates, G. P. Huntingtin and the molecular pathogenesis of Huntington's disease. Fourth in molecular medicine review series. EMBO Rep 5, 958-63 (2004).

6a. Michalik, A. & Van Broeckhoven, C. Pathogenesis of polyglutamine disorders: aggregation revisited. Hum Mol Genet 12 Spec No 2, R173-86 (2003).

7a. La Spada, A. R. & Taylor, J. P. Polyglutamines placed into context. Neuron 38, 681-4 (2003).

8a. Ross, C. A. Polyglutaminie pathogenesis: emergence of unifying mechanisms for Huntington's disease and related disorders. Neuron 35, 819-22 (2002).

9a. Ross, C. A. & Poirier, M. A. Protein aggregation and neurodegenerative disease. Nat Med 10 Suppl, S10-7 (2004).

10a. Arrasate, M., Mitra, S., Schweitzer, E. S., Segal, M. R. & Finkbeiner, S. Inclusion body formation reduces levels of mutant huntingtin and the risk of neuronal death. Nature 431, 805-10 (2004).

11a. Bowman, A. B., Yoo, S. Y., Dantuma, N. P. & Zoghbi, H. Y. Neuronal dysfunction in a polyglutamine disease model occurs in the absence of ubiquitin-proteasome system impairment and inversely correlates with the degree of nuclear inclusion formation. Hum Mol Genet 14, 679-91 (2005).

12a. Wexler, N. S. et al. Venezuelan kindreds reveal that genetic and environmental factors modulate Huntington's disease age of onset. Proc Natl Acad Sci USA 101, 3498-503 (2004).

13a. Davidson, J. D. et al. Identification and characterization of an ataxin-1-interacting protein: A1Up, a ubiquitin-like nuclear protein. Hum Mol Genet 9, 2305-12 (2000).

14a. Doi, H. et al. Identification of ubiquitin-interacting proteins in purified polyglutamine aggregates. FEBS Lett 571, 171-6 (2004).

15a. Mangiarini, L. et al. Exon 1 of the HD gene with an expanded CAG repeat is sufficient to cause a progressive neurological phenotype in transgenic mice. Cell 87, 493-506 (1996).

16a. Hazeki, N., Nakamura, K., Goto, J. & Kanazawa, I. Rapid aggregate formation of the huntingtin N-terminal fragment carrying an expanded polyglutamine tract. Biochem Biophys Res Commumn 256, 361-6 (1999).

17a. Atabay, C., Cagnoli, C. M., Kharlamov, E., Ikonomovic, M. D. & Manev, H. Removal of serum from primary cultures of cerebellar granule neurons induces oxidative stress and DNA fragmentation: protection with antioxidants and glutamate receptor antagonists. J Neurosci Res 43, 465-75 (1996).

18a. lacovitti, L., Stull, N. D. & Johnston, K. Melatonin rescues dopamine neurons from cell death in tissue culture models of oxidative stress. Brain Res 768, 317-26 (1997).

19a. Andoh, T., Chock, P. B. & Chiueh, C. C. The roles of thioredoxin in protection against oxidative stress-induced apoptosis in SH-SY5Y cells. J Biol Chem 277, 9655-60 (2002).

20a. Pandey, S., Lopez, C. & Jammu, A. Oxidative stress and activation of proteasome protease during serum deprivation-induced apoptosis in rat hepatoma cells; inhibition of cell death by melatonin. Apoptosis 8, 497-508 (2003).

21a. Piret, J. P. et al. Hypoxia and CoCl2 protect HepG2 cells against serum deprivation- and t-BHP-induced apoptosis: a possible anti-apoptotic role for HIF-1. Exp Cell Res 295, 340-9 (2004).

22a. Giuliano, P. et al. DNA damage induced by polyglutamine-expanded proteins. Hum Mol Genet 12, 2301-9 (2003).

23a. Satyal, S. H. et al. Polyglutamine aggregates alter protein folding homeostasis in Caenorhabditis elegans. Proc Natl Acad Sci USA 97, 5750-5 (2000).

24a. Morley, J. F., Brignull, H. R., Weyers, J. J. & Morimoto, R. I. The threshold for polyglutamine-expansion protein aggregation and cellular toxicity is dynamic and influenced by aging in Caenorhabditis elegans. Proc Natl Acad Sci USA 99, 10417-22 (2002).

25a. Caldwell, G. A. et al. Suppression of polyglutamine-induced protein aggregation in Caenorhabditis elegans by torsin proteins. Hum Mol Genet 12, 307-19 (2003).

26a. Yamanaka, K., Okubo, Y., Suzaki, T. & Ogura, T. Analysis of the two p97/VCP/Cdc48p proteins of Caenorhabditis elegans and their suppression of polyglutamine-induced protein aggregation. J Struct Biol 146, 242-50 (2004).

27a. Okkema, P. G., Harrison, S. W., Plunger, V., Aryana, A. & Fire, A. Sequence requirements for myosin gene expression and regulation in Caenorhabditis elegans. Genetics 135, 385-404 (1993).

28a. Timmons, L. & Fire, A. Specific interference by ingested dsRNA. Nature 395, 854 (1998).

29a. Timmons, L., Court, D.L. & Fire, A. Ingestion of bacterially expressed dsRNAs can produce specific and potent genetic interference in Caenorhabditis elegans. Gene 263, 103-12 (2001).

30a. Bertram, L. et al. Family-based association between Alzheimer's disease and variants in UBQLN1. N Engl J Med 352, 884-94 (2005).

31a. Mah, A. L., Perry, G., Smith, M. A. & Monteiro, M. J. Identification of ubiquilin, a novel presenilin interactor that increases presenilin protein accumulation. J Cell Biol 151, 847-62 (2000).

32a. Hay, D. G. et al. Progressive decrease in chaperone protein levels in a mouse model of Huntington's disease and induction of stress proteins as a therapeutic approach. Hum Mol Genet 13, 1389-405 (2004).

33a. Gokhale, K. C., Newnam, G. P., Sherman, M. Y. & Chernoff, Y. O. Modulation of prion-dependent polyglutamine aggregation and toxicity by chaperone proteins in the yeast model. J Biol Chem 280, 22809-18 (2005).

34a. Thomas, M. et al. The unfolded protein response modulates toxicity of the expanded glutamine androgen receptor. J Biol Chem (2005).

35a. Sakahira, H., Breuer, P., Hayer-Hartl, M. K. & Hartl, F. U. Molecular chaperones as modulators of polyglutamine protein aggregation and toxicity. Proc Natl Acad Sci U S A 99 Suppl 4, 16412-8 (2002).

36a. Kleijnen, M. F., Alarcon, R. M. & Howley, P. M. The ubiquitin-associated domain of hPLIC-2 interacts with the proteasome. Mol Biol Cell 14, 3868-75 (2003).

37a. Chen, L. & Madura, K. Rad23 promotes the targeting of proteolytic substrates to the proteasome. Mol Cell Biol 22, 4902-13 (2002).

38a. Elsasser, S., Chandler-Militello, D., Muller, B., Hanna, J. & Finley, D. Rad23 and Rpn10 serve as alternative ubiquitin receptors for the proteasome. J Biol Chem 279, 26817-22 (2004).

39a. Verma, R., Oania, R., Graumann, J. & Deshaies, R. J. Multiubiquitin chain receptors define a layer of substrate selectivity in the ubiquitin-proteasome system. Cell 118, 99-110 (2004).

40a. Kaye, F. J. et al. A family of ubiquitin-like proteins binds the ATPase domain of Hsp70-like Stch. FEBS Lett 467, 348-55 (2000).

41a. Massey, L. K. et al. Overexpression of ubiquilin decreases ubiquitination and degradation of presenilin proteins. J Alzheimers Dis 6, 79-92 (2004).

42a. Ko, H. S., Uehara, T. & Nomura, Y. Role of ubiquilin associated with protein-disulfide isomerase in the endoplasmic reticulum in stress-induced apoptotic cell death. J Biol Chem 277, 35386-92 (2002).

43a. Massey, L. K., Mah, A. L. & Monteiro, M. J. Ubiquilin regulates presenilin endoproteolysis and modulates gamma-secretase components, Pen-2 and Nicastrin. Biochem J (2005).

44a. Slifer, M. A., Martin, E. R., Haines, J. L. & Pericak-Vance, M. A. The ubiquilin 1 gene and Alzheimer's disease. N Engl J Med 352, 2752-3; author reply 2752-3 (2005).

45a. Narain, Y., Wyttenbach, A., Rankin, J., Furlong, R. A. & Rubinsztein, D. C. A molecular investigation of true dominance in Huntington's disease. J Med Genet 36, 739-46 (1999).

46a. Mello, C. & Fire, A. DNA transformation. Methods in Cell Biology 48, 451-82 (1995).

1. A method of decreasing fragmentation of full presenilin 1 and/or 2 proteins; the method comprising
introducing an expression vector to a host cell that expresses presenilin 1 and/or 2, wherein the expression vector comprises a nucleotide sequence encoding ubiquilin;
maintaining the transformed host cell under biological conditions sufficient for expression and accumulation of the ubiquilin in the host cell; and
measuring the level of presenilin 1 and/2 fragments relative to a host cell not expressing increased levels of ubiquilin.

2. The method according to claim 1, wherein the expression vector comprises a nucleotide sequence that encodes polypeptides comprising the amino acid residue sequence of SEQ ID NOs: 2 or 4 (amino acid sequences of ubiquilin 595 and 589, respectively) or a fragment, or variant having at least 90% homology comprising a thereof that has the same functional activity of ubiquilin.

3. The method according to claim 1, wherein the nucleotide sequence comprises SEQ ID NO 1 or 3, or a sequence complementary to such sequences or hybridizes thereto under stringent hybridization conditions.

4. A method for increasing full length presenilin 1 and/or 2 proteins by reducing fragmentation of the full length presenilin 1 and/or 2 proteins, the method comprising;
introducing an expression vector to a host cell that expresses presenilin 1 and/or 2, wherein the expression vector comprises a nucleotide sequence encoding ubiquilin;
maintaining the transformed host cell under biological conditions sufficient for expression and accumulation of the ubiquilin in the host cell; and
measuring the level of full length presenilin 1 and/2 relative to a host cell not expressing increased levels of ubiquilin.

5. A method for decreasing levels of Pen-2 and/or Nicastrin, the method comprising increasing levels of ubiquilin in a cell that expresses Pen-2 and/or Nicastrin.

6. A method for reducing catalytic γ-secretase enzyme in a cell, the method comprising introducing a sufficient amount of ubiquilin to reduce the endoproteolysis formation of presenilin 1 and/or 2 fragments.

7. A host cell with increased levels of full length Presenilin 1 and/or 2 and increased levels of ubiquilin.

8. A method for decreasing cell death in a cell exhibiting aggregation of polyglutamine-containing proteins, the method comprising;
introducing an expression vector to a host cell comprising a nucleotide sequence encoding ubiquilin in an amount to overexpress ubiquilin; and
maintaining the transformed host cell under biological conditions sufficient for expression and accumulation of the ubiquilin in the host cell, wherein overexpresion of ubiquilin reduces sensitivity of cell to stress induced by expanded polyglutamine proteins.

9. The method of claim 8, wherein the expression vector comprises a nucleotide sequence that encodes polypeptides comprising the amino acid residue sequence of Ubiquilin, or variants having at least 90% homology and having the same functional activity of ubiquilin, or fragments thereof.

10. The method according to claim 8 wherein the nucleotide sequence is SEQ ID NOs: 5, 7, 9, 13, 14, or 17.

11. A method for determining the effectiveness of ubiquilin in reducing polyglutamine expansion in a host cell, the method comprising:
introducing an expression vector to a host cell comprising a nucleotide sequence encoding ubiquilin;
maintaining the transformed host cell under biological conditions sufficient for expression and accumulation of the ubiquilin in the host cell; and
measuring the level of cell death in the host cells relative to a host cell not expressing increased levels of ubiquilin.

12. Use of an expression vector encoding for a ubiquilin protein or variant thereof having deletions or substitution but maintaining the functionality of ubiquilin, in a medicament for the treatment of neurological disorder including HD.
